(19) United States
(12) Patent Application Publication
(10) Pub. No.: US 2020/0397852 A1
(43) Pub. Date: Dec. 24, 2020

(54) DOSING REGIMENS FOR CELIAC DISEASE

(71) Applicant: ImmusanT, Inc., Cambridge, MA (US)

(72) Inventor: Robert P. Anderson, Shrewsbury, MA (US)

(73) Assignee: ImmusanT, Inc., Cambridge, MA (US)

(21) Appl. No.: 16/862,783

(22) Filed: Apr. 30, 2020

Related U.S. Application Data
(63) Continuation of application No. PCT/US2018/058183, filed on Oct. 30, 2018.
(60) Provisional application No. 62/745,248, filed on Oct. 12, 2018.
(60) Provisional application No. 62/578,549, filed on Oct. 30, 2017.

(51) Int. Cl. A61K 38/10; A61K 9/00; A61P 1/00

(52) U.S. Cl. CPC A61K 38/10; A61P 1/00; A61K 9/0019

(57) ABSTRACT

Provided herein are compositions and methods for treating subjects with Celiac disease, e.g., specific dosages and dosage schedules of a composition comprising at least one gluten peptide for use in treating subjects with Celiac disease.

### RELATED APPLICATIONS

This application claims the benefit under 35 U.S.C. § 119(e) of U.S. provisional application No. 62/578,549, filed Oct. 30, 2017, and U.S. provisional application No. 62/745,248, filed Oct. 12, 2018, the contents of each of which are incorporated by reference herein in their entirety.

### BACKGROUND

Celiac disease, also known as coeliac disease or Celiac sprue (Coeliac sprue), affects approximately 1% of people in Europe and North America. In many of those affected, Celiac disease is unrecognised, but this clinical oversight is now being rectified with greater clinical awareness. A gluten free diet is the only currently approved treatment for Celiac disease, and because regular ingestion of as little as 50 mg of gluten (equivalent to 1/100^{th} of a standard slice of bread) can damage the small intestine; chronic inflammation of the small bowel is commonplace in subjects on a gluten free diet. Persistent inflammation of the small intestine has been shown to increase the risk of cancer, osteoporosis and death. As gluten is so widely used, for example, in commercial soups, sauces, ice-creams, etc., maintaining a gluten-free diet is difficult.

Celiac disease generally occurs in genetically susceptible individuals who possess either HLA-DQ2.5 (encoded by the genes HLA-DQA1*05 and HLA-DQB1*02) accounting for about 90% of individuals, HLA-DQ2.2 (encoded by the genes HLA-DQA1*02 and HLA-DQB1*02), or HLA-DQ8 (encoded by the genes HLA-DQA1*03 and HLA-DQB1*0302). Without wishing to be bound by theory, it is believed that such individuals mount an inappropriate HLA-DQ2- and/or DQ8-restricted CD4⁺ T cell-mediated immune response to peptides derived from aqueous-insoluble proteins of wheat flour, gluten, and related proteins in rye and barley.

### SUMMARY

Described herein are specific dosages and dosage schedules of a composition comprising at least one gluten peptide for use in treating subjects with Celiac disease. In some embodiments of any one of the methods provided, the composition comprises at least one peptide comprising at least one amino acid sequence selected from PFPQPELPY (SEQ ID NO: 4), PQPELPYPQ (SEQ ID NO: 5), PFPQPEQPF (SEQ ID NO: 6), PQPEQPFPW (SEQ ID NO: 7), PIPEQPQPY (SEQ ID NO: 8) and EQPIPEQPQ (SEQ ID NO: 9). In some embodiments of any one of the methods provided, the composition comprises at least one peptide selected from a first peptide comprising the amino acid sequence PFPQPELPY (SEQ ID NO: 4) and/or PQPELPYPQ (SEQ ID NO: 5); a second peptide comprising the amino acid sequence PFPQPEQPF (SEQ ID NO: 6) and/or PQPEQPFPW (SEQ ID NO: 7); and a third peptide comprising the amino acid sequence PIPEQPQPY (SEQ ID NO: 8) and/or EQPIPEQPQ (SEQ ID NO: 9). In some embodiments of any one of the methods provided, the composition comprises the first, second and third peptides. In some embodiments of any one of the methods provided, the composition comprises a first peptide comprising the amino acid sequence ELQPFPQPELPYPQPQ (SEQ ID NO: 1), wherein the N-terminal glutamate is a pyroglutamate and the carboxyl group of the C-terminal glutamine is amidated; a second peptide comprising the amino acid sequence EQPFPQPEQPFPWQP (SEQ ID NO: 2), wherein the N-terminal glutamate is a pyroglutamate and the carboxyl group of the C-terminal proline is amidated; and a third peptide comprising the amino acid sequence EPEQPIPEQPQPYPQQ (SEQ ID NO: 3), wherein the N-terminal glutamate is a pyroglutamate and the carboxyl group of the C-terminal glutamine is amidated.

Without being bound by theory, it is believed that administration of the compositions provided herein according to the dosages and dosage schedules described herein to a subject with Celiac disease can induce immune tolerance in the subject such that the subject may consume or come into contact with wheat, rye, and/or barley and, optionally, oats without a significant T cell response which would normally lead to symptoms of Celiac disease. In particular, in addition to a tolerizing dose period of the composition, a dose escalation period is contemplated prior to the tolerizing dose to gradually increase the dose administered to the subject (e.g., to reduce side effects).

Accordingly, aspects of the disclosure relate to compositions and methods for treating a subject with Celiac disease. In some aspects, any one of the methods provided herein is a method for treating Celiac disease in a subject.

In some embodiments of any one of the methods provided, the method comprises administering to a subject, such as one having a homozygous HLA-DQ2.5 genotype or a non-homozygous HLA-DQ2.5 genotype. In some embodiments of any one of the methods provided, the subject is HLA-DQ2.5 positive. In some embodiments of any one of the methods provided, the non-homozygous HLA-DQ2.5 genotype is a heterozygous HLA-DQ2.5 genotype. In some embodiments of any one of the methods provided, the heterozygous HLA-DQ2.5 genotype is HLA-DQ2.5/2.2, HLA-DQ2.5/7, or HLA-DQ2.5/8.

In some embodiments of any one of the methods provided, the subject is on a gluten-free diet.

In some embodiments of any one of the methods provided, the second composition is administered at least six, seven, eight, nine or ten times to the subject.

In some embodiments of any one of the methods provided, the time between doses of a gluten peptide composition to the subject is at least 1, 2, 3, 4 or 5 days.

In some embodiments of any one of the methods provided,

(i) the first peptide comprises the amino acid sequence ELQPFPQPELPYPQPQ (SEQ ID NO: 1), wherein the N-terminal glutamate is a pyroglutamate and the C-terminal glutamine is amidated;

(ii) the second peptide comprises the amino acid sequence EQPFPQPEQPFPWQP (SEQ ID NO: 2), wherein the N-terminal glutamate is a pyroglutamate and the C-terminal proline is amidated; and

(iii) the third peptide comprises the amino acid sequence EPEQPIPEQPQPYPQQ (SEQ ID NO: 3), wherein the N-terminal glutamate is a pyroglutamate and the C-terminal glutamine is amidated.

In some embodiments of any one of the methods provided herein, each composition comprising one or more gluten peptides can comprise or consist of the aforementioned first, second, and third peptides. In some embodiments of any one of the methods provided, the first, second and third peptides are in equimolar amounts in each of compositions comprising one or more gluten peptides.

In some embodiments of any one of the methods provided, each of the compositions comprising one or more gluten peptides are/is administered intradermally. In some embodiments of any one of the methods provided, the compositions comprising one or more gluten peptides are/is administered as a bolus by intradermal injection. In some embodiments of any one of the methods provided, each of the compositions comprising one or more gluten peptides are/is formulated as a sterile, injectable solution. In some embodiments of any one of the methods provided, the sterile, injectable solution is sodium chloride. In some embodiments of any one of the methods provided, the sodium chloride is sterile sodium chloride 0.9% USP.

In some embodiments of any one of the methods provided, the second composition is administered for at least 3, 4, 5 or 6 weeks. In some embodiments of any one of the methods provided, the time between doses of the second composition to the subject is at least 1, 2, 3, 4 or 5 days. In some embodiments of any one of the methods provided, the second composition is administered at least once, twice or three times a week for at least 3, 4, 5 or 6 weeks.

In some embodiments of any one of the methods provided, the method further comprises administering a composition comprising wheat, barley and/or rye (e.g., a composition comprising 6 grams of gluten) to the subject after the second composition is administered. In some embodiments of any one of the methods provided, the administration of the composition comprising wheat, barley and/or rye is for at least 4, 5, 6, 7 or 8 weeks.

Also provided herein in an aspect is a method of treating a subject with Celiac disease, the method comprising any one of the titration or dose escalation phases provided herein, comprising any one of the tolerizing phases provided herein, or both any one of the titration phases and any one of the tolerizing phases provided herein.

In an embodiment of any one of the methods provided herein, the gluten peptide composition may be any one of the gluten peptide compositions provided herein. This embodiment includes the methods of the claims where an alternative gluten peptide compositions may substitute the gluten peptide composition recited, such alternative gluten peptide compositions may be any one of the gluten peptide compositions provided herein.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is an exemplary schematic of a study design to evaluate dose titration and push dose.

FIG. 2 is an exemplary schematic of a study design to evaluate dose titration.

FIG. 3 is a graph showing dosage numbers and dosage amounts (micrograms) in dosage administration studies. Incorporation of an up-dosing regimen enabled patients to achieve and maintain 6 times higher dose versus a fixed-dose regimen.

FIG. 4 is a series of graphs depicting plasma concentrations of gluten peptide compositions before and after dosing.

FIG. 5 is a graph depicting incidence and severity of adverse events in subjects receiving an up-dosing regimen of gluten peptide composition.

FIG. 6 is a graph depicting IL-2 level in subjects receiving an up-dosing regimen of gluten peptide composition.

FIG. 7 is a series of graphs depicting IL-2 release in plasma in fixed dosing (left and middle panels) and up-dosing (right panel) regimens.

FIG. 8 is a graph depicting Gastrointestinal Symptom Rating Scale (GSRS) score over time (lower numbers indicate lesser symptom severity). Overall symptom scores were measured at baseline and then weekly. Placebo patients pooled all cohorts. Updosing begins at 3 micrograms and the top dose was 900 micrograms. A significant reduction in symptoms compared to baseline was seen. No difference in symptoms between baseline and treatment period was seen in the placebo group.

FIG. 9 is a table summarizing the weekly GI symptom diary across treatment period related to pain or discomfort.

FIG. 10 is a table summarizing the weekly GT symptom diary across treatment period related to nausea.

FIG. 11 is a graph depicting no difference in duodenal morphometry in Cohort 3 (n=10 CeD patients).

FIG. 12 shows a study schematic. *Escalation was amended for all cohorts by including 3 μg and 9 μg doses when one participant in Cohort 1 withdrew with gastrointestinal adverse events graded moderate or severe after 30 μg and 60 μg doses. V14 was 1 week after V12. EOS, end of study; EOT, end of treatment; V, visit.

FIG. 13 is a series of graphs showing incidence, severity, and organ class of treatment-emergent adverse events after each dose. Treatment-emergent adverse events after each dose of Nexvax2 or placebo are shown as the number of participants who experienced no, mild, moderate, severe, or serious treatment-emergent adverse events in (A), (C), (E), (G), (I), and (K) and as the total number of treatment-emergent adverse events classified by organ system in (B), (D), (F), (H), (J), and (L). PT, post-treatment.

FIG. 14 is a heat map showing the median fold change in plasma cytokines and chemokines following administration of Nexvax2. Assessments were made during the escalation phase, at 150 μg of Nexvax2 (previously defined maximum tolerated dose), and after the first, second, forth, and eighth administrations at the 300 μs and 900 μg maintenance doses. Plasma cytokines and chemokines were measured pre-treatment, and at 4, 6, and 10 hours post-treatment.

FIG. 15 is a series of graphs showing plasma concentrations of Nexvax2 peptides. Plasma concentrations of NPL001, NPL002, and NPL003 peptides at 45 minutes after intradermal administration of Nexvax2 in cohort 3 (n=10). Mean (95% CI) concentrations are shown for NPL001 (A), NPL002 (B), and NPL003 (C) after escalating doses of Nexvax2, and at the maintenance dose of 900 μs. The LLOQ for each peptide was 2 ng/mL; readings below the LLOQ were assigned 2 ng/mL. Pre-treatment plasma concentrations of Nexvax2 peptides were below the LLOQ for each of the indicated doses in all participants. LLOQ, lower limit of quantitation.

FIG. 16: is a diagram showing a trial profile. For cohort 1 and cohort 2, the Nexvax2 starting dose was 30 μg; for cohort 1′ and cohort 2′, the Nexvax2 starting dose was 3 μg.

FIG. 17 is a diagram showing the schedule of assessments. The schedule of assessments for screening, treatment, and follow-up periods were as follows: vital signs included pulse, blood pressure, respiratory rate, oxygen saturation, and temperature; 12-lead electrocardiogram; coeliac disease-specific serology included IgA specific for transglutaminase 2 and IgG specific for deamidated gliadin peptide; HLA-DQA and HLA-DQB genotyping; Coeliac Dietary Adherence Test; safety laboratory tests included hematology, blood urea, creatinine and electrolytes, albumin, total protein, alkaline phosphatase, aspartate aminotransferase, alanine aminotransferase, total bilirubin, direct bilirubin, prothrombin time and partial thromboplastin time, and at visit 1, glucose, calcium, cholesterol, triglycerides, phosphorus, lactate dehydrogenase, uric acid, and thyroid-stimulating hormone; urinalysis by dipstick; urinary pregnancy test (β-hCG) for females; Gastrointestinal Symptom Rating Scale score; cytokine and chemokine 38plex; immune cell counting in blood; anti-Nexvax2 IgG and IgA; and plasma pharmacokinetics of NPL001, NPL002, and NPL003 at pre-treatment and 45 minutes post-treatment. ADA, anti-Nexvax2 IgG and IgA; CDAT, Coeliac Dietary Adherence Test; CK, cytokine and chemokine 38plex; CS, coeliac disease-specific serology; ECG, electrocardiogram; GSRS, Gastrointestinal Symptom Rating Scale; IC, immune cell counting; PK, pharmacokinetics; Preg, urinary pregnancy test; S'lab, safety laboratory tests; V, visit; VS, vital signs. *Indicates visits when VS and CK, and where indicated, S'lab and IC were assessed pre-treatment and 4 hours post-treatment. **Indicates visits when VS, CK, IC, and S'lab were assessed pre-treatment and post-treatment at 4, 6, and 10 hours.

FIG. 18: is a series of graphs showing weekly Gastrointestinal Symptom Rating Scale (GSRS) scores. Average GSRS scores are shown as median and interquartile range for participants who received placebo or Nexvax2 with a starting dose of 3 μg. The GSRS is a self-assessed rating of 15 digestive symptoms over the previous week, where 1 represents the most positive option and 7 the most negative. The GSRS was completed on the first day of the screening period (screen), at baseline on the first day of the treatment period before dosing (BSL), and weekly before dosing during the treatment period. GSRS data up to the 6th week of the treatment period were combined for the nine placebo-treated participants.

FIG. 19 is a heatmap showing fold change in plasma cytokines and chemokines following administration of the first and last maintenance doses of Nexvax2. Fold change from pre-treatment levels to four hours post-treatment in plasma concentrations of 38 cytokines and chemokines in individual participants after administration of Nexvax2 or placebo.

FIG. 20 is a series of graphs showing Nexvax2-specific IgG and IgA. In cohort 3 (n=10), serial serum anti-Nexvax2 IgG (A) and IgA (B) over the 60-day treatment period did not change significantly with Nexvax2 treatment. The cutoff set as the 95% CI in sera from healthy donors is indicated. Day 36 was the first 900 μg maintenance dose of Nexvax2; day 60 was the eighth 900 μg maintenance dose of Nexvax2.

FIG. 21 is a series of graphs showing the relationship between plasma concentrations of Nexvax2 peptides. Plasma concentrations of NPL001, NPL002, and NPL003 peptides at 45 minutes after intradermal administration of Nexvax2 in cohort 3 (n=10). The relationships between concentrations of NPL001, NPL002, and NPL003 measured in the same plasma samples are shown in (A-C). Concentrations of NPL001, NPL002, and NPL003 after the first (day 36) and eighth (day 60) 900 μg maintenance doses are shown in (D-F). The LLOQ for each peptide was 2 ng/mL; readings below the LLOQ were assigned 2 ng/mL. Pre-treatment plasma concentrations of Nexvax2 peptides were below the LLOQ for each of the indicated doses in all participants. LLOQ, lower limit of quantitation.

FIG. 22 is a series of graphs showing the relationship between Nexvax2-specific antibodies and Nexvax2 peptides. In cohort 3 (n=10), anti-Nexvax2 IgG and IgA were not significantly correlated with plasma concentrations of NPL001, NPL002, or NPL003 peptides 45 minutes after the first (day 36, closed symbols) and eighth (day 60, open symbols) 900 μg maintenance doses of Nexvax2. For all participants receiving Nexvax2 in cohort 3, serum anti-Nexvax2 IgG and IgA levels were below the cutoff set as the 95% CI in sera from healthy donors.

FIG. 23 shows the schematic of a study design containing HLA-DQ2.5 homozygous and non-homozygous arms.

FIG. 24 shows the schematic of a study design for comparison of subcutaneous and intradermal injection.

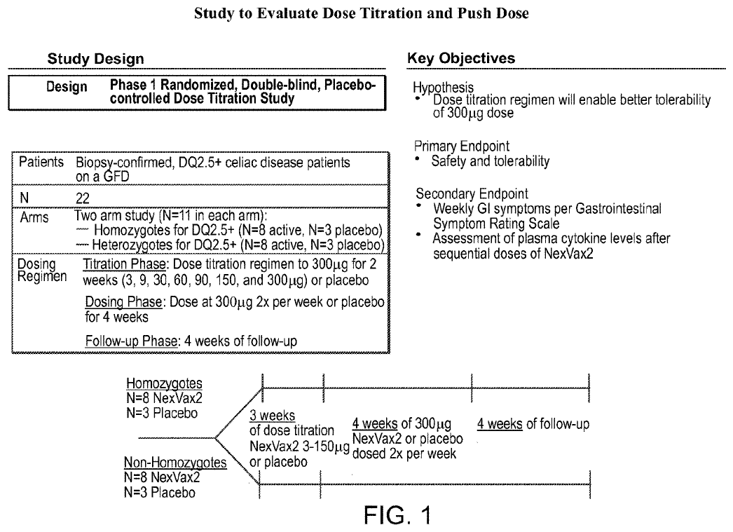

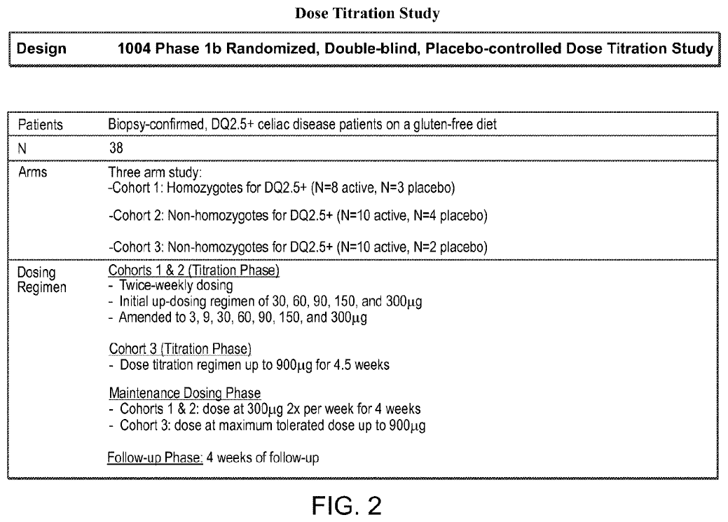

### DETAILED DESCRIPTION

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Unless otherwise indicated, techniques utilized in the present disclosure are standard procedures, well known to those skilled in the art. Such techniques are described and explained throughout the literature in sources such as, J. Sambrook et al., Molecular Cloning: A Laboratory Manual, Cold Spring Harbour Laboratory Press (2012); T. A. Brown (editor), Essential Molecular Biology: A Practical Approach, Volumes 1 and 2, IRL Press (2000 and 2002); D. M. Glover and B. D. Hames (editors), Current Protocols in Molecular Biology, Greene Pub. Associates and Wiley-Interscience (1988, including all updates until present); Edward A. Greenfield (editor) Antibodies: A Laboratory Manual. Cold Spring Harbour Laboratory, (2013); and J. E. Coligan et al. (editors), Current Protocols in Immunology, John Wiley & Sons (including all updates until present).

The term “Celiac disease” generally refers to an immune-mediated systemic disorder elicited by gluten and related prolamines in genetically susceptible individuals, characterized by the presence of a variable combination of gluten-dependent clinical manifestations, celiac disease-specific antibodies, human leukocyte antigen (HLA)-DQ2 and HLA-DQ8 haplotypes, and enteropathy. The disease encompasses a spectrum of conditions characterised by an inappropriate CD4⁺ T cell response to gluten, or a peptide thereof. The severe form of celiac disease is characterised by a flat small intestinal mucosa (hyperplastic villous atrophy) and other forms are characterised by milder histological abnormalities in the small intestine, such as intra-epithelial lymphocytosis without villous atrophy. Serological abnormalities associated with celiac disease generally include the presence of autoantibodies specific for tissue transglutaminase-2 and antibodies specific for deamidated gluten-derived peptides. The clinical manifestations associated with celiac disease can include fatigue, chronic diarrhoea, malabsorption of nutrients, weight loss, abdominal distension, anaemia as well as a substantially enhanced risk for the development of osteoporosis and intestinal malignancies (lymphoma and carcinoma).

The terms “human leukocyte antigen” and “HLA” are here defined as a genetic fingerprint expressed on human white blood cells, composed of proteins that play a critical role in activating the body's immune system to respond to foreign organisms. In humans and other animals, the HLA is also collectively referred to as the “major histocompatibility complex” (MHC).

The term “subject” includes inter alia an individual, patient, target, host or recipient regardless of whether the subject is a human or non-human animal including mammalian species and also avian species. The term “subject”, therefore, includes a human, non-human primate (for example, gorilla, marmoset, African Green Monkey), livestock animal (for example, sheep, cow, pig, horse, donkey, goat), laboratory test animal (for example, rat, mouse, rabbit, guinea pig, hamster), companion animal (for example, dog, cat), captive wild animal (for example, fox, deer, game animals) and avian species including poultry birds (for example, chickens, ducks, geese, turkeys). The preferred subject, however, is a human. In some embodiments, the subject is a human on a gluten-free diet. In some embodiments, the subject is a human who is HLA-DQ2.5 positive. In some embodiments, the subject is a human who is HLA-DQ2.5 positive and HLA-DQ8 negative. In some embodiments, the subject is a human who is HLA-DQ2.5 positive and HLA-DQ8 positive.

### Peptides

The terms “peptide”, “polypeptide”, and “protein” can generally be used interchangeably and also encompass pharmaceutical salts thereof. In some embodiments of any one of the methods or compositions provided herein, the term “peptide” is used to refer to relatively short molecules comprising less than 50, more preferably less than 25, amino acids.

The overall length of each peptide defined herein may be, for example, 7 to 50 amino acids, such as 7, 8, 9 10, 11, 12, 13, 14, 15, 16, 17, 18, 19, 20, 21, 22, 23, 24, 25, 30, 35, 40, 45, or 50 amino acids, or any integer in between. It is contemplated that shorter peptides may prove useful, particularly those that are 20 or fewer amino acids in length, in therapeutics to reduce the likelihood of anaphylaxis but longer peptides with multiple epitopes are likely to be as effective as multiple short peptides, for example, in functional T cell-based diagnostics in vitro.

It is believed that the peptides of the disclosure, such as those that comprise SEQ ID NOs: 1, 2, and 3, are capable of generating a T cell response in a subject having Celiac disease. Without wishing to be bound by theory, T cell responses in a subject with Celiac disease can be caused by T-cell receptor ligation of the minimal T cell epitopes present in SEQ ID NOs: 1, 2, and 3 that are presented by HLA-DQ2.5 on the surface of antigen presenting cells.

In some embodiments, a peptide is modified during or after translation or synthesis (for example, by farnesylation, prenylation, myristoylation, glycosylation, palmitoylation, acetylation, phosphorylation [such as phosphotyrosine, phosphoserine or phosphothreonine], amidation, derivatisalion by known protecting/blocking groups, proteolytic cleavage, linkage to an antibody molecule or other cellular ligand, and the like). Any of the numerous chemical modification methods known within the art may be utilised including, but not limited to, specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

The phrases “protecting group” and “blocking group” as used herein, refers to modifications to the peptide, which protect it from undesirable chemical reactions, particularly in vivo. Examples of such protecting groups include esters of carboxylic acids and boronic acids, ethers of alcohols and acetals, and ketals of aldehydes and ketones. Examples of suitable groups include acyl protecting groups such as, for example, furoyl, formyl, adipyl, azelayl, suberyl, dansyl, acetyl, theyl, benzoyl, trifluoroacetyl, succinyl and methoxysuccinyl; aromatic urethane protecting groups such as, for example, benzyloxycarbonyl (Cbz); aliphatic urethane protecting groups such as, for example, t-hutoxycarhonyl (Boc) or 9-fluorenylmethoxy-carbonyl (FMOC); pyroglutamate and amidation. Many other modifications providing increased potency, prolonged activity, ease of purification, and/or increased half-life will be known to the person skilled in the art.

The peptides may comprise one or more modifications, which may be natural post-translation modifications or artificial modifications. The modification may provide a chemical moiety (typically by substitution of a hydrogen, for example, of a C—H bond), such as an amino, acetyl, acyl, amide, carboxy, hydroxy or halogen (for example, fluorine) group, or a carbohydrate group. Typically, the modification is present on the N- and/or C-terminus. Furthermore, one or more of the peptides may be PEGylated, where the PEG (polyethyleneoxy group) provides for enhanced lifetime in the blood stream. One or more of the peptides may also be combined as a fusion or chimeric protein with other proteins, or with specific binding agents that allow targeting to specific moieties on a target cell. The peptide may also be chemically modified at the level of amino acid side chains, of amino acid chirality, and/or of the peptide backbone.

Particular changes can be made to the peptides to improve resistance to degradation or optimize solubility properties or otherwise improve bioavailability compared to the parent peptide, thereby providing peptides having similar or improved therapeutic, diagnostic and/or pharmacokinetic properties. A preferred such modification includes the use of an N-terminal pyroglutamate and/or a C-terminal amide (such as the respective N-terminal pyroglutamate and C-terminal glutamine of SEQ ID NOs: 1, 2, and 3). Such modifications have been shown previously to significantly increase the half-life and bioavailability of the peptides compared to the parent peptides having a free N- and C-terminus.

In a particular embodiment, a composition comprising a first peptide comprising the amino acid sequence ELQPFPQPELPYPQPQ (SEQ ID NO: 1), wherein the N-terminal glutamate is a pyroglutamate and the C-terminal glutamine is amidated (i.e., the free C-terminal COO is amidated); a second peptide comprising the amino acid sequence EQPFPQPEQPFPWQP (SEQ ID NO: 2), wherein the N-terminal glutamate is a pyroglutamate and the C-terminal proline is amidated (i.e., the free C-terminal COO is amidated); and a third peptide comprising the amino acid sequence EPEQPIPEQPQPYPQQ (SEQ ID NO: 3), wherein the N-terminal glutamate is a pyroglutamate and the C-terminal glutamine is amidated (i.e., the free C-terminal COO is amidated) is contemplated. In some embodiments, the first, second and/or third peptides consist essentially of or consist of the amino acid sequence of SEQ ID NO: 1, 2, or 3, respectively. Compositions are further described herein.

Certain peptides described herein may exist in particular geometric or stereoisomeric forms. The present disclosure contemplates all such forms, including cis-(Z) and trans-(E) isomers, R- and S-enantiomers, diastereomers, (D)-isomers, (L)-isomers, the racemic mixtures thereof, and other mixtures thereof, as, falling within the scope of the disclosure. Additional asymmetric carbon atoms may be present in a substituent, such as an alkyl group. All such isomers, as well as mixtures thereof, are intended to be included in this disclosure.

In another example, to prevent cleavage by peptidases, any one or more of the peptides may include a non-cleavable peptide bond in place of a particularly sensitive peptide bond to provide a more stable peptide. Such non cleavable peptide bonds may include beta amino acids.

In certain embodiments, any one or more of the peptides may include a functional group, for example, in place of the scissile peptide bond, which facilitates inhibition of a serine-, cysteine- or aspartate-type protease, as appropriate. For example, the disclosure includes a peptidyl diketone or a peptidyl keto ester, a peptide haloalkylketone, a peptide sulfonyl fluoride, a peptidyl boronate, a peptide epoxide, a peptidyl diazomethane, a peptidyl phosphonate, isocoumarins, benzoxazin-4-ones, carbamates, isocyantes, isatoic anhydrides or the like. Such functional groups have been provided in other peptide molecules, and general routes for their synthesis are known.

The peptides may be in a salt form, preferably, a pharmaceutically acceptable salt form. “A pharmaceutically acceptable salt form” includes the conventional non-toxic salts or quaternary ammonium salts of a peptide, for example, from non-toxic organic or inorganic acids. Conventional non-toxic salts include, for example, those derived from inorganic acids such as hydrochloride, hydrobromic, sulphuric, sulfonic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

### Peptide Production

The peptides can be prepared in any suitable manner. For example, the peptides can be recombinantly and/or synthetically produced.

The peptides may be synthesised by standard chemistry techniques, including synthesis by an automated procedure using a commercially available peptide synthesiser. In general, peptides may be prepared by solid-phase peptide synthesis methodologies which may involve coupling each protected amino acid residue to a resin support, preferably a 4-methylbenzhydrylamine resin, by activation with dicyclohexylcarbodiimide to yield a peptide with a C-terminal amide. Alternatively, a chloromethyl resin (Merrifield resin) may be used to yield a peptide with a free carboxylic acid at the C-terminal. After the last residue has been attached, the protected peptide-resin is treated with hydrogen fluoride to cleave the peptide from the resin, as well as deprotect the side chain functional groups. Crude product can be further purified by gel filtration, high pressure liquid chromatography (HPLC), partition chromatography, or ion-exchange chromatography.

If desired, and as outlined above, various groups may be introduced into the peptide of the composition during synthesis or during expression, which allow for linking to other molecules or to a surface. For example, cysteines can be used to make thioethers, histidines for linking to a metal ion complex, carboxyl groups for forming amides or esters, amino groups for forming amides, and the like.

The peptides may also be produced using cell-free translation systems. Standard translation systems, such as reticulocyte lysates and wheat germ extracts, use RNA as a template; whereas “coupled” and “linked” systems start with DNA templates, which are transcribed into RNA then translated.

Alternatively, the peptides may be produced by transfecting host cells with expression vectors that comprise a polynucleotide(s) that encodes one or more peptides. For recombinant production, a recombinant construct comprising a sequence which encodes one or more of the peptides is introduced into host cells by conventional methods such as calcium phosphate transfection, DEAE-dextran mediated transfection, microinjection, cationic lipid-mediated transfection, electroporation, transduction, scrape lading, ballistic introduction or infection.

One or more of the peptides may be expressed in suitable host cells, such as, for example, mammalian cells (for example, COS, CHO, BHK, 293 HEK, VERO, HeLa, HepG2, MDCK, W138, or NIH 3T3 cells), yeast (for example, Saccharomyces or Pichia), bacteria (for example, E. coli, P. pastoris, or B. subtilis), insect cells (for example, baculovirus in Sf9 cells) or other cells under the control of appropriate promoters using conventional techniques. Following transformation of the suitable host strain and growth of the host strain to an appropriate cell density, the cells are harvested by centrifugation, disrupted by physical or chemical means, and the resulting crude extract retained for further purification of the peptide or variant thereof.

Suitable expression vectors include, for example, chromosomal, non-chromosomal and synthetic polynucleotides, for example, derivatives of SV40, bacterial plasmids, phage DNAs, yeast plasmids, vectors derived from combinations of plasmids and phage DNAs, viral DNA such as vaccinia viruses, adenovirus, adeno-associated virus, lentivirus, canary pox virus, fowl pox virus, pseudorabies, baculovirus, herpes virus and retrovirus. The polynucleotide may be introduced into the expression vector by conventional procedures known in the art.

The polynucleotide which encodes one or more peptides may be operatively linked to an expression control sequence, i.e., a promoter, which directs mRNA synthesis. Representative examples of such promoters include the LTR or SV40 promoter, the E. coli lac or trp, the phage lambda PL promoter and other promoters known to control expression of genes in prokaryotic or eukaryotic cells or in viruses. The expression vector may also contain a ribosome binding site for translation initiation and a transcription terminator. The expression vectors may also include an origin of replication and a selectable marker, such as the ampicillin resistance gene of E. coli to permit selection of transformed cells, i.e., cells that are expressing the heterologous polynucleotide. The nucleic acid molecule encoding one or more of the peptides may be incorporated into the vector in frame with translation initiation and termination sequences.

One or more of the peptides can be recovered and purified from recombinant cell cultures (i.e., from the cells or culture medium) by well-known methods including ammonium sulphate or ethanol precipitation, acid extraction, anion or cation exchange chromatography, phosphocellulose chromatography, hydrophobic interaction chromatography, affinity chromatography, hydroxyapatite chromatography, lectin chromatography, and HPLC. Well known techniques for refolding proteins may be employed to regenerate active conformation when the peptide is denatured during isolation and or purification.

To produce a glycosylated peptide, it is preferred that recombinant techniques be used. To produce a glycosylated peptide, it is preferred that mammalian cells such as, COS-7 and Hep-G2 cells be employed in the recombinant techniques.

The peptides can also be prepared by cleavage of longer peptides, especially from food extracts.

Pharmaceutically acceptable salts of the peptides can be synthesised from the peptides which contain a basic or acid moiety by conventional chemical methods. Generally, the salts are prepared by reacting the free base or acid with stoichiometric amounts or with an excess of the desired salt-forming inorganic or organic acid or base in a suitable solvent.

### Gluten Challenge

In some embodiments, any one of the methods provided herein comprises a gluten challenge or a sample obtained from a subject before, during, or after a gluten challenge. Generally, a gluten challenge comprises administering to the subject a composition comprising wheat, rye, or barley, or one or more peptides thereof (e.g., a composition comprising a wheat gliadin, a rye secalin, or a barley hordein, or one or more peptides thereof), in some form for a defined period of time in order to activate the immune system of the subject, e.g., through activation of wheat-, rye- and/or barley-reactive T cells and/or mobilization of such T cells in the subject. Methods of gluten challenges are well known in the art and include oral, submucosal, supramucosal, and rectal administration of peptides or proteins (see, e.g., Can J Gastroenterol. 2001. 15(4):243-7. In vivo gluten challenge in celiac disease. Ellis H J, Ciclitira P J; Mol Diagn Ther. 2008. 12(5):289-98. Celiac disease: risk assessment, diagnosis, and monitoring. Setty M, Hormaza L. Guandalini S; Gastroenterology. 2009; 137(6):1912-33. Celiac disease: from pathogenesis to novel therapies. Schuppan D, Junker Y, Barisani D; J Dent Res. 2008; 87(12):1100-1107. Orally based diagnosis of celiac disease: current perspectives. Pastore L, Campisi G, Compilato D, and Lo Muzio L; Gastroenterology. 2001; 120:636-651. Current Approaches to Diagnosis and Treatment of Celiac Disease: An Evolving Spectrum. Fasano A and Catassi C; Clin Exp Immunol. 2000; 120:38-45. Local challenge of oral mucosa with gliadin in patients with coeliac disease. Lahteenoja M, Maki M, Viander M, Toivanen A, Syrjanen S; Clin Exp Immunol. 2000; 120:10-11. The mouth—an accessible region for gluten challenge. Ellis H and Ciclitira P; Clinical Science. 2001; 101:199-207. Diagnosing coeliac disease by rectal gluten challenge: a prospective study based on immunopathology, computerized image analysis and logistic regression analysis. Ensari A, Marsh M, Morgan S, Lobley R, Unsworth D, Kounali D, Crowe P, Paisley J, Moriarty K, and Lowry J; Gut. 2005; 54:1217-1223. T cells in peripheral blood after gluten challenge in coeliac disease. Anderson R, van Heel D. Tye-Din J. Barnardo M, Salio M, Jewell D, and Hill A; and Nature Medicine. 2000; 6(3):337-342. In vivo antigen challenge in celiac disease identifies a single transglutaminase-modified peptide as the dominant A-gliadin T-cell epitope. Anderson R, Degano P, Godkin A, Jewell D, and Hill A). Traditionally, a challenge lasts for several weeks (e.g., 4 weeks or more) and involves high doses of orally administered peptides or proteins (usually in the form of baked foodstuff that includes the peptides or proteins). Some studies suggest that a shorter challenge, e.g., through use of as little as 3 days of oral challenge, is sufficient to activate and/or mobilize reactive T-cells (Anderson R, van Heel D, Tye-Din J, Barnardo M, Salio M, Jewell D, and Hill A; and Nature Medicine. 2000; 6(3):337-342. In vivo antigen challenge in celiac disease identifies a single transglutaminase-modified peptide as the dominant A-gliadin T-cell epitope. Anderson R, Degano P, Godkin A, Jewell D, and Hill A). In some embodiments, any one of the methods provided herein comprises performing a gluten challenge on the subject or obtaining a sample from a subject before, during or after a gluten challenge, where the gluten challenge is for 6 weeks. In some embodiments, a gluten escalation (e.g., administering increasing amounts of gluten over time to a subject) is performed before the gluten challenge.

In some embodiments of any one of the methods provided herein, the challenge comprises administering a composition comprising wheat, barley and/or rye, or one or more peptides thereof. In some embodiments, the wheat is wheat flour, the barely is barley flour, and the rye is rye flour. In some embodiments, the challenge comprises administering a composition comprising a wheat gliadin, a barley hordein and/or a rye secalin, or one or more peptides thereof, to the subject prior to determining a T cell response as described herein.

In some embodiments of any one of the methods provided herein, the composition is administered to the subject after administration of a dose escalation regimen and a tolerizing regimen as described herein. In some embodiments, a sample is obtained from the subject after administration of the composition. In some embodiments, administration is for 6 weeks. In some embodiments, the composition contains 6 grams of gluten.

In some embodiments, administration is oral. Suitable forms of oral administration include foodstuffs (e.g., baked goods such as breads, cookies, cakes, etc.), tablets, troches, lozenges, aqueous or oily suspensions, dispersible powders or granules, emulsions, hard or soft capsules, or syrups or elixirs. Compositions intended for oral use may be prepared according to methods known to the art for the manufacture of pharmaceutical compositions or foodstuffs and such compositions may contain one or more agents including, for example, sweetening agents, flavoring agents, coloring agents and preserving agents in order to provide pharmaceutically elegant and palatable preparations.

In some embodiments, a sample is obtained from a subject before, during, and/or after a gluten challenge as described herein.

### Compositions, Vaccine Compositions, and Administration

### Compositions and Vaccine Compositions

The disclosure also provides a composition comprising at least one gluten peptide as provided herein. In some embodiments of any one of the compositions or methods provided, the composition comprises at least one peptide comprising at least one amino acid sequence selected from PFPQPELPY (SEQ ID NO: 4), PQPELPYPQ (SEQ ID NO: 5), PFPQPEQPF (SEQ ID NO: 6), PQPEQPFPW (SEQ ID NO: 7), PIPEQPQPY (SEQ ID NO: 8) and EQPIPEQPQ (SEQ ID NO: 9). In some embodiments of any one of the compositions or methods provided, the composition comprises at least one peptide selected from a first peptide comprising the amino acid sequence PFPQPELPY (SEQ ID NO: 4) and/or PQPELPYPQ (SEQ ID NO: 5); a second peptide comprising the amino acid sequence PFPQPEQPF (SEQ ID NO: 6) and/or PQPEQPFPW (SEQ ID NO: 7); and a third peptide comprising the amino acid sequence PIPEQPQPY (SEQ ID NO: 8) and/or EQPIPEQPQ (SEQ ID NO: 9). In some embodiments, the composition comprises a first peptide comprising the amino acid sequence ELQPFPQPELPYPQPQ (SEQ ID NO: 1), wherein the N-terminal glutamate is a pyroglutamate and the carboxyl group of the C-terminal glutamine is amidated; a second peptide comprising the amino acid sequence EQPFPQPEQPFPWQP (SEQ ID NO: 2), wherein the N-terminal glutamate is a pyroglutamate and the carboxyl group of the C-terminal proline is amidated; and a third peptide comprising the amino acid sequence EPEQPIPEQPQPYPQQ (SEQ ID NO: 3), wherein the N-terminal glutamate is a pyroglutamate and the carboxyl group of the C-terminal glutamine is amidated. In some embodiments, the composition is a vaccine composition.

As used herein, the term “vaccine” refers to a composition comprising one or more peptides that can be administered to a subject having Celiac disease to modulate the subject's response to gluten. The vaccine may reduce the immunological reactivity of a subject towards gluten. Preferably, the vaccine induces tolerance to gluten.

Without being bound by any theory, administration of the vaccine composition to a subject may induce tolerance by clonal deletion of gluten-specific effector T cell populations, for example, gluten-specific T cells, or by inactivation (anergy) of said T cells such that they become less responsive, preferably, unresponsive to subsequent exposure to gluten (or peptides thereof). Assessing immune tolerance, e.g., deletion or inactivation of said T cells can be measured, for example, by contacting ex vivo a sample comprising said T cells with gluten or a peptide thereof and measuring the response of said T cells to the gluten or peptide thereof. T cell response assays are known in the art (see, e.g., PCT Publication Number WO2010/060155).

Alternatively, or in addition, administration of the vaccine composition may modify the cytokine secretion profile of the subject (for example, result in decreased IL-4, IL-2, TNF-α and/or IFN-γ, and/or increased IL-10). The vaccine composition may induce suppressor T cell subpopulations, for example Treg cells, to produce IL-10 and/or TGF-β and thereby suppress gluten-specific effector T cells. The cytokine secretion profile of the subject can be measured using any method known to those of skill in the art, e.g., using immuno-based detection methods such as Western blot or enzyme-linked immunosorbent assay (ELISA).

The vaccine composition of the disclosure can be used for prophylactic treatment of a subject capable of developing Celiac disease and/or used in ongoing treatment of a subject who has Celiac disease. In some embodiments, the composition is for use in treating Celiac disease in a subject. In some embodiments, the subject is HLA-DQ2.5 positive. In some embodiments, the subject is HLA-DQ2.5 positive and HLA-DQ8 negative.

### Effective Amount

Compositions are generally administered in “effective amounts”. The term “effective amount” means the amount sufficient to provide the desired therapeutic or physiological effect when administered under appropriate or sufficient conditions. In some embodiments, the effective amount is an amount in micrograms of the peptides provided herein (i.e., the amount in micrograms/3 of the first peptide and an equimolar amount of each of the second and third peptides) or an equivalent, such as a molar equivalent thereof. In some embodiments, the effective amount is an amount (a nmol amount) of each of the first, second, and third peptides.

Methods for producing equimolar peptide compositions are known in the art and provided herein (see, e.g., Example 1 and Muller et al. Successful immunotherapy with T-cell epitope peptides of bee venom phospholipase A2 induces specific T-cell anergy in patient allergic to bee venom. J. Allergy Clin. Immunol. Vol. 101, Number 6. Part 1: 747-754 (1998)). In some embodiments, multiple effective dosages are utilized, e.g., to provide dose escalation. In some embodiments, one or more effective amounts of the peptides are administered in sterile sodium chloride 0.9% USP as a bolus intradermal injection.

The effective amounts provided herein, when used alone or in combination as part of a dosage schedule, are believed to modify the T cell response, e.g., by inducing immune tolerance, to wheat, barley and rye in the subject, and preferably wheat, barley, rye and oats. Thus, a subject treated according to the disclosure preferably is able to eat at least wheat, rye, barley and, optionally, oats without a significant T cell response which would normally lead to clinical manifestations of active Celiac disease.

### Pharmaceutically Acceptable Carriers

The compositions provided herein may include a pharmaceutically acceptable carrier. The term “pharmaceutically acceptable carrier” refers to molecular entities and compositions that do not produce an allergic, toxic or otherwise adverse reaction when administered to a subject, particularly a mammal, and more particularly a human. The pharmaceutically acceptable carrier may be solid or liquid. Useful examples of pharmaceutically acceptable carriers include, but are not limited to, diluents, excipients, solvents, surfactants, suspending agents, buffering agents, lubricating agents, adjuvants, vehicles, emulsifiers, absorbants, dispersion media, coatings, stabilizers, protective colloids, adhesives, thickeners, thixotropic agents, penetration agents, sequestering agents, isotonic and absorption delaying agents that do not affect the activity of the active agents of the disclosure. In some embodiments, the pharmaceutically acceptable carrier is a sodium chloride solution (e.g., sodium chloride 0.9% USP).

The carrier can be any of those conventionally used and is limited only by chemico-physical considerations, such as solubility and lack of reactivity with the active agent, and by the route of administration. Suitable carriers for this disclosure include those conventionally used, for example, water, saline, aqueous dextrose, lactose, Ringer's solution, a buffered solution, hyaluronan, glycols, starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like. Liposomes may also be used as carriers.

Techniques for preparing pharmaceutical compositions are generally known in the art as exemplified by Remington's Pharmaceutical Sciences, 16th Ed. Mack Publishing Company, 1980.

Administration preferably is intradermal administration. Thus, the composition(s) of the disclosure may be in a form suitable for intradermal injection. In some embodiments, the composition(s) of the disclosure are in the form of a bolus for intradermal injection.

### Injectables

The pharmaceutical composition(s) may be in the form of a sterile injectable aqueous or oleagenous suspension. In some embodiments, the composition is formulated as a sterile, injectable solution. This suspension or solution may be formulated according to known methods using those suitable dispersing or wetting agents and suspending agents which have been mentioned above. The sterile injectable preparation may be a suspension in a non-toxic parenterally-acceptable diluent or solvent, for example as a solution in 1,3-butanediol. Among the acceptable carriers that may be employed are water, Ringer's solution and isotonic sodium chloride solution. In some embodiments, the composition is formulated as a sterile, injectable solution, wherein the solution is a sodium chloride solution (e.g., sodium chloride 0.9% USP). In some embodiments, the composition is formulated as a bolus for intradermal injection.

Examples of appropriate delivery mechanisms for intradermal administration include, but are not limited to, implants, depots, needles, capsules, and osmotic pumps.

### Dosage

It is especially advantageous to formulate the active in a dosage unit form for ease of administration and uniformity of dosage. “Dosage unit form” as used herein refers to physically discrete units suited as unitary dosages for the subject to be treated; each unit containing a predetermined quantity of active agent calculated to produce the desired therapeutic effect in association with a pharmaceutical carrier. The specification for the dosage unit forms are dictated by and directly dependent on the unique characteristics of the active agent and the particular therapeutic effect to be achieved, and the limitations inherent in the art of compounding such an active agent for the treatment of subjects. Examples of dosage units include sealed ampoules and vials and may be stored in a freeze-dried condition requiring only the addition of the sterile liquid carrier immediately prior to use.

The composition(s) may also be included in a container, pack, or dispenser together with instructions for administration.

The actual amount(s) administered (or dose or dosage) and the rate and time-course of administration are as provided herein in any one of the methods provided.

The administration of any one of the methods provided may occur at least once, twice or three times a week. In some embodiments of any one of the methods provided, a composition described herein is administered twice a week. In some embodiments of any one of the methods provided, a composition described herein is administered for at least 6, 7, 8, 9 or 10 weeks. In some embodiments of any one of the methods provided, a composition described herein is administered twice a week for 8 weeks. In some embodiments of any one of the methods provided, a dose escalation phase can last for at least 3, 4, 5, 6, 7, 8, 9 or 10 weeks with the dosings occurring at any one of the intervals provided herein. In some embodiments of any one of the methods provided, a tolerizing phase can last for at least 3, 4, 5, 6, 7, 8, 9 or 10 weeks with the dosings occurring at any one of the intervals provided herein.

In some embodiments, the frequency of administration (and/or the dosage) may change, depending on the phase of treatment (e.g., a dose escalation phase or a tolerizing phase).

In some embodiments, during a tolerizing phase, at least 150, 175, 200, 225, 250, 275, 300, 325, 350, 375, 400, 425, 450, 475, 500, 525, 550, 575, 600, 625, 650, 675, 700, 725, 750, 775, 800, 825, 850, 875 or 900 micrograms (or an equivalent, such as a molar equivalent, thereof) of the peptides described herein (e.g., second composition) are administered. The administration can be according to any one of the intervals and can last according to any one of the time periods provided herein.

In some embodiments, during a tolerizing phase, a subject, such as one having a non-homozygous HLA-DQ2.5 genotype, is administered at least 300, 325, 350, 375, 400, 425, 450, 475, 500, 525, 550, 575, 600, 625, 650, 675, 700, 725, 750, 775, 800, 825, 850, 875 or 900 micrograms (or an equivalent, such as a molar equivalent, thereof) of the peptides described herein (e.g., second composition).

In some embodiments, any one of the treatment methods described herein comprises any one of the tolerizing phases provided herein and any one of the dose escalation phases provided herein (preferably, prior to the tolerizing phase, in some embodiments).

### Kits

Another aspect of the disclosure relates to kits. In some embodiments, the kit comprises one or more compositions comprising the peptides as described herein. In some embodiments, the kit comprises at least two compositions at at least two different effective amounts described herein. In some embodiments a kit is provided that comprises gluten peptide compositions at each of the doses of any one of the methods provided herein.

In some embodiments of any one of the kits described, the one or more gluten peptides are a first peptide comprising the amino acid sequence PFPQPELPY (SEQ ID NO: 4) and/or PQPELPYPQ (SEQ ID NO: 5); a second peptide comprising the amino acid sequence PFPQPEQPF (SEQ ID NO: 6) and/or PQPEQPFPW (SEQ ID NO: 7); and a third peptide comprising the amino acid sequence PIPEQPQPY (SEQ ID NO: 8) and/or EQPIPEQPQ (SEQ ID NO: 9). In some embodiments of any one of the kits described, one or more gluten peptides are a first peptide comprising the amino acid sequence ELQPFPQPELPYPQPQ (SEQ ID NO: 1), wherein the N-terminal glutamate is a pyroglutamate and the carboxyl group of the C-terminal glutamine is amidated; a second peptide comprising the amino acid sequence EQPFPQPEQPFPWQP (SEQ ID NO: 2), wherein the N-terminal glutamate is a pyroglutamate and the carboxyl group of the C-terminal proline is amidated; and a third peptide comprising the amino acid sequence EPEQPIPEQPQPYPQQ (SEQ ID NO: 3), wherein the N-terminal glutamate is a pyroglutamate and the carboxyl group of the C-terminal glutamine is amidated.

In some embodiments of any one of the kits described, the kit comprises compositions for any one of the tolerizing phases provided herein and any one of the dose escalation phases provided herein. The peptides can be contained within the same container or separate containers. In some embodiments of any one of the kits described, the peptide or peptides may be contained within the container(s) (e.g., dried onto the wall of the container(s)). In some embodiments of any one of the kits described, the peptides are contained within a solution separate from the container, such that the peptides may be added to the container at a subsequent time. In some embodiments of any one of the kits described, the peptides are in lyophilized form in a separate container, such that the peptides may be reconstituted and added to another container at a subsequent time. In some embodiments of any one of the kits described, the one or more compositions comprised within the kit are in a container that is suitable for intradermal injection (e.g., a device containing a needle such as a syringe). In some embodiments of any one of the kits described, the kit comprises a container that is suitable for intradermal injection (e.g., a device containing a needle such as a syringe).

In some embodiments of any one of the kits described, the kit further comprises instructions for reconstitution, mixing, administration, etc. In some embodiments of any one of the kits described, the instructions include the methods described herein. Instructions can be in any suitable form, e.g., as a printed insert or a label.

### Methods of Treatment

Aspects of the disclosure relate to use of the compositions described herein for treating a subject having, suspected of having or at risk of having Celiac disease.

As used herein, the terms “treat”, “treating”, and “treatment” include abrogating, inhibiting, slowing, or reversing the progression of a disease or condition, or ameliorating or preventing a clinical symptom of the disease (for example, Celiac disease). Treatment may include induction of immune tolerance (for example, to gluten or peptides thereof), modification of the cytokine secretion profile of the subject and/or induction of suppressor T cell subpopulations to secrete cytokines. Thus, a subject treated according to the disclosure preferably is able to eat at least wheat, rye, barley and, optionally, oats without a significant T cell response which would normally lead to symptoms of Celiac disease.

“Administering” provided herein include direct administration of a composition provided herein as well as indirect administration such as a clinician directing a subject to administer the composition.

### Identifying Subjects for Treatment

In some embodiments, methods described herein comprise treating a subject who has Celiac disease. Thus, it may be desirable to identify subjects, such as subjects with Celiac disease, who are likely to benefit from administration of a composition described herein. It may also be desirable to monitor the treatment of the subjects with the compositions and methods provided herein. Any diagnostic method or other assay or combinations thereof are contemplated for identifying or monitoring such a subject. Any one of the methods provided herein can include identification and/or monitoring step(s). Exemplary methods include, but are not limited to, intestinal biopsy, serology (measuring the levels of one or more antibodies present in the scrum), and genotyping (see, e.g., Husby S, Kolctzko S, Korponay-Szabo I R, Mearin M L, Phillips A. Shamir R, Troncone R, Giersiepen K, Branski D, Catassi C et al: European Society for Pediatric Gastroenterology, Hepatology, and Nutrition guidelines for the diagnosis of coeliac disease. J Pediatr Gastroenterol Nutr 2012, 54(1):136-160. AND/OR Rubio-Tapia A, Hill I D, Kelly C P, Calderwood A H, Murray J A. ACG clinical guidelines: diagnosis and management of celiac disease. Am J Gastroenterol 2013; 108:656-76. AND/OR Ludvigsson J F, Leffler D A, Bai J C, Biagi F, Fasano A, Green P H, Hadjivassiliou M, Kaukinen K, Kelly C P, Leonard J N, Lundin K E, Murray J A, Sanders D S, Walker M M, Zingone F, Ciacci C. The Oslo definitions for coeliac disease and related terms. Gut 2012; 62:43-52.).

The presence of serum antibodies can be detected using methods known to those of skill in the art, e.g., by ELISA, histology, cytology, immunofluorescence or western blotting. Such antibodies include, but are not limited to: IgA anti-endomysial antibody (IgA EMA), IgA anti-tissue transglutaminase 2 antibody (IgA tTG), IgA anti-deamidated gliadin peptide antibody (IgA DGP), and IgG anti-deamidated gliadin peptide antibody (IgG DGP). Deamidated gliadin peptide-IgA (DGP-IgA) and deamidated gliadin peptide-IgG (DGP-IgG) can be evaluated with commercial kits (e.g. INV 708760, 704525, and 704520, INOVA Diagnostics, San Diego, Calif.).

Subjects can be tested for the presence of the HLA-DQA and HLA-DQB susceptibility alleles encoding HLA-DQ2.5 (DQA1*05 and DQB1*02), DQ2.2 (DQA1*02 and DQB1*02) or DQ8 (DQA1*03 and DQB1*0302). Exemplary sequences that encode the DQA and DQB susceptibility alleles include HLA-DQA1*0501 (Genbank accession number: AF515813.1) HLA-DQA1*0505 (AH013295.2), HLA-DQB1*0201 (AY375842.1) or HLA-DQB1*0202 (AY375844.1). Methods of genetic testing are well known in the art (see, e.g., Bunce M, et al. Phototyping: comprehensive DNA typing for HLA-A, B, C, DRB1, DRB3, DRB4. DRB5 & DQB1 by PCR with 144 primer mixes utilizing sequence-specific primers (PCR-SSP). Tissue Antigens 46, 355-367 (1995); Olerup O, Aldener A, Fogdell A. HLA-DQB1 and DQA1 typing by PCR amplification with sequence-specific primers in 2 hours. Tissue antigens 41, 119-134 (1993); Mullighan C G, Bunce M, Welsh K I. High-resolution HLA-DQB1 typing using the polymerase chain reaction and sequence-specific primers. Tissue-Antigens. 50, 688-92 (1997); Koskinen L, Romanos J, Kaukinen K, Mustalahti K, Korponay-Szabo I, et al. (2009) Cost-effective HLA typing with tagging SNPs predicts celiac disease risk haplotypes in the Finnish, Hungarian, and Italian populations. Immunogenetics 61: 247-256; and Monsuur A J, de Bakker P I, Zhernakova A, Pinto D, Verduijn W, et al. (2008) Effective detection of human leukocyte antigen risk alleles in celiac disease using tag single nucleotide polymorphisms. PLoS ONE 3: e2270). Subjects that have one or more copies of a susceptibility allele are considered to be positive for that allele. Detection of the presence of susceptibility alleles can be accomplished by any nucleic acid assay known in the art, e.g., by polymerase chain reaction (PCR) amplification of DNA extracted from the patient followed by hybridization with sequence-specific oligonucleotide probes or using leukocyte-derived DNA (Koskinen L, Romanos J, Kaukinen K, Mustalahti K, Korponay-Szabo I, Barisani D. Bardella M T, Ziberna F, Vatta S, Szeles G et al: Cost-effective HLA typing with tagging SNPs predicts Celiac disease risk haplotypes in the Finnish, Hungarian, and Italian populations. Immunogenetics 2009, 61(4):247-256; Monsuur A J, de Bakker P I, Zhernakova A, Pinto D, Verduijn W, Romanos J, Auricchio R, Lopez A, van Heel D A, Crusius J B et al: Effective detection of human leukocyte antigen risk alleles in Celiac disease using tag single nucleotide polymorphisms. PLoS ONE 2008, 3(5):e2270).

### EXEMPLARY EMBODIMENTS

The following are additional, non-limiting example embodiments of the disclosure.

Clause 1. A method for treating Celiac disease in a subject, the method comprising: administering to the subject a dose escalation regimen of a gluten peptide composition comprising a first, second and third peptide, wherein the dose escalation regimen comprises administering the following doses sequentially and at least one day apart from each other: 1, 3, 6, 9, 30, 60, 90, 150, 300, 450, 600 and 750 micrograms of the gluten peptide composition; and subsequently administering to the subject during a tolerizing regimen a dose of 900 micrograms of the gluten peptide composition, wherein:
the first peptide comprises the amino acid sequence ELQPFPQPELPYPQPQ (SEQ ID NO: 1), wherein the N-terminal glutamate is a pyroglutamate and the C-terminal glutamine is amidated;
the second peptide comprises the amino acid sequence EQPFPQPEQPFPWQP (SEQ ID NO: 2), wherein the N-terminal glutamate is a pyroglutamate and the C-terminal proline is amidated; and
the third peptide comprises the amino acid sequence EPEQPIPEQPQPYPQQ (SEQ ID NO: 3), wherein the N-terminal glutamate is a pyroglutamate and the C-terminal glutamine is amidated.
   Clause 2. The method of clause 1, wherein the doses in the dose escalation regimen are administered to the subject two times per week, with each dose administered between one to three times before escalation to the next highest dose.
   Clause 3. The method of clause 1 or 2, wherein the 900 microgram dose in the tolerizing regimen is administered to the subject two times per week.
   Clause 4. The method of any one of clauses 1 to 3, wherein:

the 1 microgram dose contains one third of a microgram of the first peptide and an equimolar amount of each of the second and third peptides;

the 3 microgram dose contains 1 microgram of the first peptide and an equimolar amount of each of the second and third peptides;

the 6 microgram dose contains 2 micrograms of the first peptide and an equimolar amount of each of the second and third peptides;

the 9 microgram dose contains 3 micrograms of the first peptide and an equimolar amount of each of the second and third peptides;

the 30 microgram dose contains 10 micrograms of the first peptide and an equimolar amount of each of the second and third peptides;

the 60 microgram dose contains 20 micrograms of the first peptide and an equimolar amount of each of the second and third peptides;

the 90 microgram dose contains 30 micrograms of the first peptide and an equimolar amount of each of the second and third peptides;

the 150 microgram dose contains 50 micrograms of the first peptide and an equimolar amount of each of the second and third peptides;

the 300 microgram dose contains 100 micrograms of the first peptide and an equimolar amount of each of the second and third peptides;

the 450 microgram dose contains 150 micrograms of the first peptide and an equimolar amount of each of the second and third peptides;

the 600 microgram dose contains 200 micrograms of the first peptide and an equimolar amount of each of the second and third peptides;

the 750 microgram dose contains 250 micrograms of the first peptide and an equimolar amount of each of the second and third peptides; and

the 900 microgram dose contains 300 micrograms of the first peptide and an equimolar amount of each of the second and third peptides.

Clause 5. The method of any one of clauses 1 to 4, wherein at least one dose of the tolerizing regimen is self-administered by the patient.
Clause 6. The method of any one of clauses 1 to 5, wherein each of the gluten peptide compositions are administered subcutaneously.
Clause 7. The method of any one of clauses 1 to 6, wherein each of the gluten peptide compositions are formulated as a sterile, injectable solution.
Clause 8. The method of clause 7, wherein the sterile, injectable solution is sodium chloride.
Clause 9. The method of clause 8, wherein the sodium chloride is sterile sodium chloride 0.9% USP.
Clause 10. A method for treating Celiac disease in a subject, the method comprising: administering to the subject at least two different gluten peptide compositions (i.e., each with a different amount of the gluten peptides) during a dose escalation phase, wherein each gluten peptide composition comprises less than 150 micrograms gluten peptide (e.g., 50 micrograms of a first peptide and an equimolar amount of each of a second and a third peptide); and subsequently administering to the subject during a tolerizing phase a second composition comprising at least 150, 160, 170, 180, 190, 200, 210, 220, 230, 240, 250, 260, 270, 280, 290, or 300 micrograms gluten peptide (e.g., 100 micrograms of the first peptide and an equimolar amount of each of the second and third peptides), wherein:
the first peptide comprises the amino acid sequence ELQPFPQPELPYPQPQ (SEQ ID NO: 1), wherein the N-terminal glutamate is a pyroglutamate and the C-terminal glutamine is amidated;
the second peptide comprises the amino acid sequence EQPFPQPEQPFPWQP (SEQ ID NO: 2), wherein the N-terminal glutamate is a pyroglutamate and the C-terminal proline is amidated; and
the third peptide comprises the amino acid sequence EPEQPIPEQPQPYPQQ (SEQ ID NO: 3), wherein the N-terminal glutamate is a pyroglutamate and the C-terminal glutamine is amidated, and
   optionally, wherein at least one or all of the gluten peptide compositions of the dose escalation phase is in an amount different from any of 3, 6, 9, 30, 60, 90, and 150 micrograms of the gluten peptides.
   Clause 11. The method of clause 10, wherein the at least two different gluten peptide compositions administered during the dose escalation phase are at least 3, 4, 5, 6, 7, 8, 9 or 10 different gluten peptide compositions.
   Clause 12. The method of clause 10 or 11, wherein each of the at least two different gluten peptide compositions is in an amount of 1 to 149 (i.e., 1, 2, 3, 4, 5, . . . 145, 146, 147, 148 or 149, including any integer between 5 and 145) micrograms, with each different gluten peptide composition administered subsequent is in an amount greater than the previous administered different gluten peptide composition.
   Clause 13. The method of any one of the preceding clauses, wherein the at least two different gluten peptide compositions of the dose escalation phase comprise a first gluten peptide composition in an amount between 1 and 10 micrograms.
   Clause 14. The method of clause 13, wherein the at least two different gluten peptide compositions of the dose escalation phase comprise a second gluten peptide composition in an amount between 10 and 75 micrograms.
   Clause 15. The method of clause 14, wherein the at least two different gluten peptide compositions of the dose escalation phase comprise a third gluten peptide composition in an amount between 50 and 100 micrograms.
   Clause 16. The method of clause 15, wherein the at least two different gluten peptide compositions of the dose escalation phase comprise a fourth gluten peptide composition in an amount between 75 and 149 micrograms.
   Clause 17. The method of clause 13 or 14, wherein the first and/or second gluten peptide composition is administered once or twice.
   Clause 18. The method of any one of clauses 15-17, wherein the third and/or fourth gluten peptide composition is administered at least twice.
   Clause 19. The method of any one of the preceding clauses, wherein the dose escalation period is at least 3, 3.5, 4, 4.5, 5, 5.5, 6, 6.5, 7, 7.5, 8 or more weeks.
   Clause 20. The method of any one of the preceding clauses, wherein the tolerizing phase is at least 3, 3.5, 4, 4.5, 5, 5.5, 6, 6.5, 7, 7.5, 8 or more weeks.
   Clause 21. The method of any one of the preceding clauses, wherein the subject has a homozygous HLA-DQ2.5 genotype.
   Clause 22. A method for treating Celiac disease in a subject, the method comprising: administering to the subject at least two different gluten peptide compositions (i.e., each with a different amount of the gluten peptides) during a dose escalation phase, wherein each gluten peptide composition comprises less than 900 micrograms gluten peptide (e.g., 300 micrograms of a first peptide and an equimolar amount of each of a second and a third peptide); and subsequently administering to the subject during a tolerizing phase a second composition comprising at least 500, 550, 600, 650, 700, 750, 800, 850, or 900 micrograms gluten peptide (e.g., 300 micrograms of the first peptide and an equimolar amount of each of the second and third peptides), wherein:
the first peptide comprises the amino acid sequence ELQPFPQPELPYPQPQ (SEQ ID NO: 1), wherein the N-terminal glutamate is a pyroglutamate and the C-terminal glutamine is amidated;
the second peptide comprises the amino acid sequence EQPFPQPEQPFPWQP (SEQ ID NO: 2), wherein the N-terminal glutamate is a pyroglutamate and the C-terminal proline is amidated; and the third peptide comprises the amino acid sequence EPEQPIPEQPQPYPQQ (SEQ ID NO: 3), wherein the N-terminal glutamate is a pyroglutamate and the C-terminal glutamine is amidated, and
   optionally, wherein at least one or all of the gluten peptide composition of the dose escalation phase is in an amount different from any of 3, 6, 9, 30, 60, 90, 150, 300, 450, 600 and 750 micrograms of the gluten peptides.
   Clause 23. The method of clause 24, wherein the at least two different gluten peptide compositions administered during the dose escalation phase are at least 3, 4, 5, 6, 7, 8, 9, 10, 11, 12, 13, 14, or 15 different gluten peptide compositions.
   Clause 24. The method of clause 22 or 23, wherein each of the at least two different gluten peptide compositions is in an amount of 1 to 899 (i.e., 1, 2, 3, 4, 5, . . . 895, 896, 897, 898 or 899, including any integer between 5 and 895) micrograms, with each different gluten peptide composition administered subsequent is in an amount greater than the previous administered different gluten peptide composition.
   Clause 25. The method of any one of clauses 22-24, wherein the at least two different gluten peptide compositions of the dose escalation phase comprise a first gluten peptide composition in an amount between 1 and 10 micrograms.
   Clause 26. The method of clause 25, wherein the at least two different gluten peptide compositions of the dose escalation phase comprise a second gluten peptide composition in an amount between 10 and 75 micrograms.
   Clause 27. The method of clause 26, wherein the at least two different gluten peptide compositions of the dose escalation phase comprise a third gluten peptide composition in an amount between 50 and 100 micrograms.
   Clause 28. The method of clause 27, wherein the at least two different gluten peptide compositions of the dose escalation phase comprise a fourth gluten peptide composition in an amount between 75 and 150 micrograms.
   Clause 29. The method of clause 28, wherein the at least two different gluten peptide compositions of the dose escalation phase comprise a fifth gluten peptide composition in an amount between 100 and 300 micrograms.
   Clause 30. The method of clause 29, wherein the at least two different gluten peptide compositions of the dose escalation phase comprise a sixth gluten peptide composition in an amount between 150 and 500 micrograms.
   Clause 31. The method of clause 30, wherein the at least two different gluten peptide compositions of the dose escalation phase comprise a seventh gluten peptide composition in an amount between 300 and 750 micrograms.
   Clause 32. The method of clause 31, wherein the at least two different gluten peptide compositions of the dose escalation phase comprise a eighth gluten peptide composition in an amount between 500 and 899 micrograms.
   Clause 33. The method of any one of clauses 25-27, wherein the first, second and/or third gluten peptide composition is administered once or twice.
   Clause 34. The method of any one of clauses 27-33, wherein the third, fourth, fifth, sixth, seventh and/or eighth gluten peptide composition is administered at least twice.
   Clause 35. The method of any one of clauses 22-34, wherein the dose escalation period is at least 3, 3.5, 4, 4.5, 5, 5.5, 6, 6.5, 7, 7.5, 8 or more weeks.
   Clause 36. The method of any one of clauses 22-35, wherein the tolerizing phase is at least 3, 3.5, 4, 4.5, 5, 5.5, 6, 6.5, 7, 7.5, 8 or more weeks.
   Clause 37. The method of any one of clauses 22-36, wherein the subject has a non-homozygous HLA-DQ2.5 genotype.
   Clause 38. The method of any one of the preceding clauses, wherein the dose escalation phase includes a gluten peptide composition that is administered that comprises an amount of 1 microgram gluten peptides.
   Clause 39. The method of any one of the preceding clauses, wherein the first gluten peptide composition comprises an amount of 1 microgram gluten peptide.
   Clause 40. The method of any one of the preceding clauses, wherein the gluten peptide compositions of the dose escalation and/or tolerizing phase(s) is/are administered twice a week.
   Clause 41. The method of any one of the preceding clauses, wherein the time between gluten peptide composition administrations of the dose escalation and/or tolerizing phase(s) is 1, 2, 3, 4, 5 or more day(s).
   Clause 42. The method of any one of the preceding clauses, wherein each of the gluten peptide compositions are administered intradermally.
   Clause 43. The method of any one of the preceding clauses, wherein each of the gluten peptide compositions are administered subcutaneously.
   Clause 44. The method of any of the preceding clauses, wherein each of the gluten peptide compositions are formulated as a sterile, injectable solution.
   Clause 45. The method of clause 44, wherein the sterile, injectable solution is sodium chloride.
   Clause 46. The method of clause 45, wherein the sodium chloride is sterile sodium chloride 0.9% USP.
   Clause 47. The method of any one of the preceding clauses, wherein the subject is any one of the subjects provided herein.
   Clause 48. A method for treating Celiac disease in a subject, the method comprising administering one or more gluten peptide compositions according to any one of the dosing regimens provided herein, such as in the Examples or Figures.
   Clause 49. A method for treating Celiac disease in a subject, the method comprising administering one or more gluten peptide compositions according to any one of the titration or dose escalation regimens or phases as provided herein and any one of the tolerizing or maintenance regimens or phases as provided herein, such as in any one of the Examples or Figures.
   Clause 50. The method of clause 48 or 49, wherein the one or more gluten peptide compositions comprises any one of the gluten peptide compositions provided herein.
   Clause 51. The method of clause 50, wherein the one or more gluten peptide compositions comprises peptides 1, 2 and 3 of Example 6.
   Clause 52. The method of any one of clauses 48-51, wherein the subject is any one of the subjects provided herein.
   Clause 53. The method of any one of clauses 48-52, wherein the dose escalation regimen or phase further comprises a dose of a gluten peptide composition in an amount of 1 microgram gluten peptide.
   Clause 54. The method of any one of clauses 48-53, wherein the dose escalation regimen or phase comprises the administration of different gluten peptide compositions, the gluten peptide compositions, respectively, comprising 1, 3, 9, 30, 60, 90 and 150 micrograms gluten peptide.
   Clause 55. The method of clause 54, wherein the doses of gluten peptide compositions of the dose escalation phase are administered according to any one of the intervals and frequencies provided herein.
   Clause 56. The method of clause 54 or 55, wherein the gluten peptide composition of the tolerizing phase comprises any one of the gluten peptide compositions of the tolerizing phase provided herein, such as at least 300 micrograms gluten peptide.
   Clause 57. The method of any one of clauses 54-56, wherein the gluten peptide composition of the tolerizing phase is given according to any one of the intervals or frequencies provided herein.
   Clause 58. The method of any one of clauses 54-57, wherein the subject is a homozygous HLA-DQ2.5 genotype.
   Clause 59. The method of any one of clauses 48-53, wherein the dose escalation regimen or phase comprises the administration of different gluten peptide compositions, the gluten peptide compositions, respectively, comprising 1, 3, 9, 30, 60, 90, 150, 300, 450, 600 and 750 micrograms gluten peptide.
   Clause 60. The method of clause 59, wherein the doses of gluten peptide compositions of the dose escalation phase are administered according to any one of the intervals and frequencies provided herein.
   Clause 61. The method of clause 59 or 60, wherein the gluten peptide composition of the tolerizing phase comprises any one of the gluten peptide compositions of the tolerizing phase provided herein, such as at least 900 micrograms gluten peptide.
   Clause 62. The method of any one of clauses 59-61 wherein the gluten peptide composition of the tolerizing phase is given according to any one of the intervals or frequencies provided herein.
   Clause 63. The method of any one of clauses 59-62, wherein the subject is a non-homozygous HLA-DQ2.5 genotype.
   Clause 64. The method of any one of clauses 57-63, wherein each of the gluten peptide compositions are administered subcutaneously.
   Clause 65. The method of any one of clauses 57-63, wherein each of the gluten peptide compositions are formulated as a sterile, injectable solution.
   Clause 66. The method of clause 65, wherein the sterile, injectable solution is sodium chloride.
   Clause 67. The method of clause 66, wherein the sodium chloride is sterile sodium chloride 0.9% USP.
   Clause 68. One or more gluten peptide compositions for performing a method as in any one of the preceding clauses.
   Clause 69. A kit comprising one or more gluten peptide compositions for performing a method as in any one of the preceding clauses.

### EXAMPLES

### Example 1: Preparation of a 150 Microgram Dosage Composition of the First, Second, and Third Peptide

A peptide composition contains three peptides as shown below (the “peptide composition,” in its various doses described herein, in some instances, is also referred to herein as Nexvax2):

| Peptide | | T-cell epitopes |
|---|---|---|
| Number | Sequence | contained in the peptide |
| 1 (also referred | (pE)LQPFPQPELPYPQPQ-NH2 | PFPQPELPY (SEQ ID NO: 4), |
| to as NPL001) | (SEQ ID NO: 10) | PQPELPYPQ (SEQ ID NO: 5) |
| 2 (also referred | (pE)QPFPQPEQPFPWQP-NH2 | PFPQPEQPF (SEQ ID NO: 6), |
| to as NPL002) | (SEQ ID NO: 11) | PQPEQPFPW (SEQ ID NO: 7) |
| 3 (also referred | (pE)PEQPEIPQPQPYPQQ-NH2 | PIPEQPQPY (SEQ ID NO: 8), |
| to as NPL003) | (SEQ ID NO: 12) | EQPIPEQPQ (SEQ ID NO: 9) |

A dose of 150 μg the peptide composition was defined by there being 50 μg (26.5 nmol) of pure peptide 1, and an equimolar amount of peptide 2 and peptide 3. The molar equivalent of 50 μg peptide 1 was given by 50 μg/1889.3 g/mol=26.5 nmol. When preparing a solution containing 150 μg of the peptide composition, for the constituent peptides, the weight of each peptide was adjusted according to peptide purity and peptide content of the lyophilized stock material. For example, if the peptide 1 stock material had peptide purity of 98% and its peptide content was 90%, the weight of stock material yielding 50 μg peptide 1 was 50 μg/(peptide purity×peptide content)=50 ug/(0.98×0.90)=56.7 ug.

The molar amount of peptide 1 in 150 Kg of the peptide composition was 26.5 nmol, and the weight of lyophilized peptide 2 stock material was therefore given by 26.5 nmol×1833.2 g/mol/(peptide purity×peptide content). For example, if peptide 2 peptide purity was 99%, and peptide content of 95%, the mass of stock required was 51.7 ug.

The molar amount of peptide 3 in 150 ug of the peptide composition was 26.5 nmol, and the weight of lyophilized peptide 3 stock material was therefore given by 26.5 nmol×1886.2 g/mol/(peptide purity×peptide content). For example, if peptide 3 peptide purity was 98%, and peptide content of 92%, the mass of stock required was 55.4 ug.

0.9, 3, 9, 30, and 90 or any of the other microgram dosage compositions provided herein can be prepared similarly.

### Example 2: Dose Escalation Study

Objective: Determine tolerability of different escalating regimens followed by fixed dose and schedule for tolerance induction. Reduce adverse events and cytokine elevation associated with a large 1 time bolus (150 mcg) of peptide composition.

### Key Inclusion/Exclusion

patients having Celiac disease that are HLA-DQ2.5+

### Study Design

36 patients having Celiac disease that are HLA-DQ2.5+
Patients are administered doses of the peptide composition comprising peptide 1, 2, and 3 described herein (a first peptide comprising the amino acid sequence ELQPFPQPELPYPQPQ (SEQ ID NO: 1), wherein the N-terminal glutamate is a pyroglutamate and the carboxyl group of the C-terminal glutamine is amidated; a second peptide comprising the amino acid sequence EQPFPQPEQPFPWQP (SEQ ID NO: 2), wherein the N-terminal glutamate is a pyroglutamate and the carboxyl group of the C-terminal proline is amidated; and a third peptide comprising the amino acid sequence EPEQPIPEQPQPYPQQ (SEQ ID NO: 3), wherein the N-terminal glutamate is a pyroglutamate and the carboxyl group of the C-terminal glutamine is amidated or placebo on the following dosage schedule:
Dose escalation regimen (or phase) for 5 doses at 0.9, 3, 9, 30, and 90 micrograms or placebo 2× a week for 2.5 weeks
Tolerizing regimen (or phase) of 150 micrograms twice a week for 8 weeks, follows dose escalation regimen

### Key Assessments

Primary Endpoint: Cytokine secretion
Secondary Endpoint: Symptoms

### Example 3. Further Dose Escalation Study Design

Primary Objective: To compare quantitative duodenal histology after a six week gluten challenge in HLA-DQ2.5+ patients with celiac disease on a gluten-free diet (GFD) who have been administered the peptide composition in Example 1 or placebo by intradermal injection.

Secondary Objective: To compare symptoms during a six week gluten challenge in HLA-DQ2.5+ patients with celiac disease on a gluten-free diet (GFD) who have been administered the peptide composition in Example 1 or placebo by intra-dermal injection.

### Study Design

The dose escalation regimen (or phase) and tolerizing regimen (or phase) described in Example 1 are carried out. A gluten escalation is performed over 14 days, followed by a 6 gram gluten challenge over 6 weeks. A biopsy is performed before the gluten escalation and after the 6 week challenge.

### Endpoints

Primary: VH:CrD—before vs after gluten challenge
Secondary: Clinical symptoms averaged for the last 2 weeks of subjects gluten challenge

### Example 4. Further Dose Escalation Study

### Primary Endpoint

Safety and tolerability

### Secondary Endpoint

Weekly GI symptoms per Gastrointestinal System Rating Scale
Assessment of plasma cytokine levels after sequential doses of gluten peptide composition

### Patients

Biopsy-confirmed, DQ2.5+ celiac disease patients on a GFD

### Dosing:

Titration Phase
   dose titration regimen to 300 micrograms for 2 weeks (3, 9, 30, 60, 90, 150, and 300 micrograms) or placebo
Tolerizing Phase
   dose at 300 micrograms twice per week or placebo for 4 weeks
Follow-up Phase
   4 weeks of follow up

### Example 5. Dose Escalation Study in Non-Homozygotes for DQ2.5+

### Dosing:

Titration Phase
   dose titration regimen up to 900 micrograms for 4.5 weeks (3, 9, 30, 60, 90, 150, 300, 450, 600, 750, and up to 900 micrograms) or placebo
Maintenance Dosing Phase
   Dose at 300 micrograms (or up to 900 micrograms) or placebo twice per week for 4 weeks
Follow-up Phase
   4 weeks of follow up

### Example 6. Dose Escalation Study and Results

### Primary Endpoint

Treatment emergent adverse events (TEAEs)

### Secondary Endpoints

Weekly Gastrointestinal Symptom Rating Scale (GSRS) scores, and relative change in plasma cytokine levels 4 hours after 150 microgram and higher doses. Plasma concentrations pre- and 45 min post-dose, and villous height to crypt depth ratio (VH:CrD) in 2nd part duodenal biopsies were assessed in Cohort 3.

### Patients

Biopsy-confirmed, DQ2.5+ celiac disease patients on a gluten-free diet

Patients were administered doses of peptide composition comprising peptide 1, 2, and 3 described herein (a first peptide comprising the amino acid sequence ELQPFPQPELPYPQPQ (SEQ ID NO: 1), wherein the N-terminal glutamate is a pyroglutamate and the carboxyl group of the C-terminal glutamine is amidated; a second peptide comprising the amino acid sequence EQPFPQPEQPFPWQP (SEQ ID NO: 2), wherein the N-terminal glutamate is a pyroglutamate and the carboxyl group of the C-terminal proline is amidated; and a third peptide comprising the amino acid sequence EPEQPIPEQPQPYPQQ (SEQ ID NO: 3), wherein the N-terminal glutamate is a pyroglutamate and the carboxyl group of the C-terminal glutamine is amidated) or placebo on the following dosage schedule.

### Dosing Regimen:

Cohorts 1 & 2 Titration Phase
   Twice-weekly dosing
   Initial up-dosing regimen of 30, 60, 90, 150, and 300 micrograms of peptide composition (or placebo)
   Amended to 3, 9, 30, 60, 90, 150, and 300 micrograms of peptide composition (or placebo)
Cohort 3 Titration Phase
   Dose titration regimen up to 900 micrograms of peptide composition for 4.5 weeks (3, 9, 30, 60, 90, 150, 300, 450, 600, 750, and up to 900 micrograms) (or placebo)
Maintenance Dosing Phase
   Cohorts 1 & 2: dose at 300 micrograms of peptide composition twice per week for 4 weeks (or placebo)
   Cohort 3: dose at maximum tolerated dose up to 900 micrograms of peptide composition (or placebo)
Follow-up Phase
   4 weeks of follow up

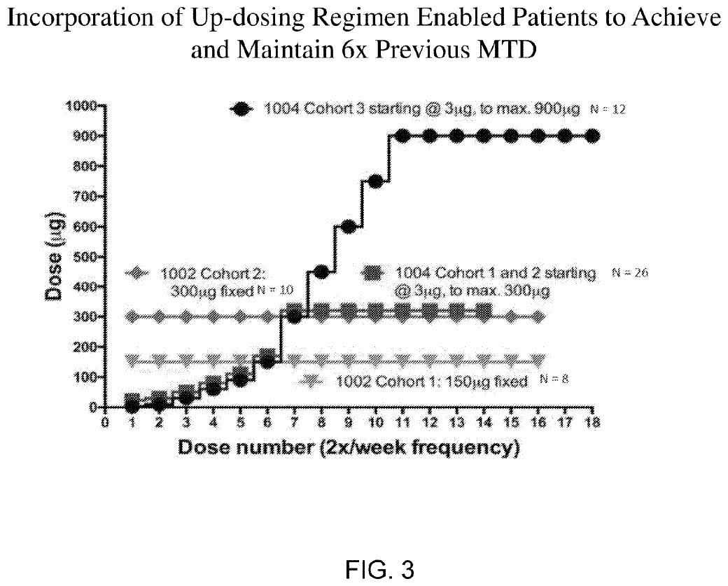

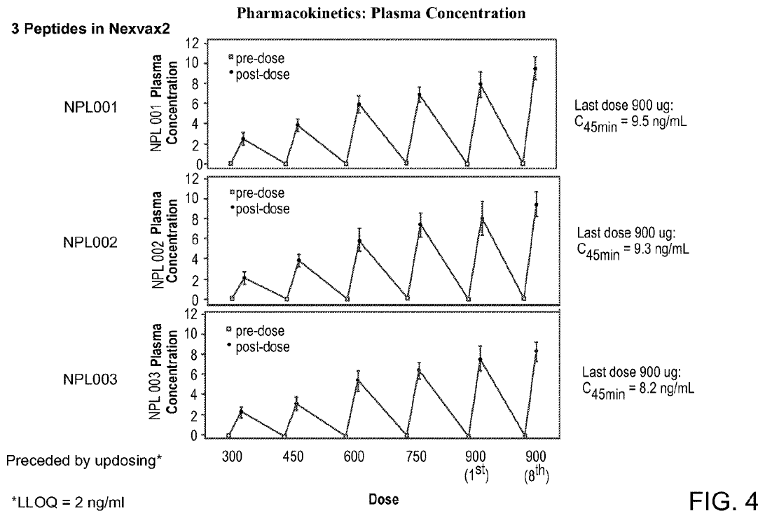

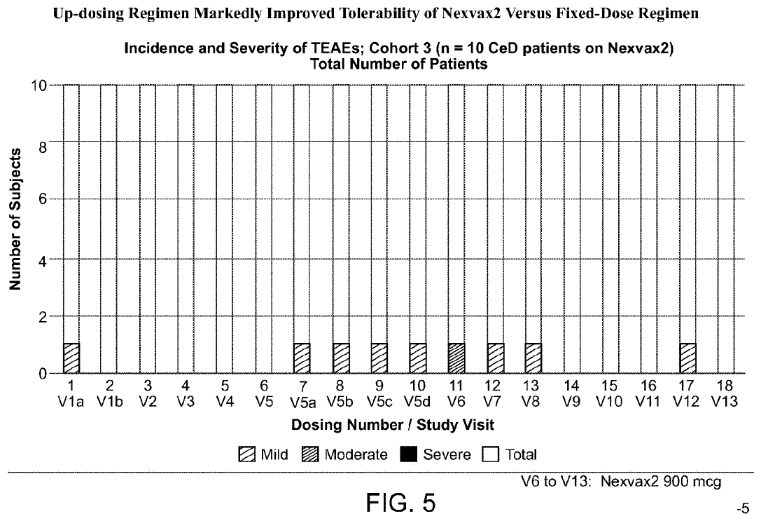

Thirty eight subjects (mean age 42 yr) were randomized 8:3, 10:5, or 10:2 to peptide composition or placebo in Cohorts 1, 2 and 3, respectively. All up-dosed patients tolerated and completed dosing at 900 micrograms. (FIG. 3). Both 300 microgram and 900 microgram doses were well-tolerated, including during the up-dosing titration. Treatment-related adverse events were mild and self-limiting. Pharmacokinetics of gluten peptides in plasma is shown in FIG. 4. The up-dosing regimen markedly improved the tolerability of peptide composition versus fixed-dose regimen (FIG. 5).

The second subject enrolled in Cohort 1 withdrew after 2 doses (30 micrograms and 60 micrograms) with severe abdominal pain, which led to a reduction in starting dose (3 micrograms). TEAEs with up-dosing from 3 micrograms and maintenance at 300 micrograms or 900 micrograms were mild or moderate apart from 1 subject in Cohort 2 who experienced a severe headache. Subjects who received placebo (n=9) had TEAEs similar to peptide composition treated subjects whose dosing started at 3 micrograms in Cohorts 1 (n=6) and 3 (n=10). Weekly mean GSRS decreased significantly each week after Week 3 of peptide composition treatment compared to baseline in Cohort 3 (p<0.05, Wilcoxon paired rank sum test).

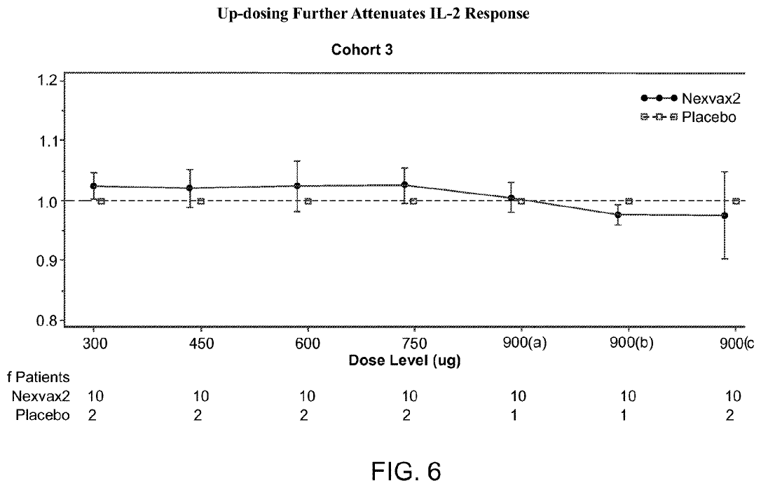

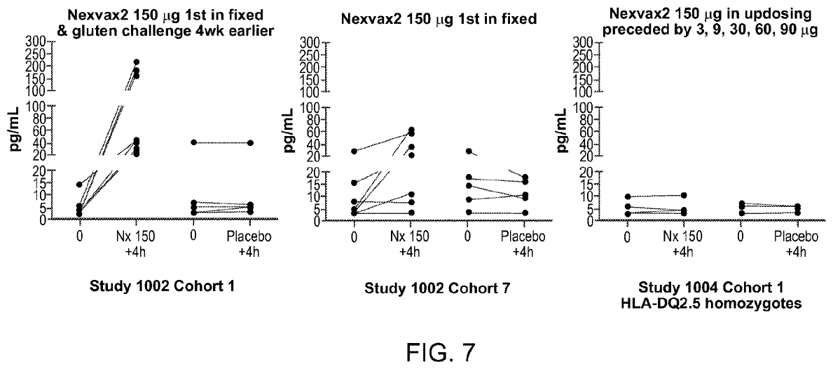

None of 38 cytokines were elevated in plasma at 4 h after ≥150 micrograms of peptide composition. No elevations in any cytokines or chemokines (e.g., IL-2, IL-8, MCP-1) at 4-hours post-dose following 150 microgram and subsequent dose levels were observed in any cohort. Up-dosing further attenuated the IL-2 response, as shown in FIG. 6. FIG. 7 is a series of graphs contrasting IL-2 release in plasma when comparing up-dosing (right panel) with fixed dosing (left and middle panel).

All peptide composition treated subjects in Cohort 3 had quantifiable, dose-dependent plasma levels of each of the peptides (˜9 ng/mL after 900 micrograms).

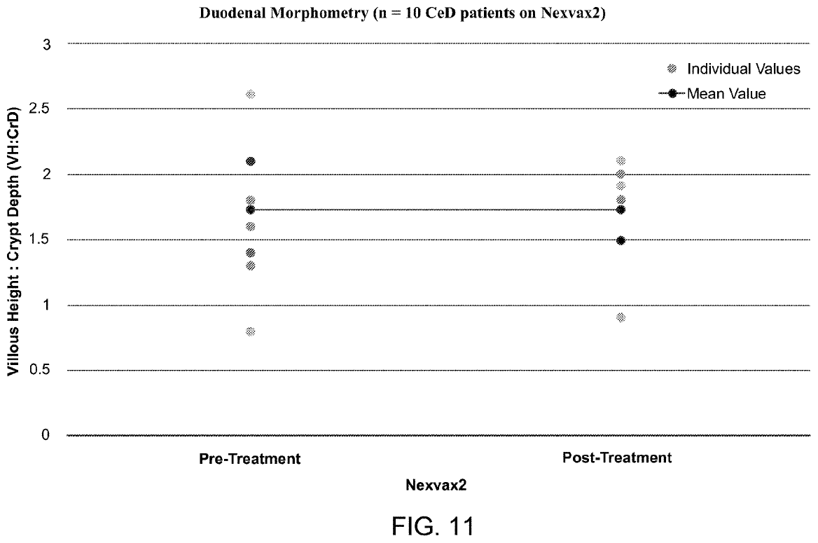

No overall change in duodenal histology compared to baseline was observed (FIG. 11). Mean (95% CI) duodenal VH:CrD was 1.7 (1.3-2.1) before and 1.7 (1.4-1.9) after treatment with peptide composition.

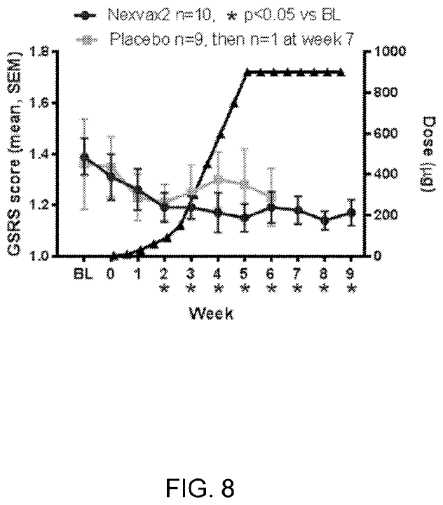

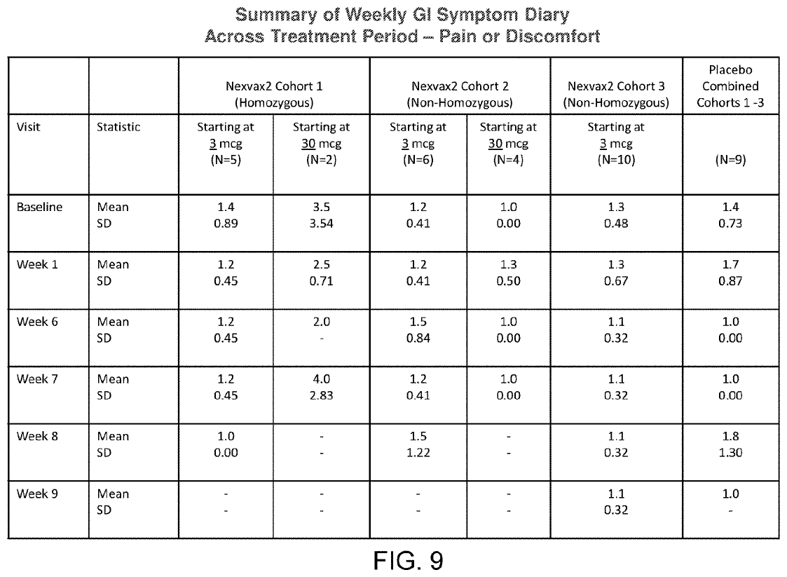

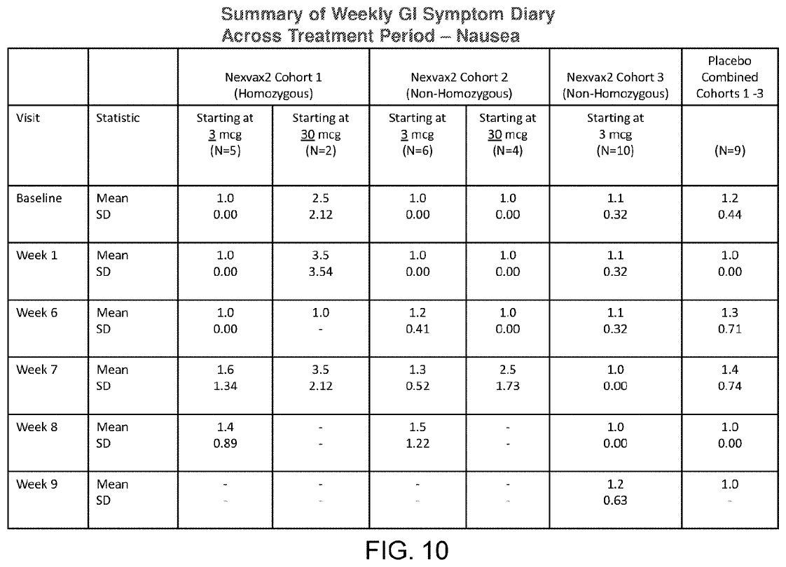

FIG. 8 shows that the treatment is associated with sustained reduction of symptoms per weekly GSRS (patient reported). FIG. 8 is a graph depicting Gastrointestinal Symptom Rating Scale (GSRS) score over time (lower numbers indicate lesser symptom severity). Overall symptom scores were measured at baseline and then weekly. There were 15 GI system domains. Placebo patients pooled all cohorts. Up-dosing began at 3 micrograms and the top dose was 900 micrograms. A significant reduction in symptoms compared to baseline was seen. No difference in symptoms between baseline and treatment period was seen in the placebo group. Tables summarizing the weekly GI symptom diary across treatment period related to pain or discomfort and the weekly GI symptom diary across treatment period related to nausea can be found respectively in FIGS. 9 and 10.

This example demonstrates that up-dosing enabled, among other things, achievement of a 900 microgram dose, which is 6 times higher versus a fixed-dose regimen. Up-dosing also enabled a well-tolerated regimen with a clean adverse events (AE) profile, which is significantly improved as compared to a fixed-dose regimen.

### Example 7. Epitope-Specific Immunotherapy Targeting CD4-Positive T Cells in Coeliac Disease: Evaluation of Escalating Dose Regimens of Nexvax in a Randomised, Double-Blind, Placebo-Controlled Phase 1 Study

Nexvax2® is a novel, peptide-based, epitope-specific immunotherapy intended to be administered by regular injections at dose levels that increase the threshold for clinical reactivity to natural exposure to gluten and ultimately restore tolerance to gluten in patients with coeliac disease. Coeliac disease patients administered fixed intradermal doses of Nexvax2 become unresponsive to the HLA-DQ2.5-restricted gluten epitopes in Nexvax2, but gastrointestinal symptoms and cytokine release mimicking gluten exposure that accompany the first dose limit the maximum tolerated dose to 150 μg. Our aim was to test whether stepwise dose escalation attenuated the first dose effect of Nexvax2 in coeliac disease patients.

### Methods

We conducted a randomised, double-blind, placebo-controlled trial at four community sites in Australia (3) and New Zealand (1) in HLA-DQ2.5 genotype positive adults with coeliac disease who were on a gluten-free diet. Participants were assigned to cohort 1 if they were HLA-DQ2.5 homozygotes; other participants were assigned to cohort 2, or to cohort 3 subsequent to completion of cohort 2. Manual central randomisation without blocking was used to assign treatment for each cohort. Initially. Nexvax2-treated participants in cohorts 1 and 2 received an intradermal dose of 30 μg (consisting of 10 μg of each constituent peptide), followed by 60 μg, 90 μg, 150 μg, and then eight doses of 300 μg over six weeks, but this was amended to include doses of 3 μg and 9 μg and extended over a total of seven weeks. Nexvax2-treated participants in cohort received doses of 3 μg, 9 μg, 30 μg, 60 μg, 90 μg, 150 μg, 300 μg, 450 μg, 600 μg, 750 μs, and then eight of 900 μg over nine weeks. The dose interval was 3 or 4 days. Participants, care providers, data managers, sponsor personnel, and study site personnel were blinded to treatment assignment. The primary outcome was the number of adverse events and percentage of participants with adverse events during the treatment period.

### Findings

From the 73 participants who we screened, 24 did not meet eligibility criteria, and 36 were ultimately randomised and received study drug. For cohort 1, seven participants received Nexvax2 (two with the starting dose of 30 μg and then five at 3 μg) and three received placebo. For cohort 2, 10 participants received Nexvax2 (four with starting dose of 30 μg and then six at 3 μg) and four received placebo. For cohort 3, 10 participants received Nexvax2 and two received placebo. All 36 participants were included in safety and immune analyses, and 33 participants completed treatment and follow-up; in cohort 3, 11 participants were assessed and included in pharmacokinetics and duodenal histology analyses. Whereas the maximum dose of Nexvax2 had previously been limited by adverse events and cytokine release, no such effect was observed when dosing escalated from 3 μg up to 300 μg in HLA-DQ2.5 homozygotes or to 900 μg in HLA-DQ2.5 non-homozygotes. Adverse events with Nexvax2 treatment were less common in cohorts 1 and 2 with the starting dose of 3 μg (72 for 11 participants) than with the starting dose of 30 μg (91 for six participants). Adverse events during the treatment period in placebo-treated participants (46 for nine participants) were similar to those in Nexvax2-treated participants when the starting dose was 3 μg in cohort 1 (16 for five participants), cohort 2 (56 for six participants), and cohort 3 (44 for 10 participants). Two participants in cohort 2 and one in cohort 3 who received Nexvax2 starting at 3 μg did not report any adverse event, while the other 33 participants experienced at least one adverse event. One participant, who was in cohort 1, withdrew from the study due to adverse events, which included abdominal pain graded moderate or severe and associated with nausea after receiving the starting dose of 30 μg and one 60 μg dose. The most common treatment-emergent adverse events in the Nexvax2 participants were headache (52%), diarrhoea (48%), nausea (37%), abdominal pain (26%), and abdominal discomfort (19%). Nexvax2 treatment was associated with trends towards improved duodenal histology. Plasma concentrations of Nexvax2 peptides were dose-dependent. It was shown that antigenic peptides recognized by CD4-positive T cells in an autoimmune disease can be safely administered at high maintenance dose levels without immune activation if preceded by gradual dose escalation. Whereas the maximum dose of Nexvax2 had previously been limited to 150 μg by adverse events and cytokine release, no such effect was observed when dosing escalated from 3 μg up to 300 μg in participants with coeliac disease who were HLA-DQ2-5 homozygotes or to 900 μg in those who were HLA-DQ2.5 non-homozygotes. There was no evidence of immune activation or duodenal injury in response to Nexvax2 treatment, despite systemic exposure to Nexvax2 peptides.

Clinical and immunological reactivity to systemically administered antigenic gluten peptides are attenuated by recent exposure to lower dose levels of the same peptides. Unresponsiveness to high levels of systemic exposure to antigenic gluten peptides can be achieved in patients with coeliac disease following dose escalation.

### INTRODUCTION

“Immune tolerance” has been defined as “a state of indifference or non-reactivity towards a substance that would normally be expected to excite an immunological response”.¹ In patients with coeliac disease, immunological tolerance to dietary gluten is replaced by a T cell-mediated hypersensitivity reaction that results in small intestinal injury and digestive symptoms.²

Quarantining the immune system with a life-long, strict, gluten-free diet is currently the mainstay of management for coeliac disease.³ Gluten-free diet for six months or more usually results in normalisation of serum antibodies specific for gluten-derived peptides and autoantibodies specific for transglutaminase, but signs of ongoing intestinal injury persist in many patients.³ Recurrent digestive symptoms on gluten-free diet are common, and the risk of acute symptoms that can follow within hours of accidental gluten exposure is ever present.⁴ The shortcomings of a gluten-free diet highlight a substantial unmet need that is being addressed by clinical development of agents that may enhance the effectiveness of dietary therapy.⁵ However, overcoming the gluten-specific adaptive immune response and ultimately restoring immune tolerance without global immunosuppression is the long-term goal of pharmacotherapy for autoimmune diseases, including coeliac disease.⁶ In this study, an objective was to determine the safety and tolerability of Nexvax2 administered at maintenance dose levels of 300 μg or 900 μg when preceded by dose titrations in patients with coeliac disease on a gluten-free diet.

### Methods

### Study Design

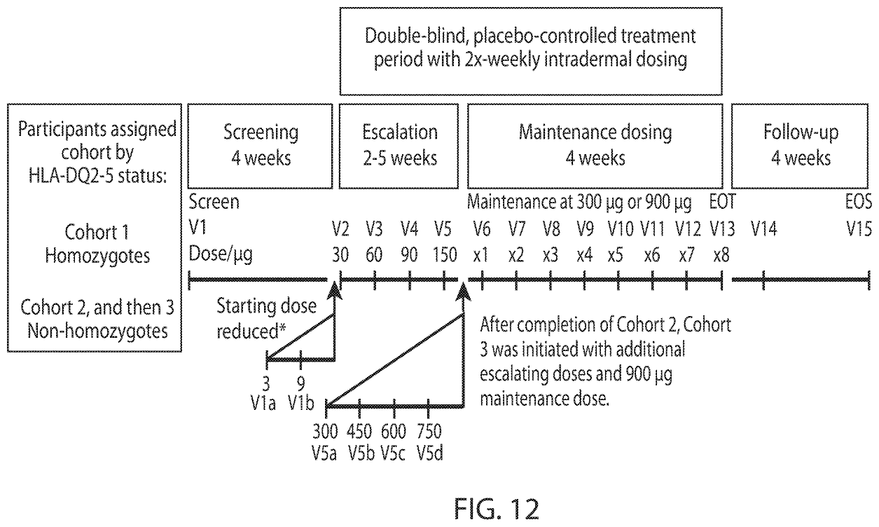

Nexvax2 was administered by stepwise dose escalation followed by a high maintenance dose in this randomised, double-blind, placebo-controlled phase 1 study. The study design is shown in FIG. 12. This study was conducted at four community sites in Australia (3) and New Zealand (1).

### Participants

Participants were required to be between 18 and 70 years old, have a coeliac disease diagnosis on the basis of intestinal histology demonstrating villous atrophy, and possess both alleles encoding HLA-DQ2.5. At the screening visit, participants were excluded if they had not maintained a gluten-free diet for at least one year, had elevated serology for both transglutaminasc 2 IgA and deamidated gliadin peptide IgG, or had a score of more than 12 on the Coeliac Dietary Adherence Test (CDAT) consistent with reduced adherence to gluten-free diet.¹⁷ Eligible participants were enrolled in cohort 1 if they had HLA-DQA1*05 and HLA-DQB1*02 alleles and no other HLA-DQA or HLA-DQB alleles (HLA-DQ2.5 “homozygotes”), whereas other eligible participants (HLA-DQ2.5 “non-homozygotes”) were enrolled in cohort 2 or, subsequently, in cohort 3.

### Randomisation and Masking

Manual central randomisation without blocking was used for each cohort. The randomisation schedule was generated with SAS v9·3 (SAS Institute Inc., Cary, N.C., USA) and remained sequestered until database lock. Participants were randomised to receive Nexvax2 or placebo 8:3 in cohorts 1 and 2, and 10:2 in cohort 3. Replacements were allowed, and they received identical treatment as the participant being replaced. Study drug were shipped to the study site in double-blind treatment kits according to the randomisation assignment. Study site personnel and sponsor received only the unique randomisation number, the date of randomisation, and the treatment kit assignment. The appearance of vials, the drug product, the volume injected, and the number of injections for Nexvax2 and placebo treatments were identical within each cohort. Study participants, care providers, data managers, sponsor personnel, and study site personnel remained blinded to study treatment assignment until database lock for each cohort.

### Procedures

At the screening visit, participant eligibility was determined by assessing the level of compliance to a gluten-free diet and the results of a physical examination, electrocardiogram, and blood tests, including coeliac disease serology and HLA-DQA and HLA-DQB genotype. Digestive symptoms over the previous week were assessed at the screening visit and weekly until after the treatment period using the Gastrointestinal Symptom Rating Scale (GSRS).¹⁸ Participants in cohort 3 also had an upper gastrointestinal endoscopy to assess second part duodenal histology. Within four weeks of the screening visit, eligible participants were randomised and began the treatment period.

Participants received study drug administered by staff at the study site. Intradermal injections were administered to the abdomen at the level of the waist alternating between the right and left of the body twice per week (3- or 4-day intervals) for up to nine weeks according to the regimens shown in FIG. 12. The treatment period was divided between an up-dosing phase and a four-week maintenance phase when eight doses of Nexvax2 were administered at 300 μg in cohorts 1 and 2, or at 900 μg in cohort 3. The up-dosing regimen for cohorts 1 and 2 was initially 30, 60, 90, and 150 μg, but was subsequently amended to 3, 9, 30, 60, 90, and 150 μg. The up-dosing regimen for cohort 3 was 3, 9, 30, 60, 90, 150, 300, 450, 600, and 750 μg. Dose levels below 300 μg could be administered only once, whereas dose levels from 450 to 750 μg could be administered up to a total of three times. Down-dosing to the next lowest dose was allowed if dose levels from 450 to 900 μg were poorly tolerated after three administrations. Safety assessments during the treatment period included vital signs, clinical pathology, and adverse event monitoring. Adverse events were recorded at each visit, which were graded by site staff according to Common Terminology Criteria for Adverse Events (CTCAE) v4.03.

Pharmacodynamics assessments included a 38plex assay to profile cytokine and chemokine concentrations in plasma before and up to 10 hours post-treatment at visits corresponding to administration of Nexvax2 at the previously determined maximum tolerated dose (150 μg) and at each of the higher dose levels. The percentage of leukocytes in whole blood that corresponded to T cells or helper, cytotoxic, regulatory, or activated (CCR6-positive) T cell subsets was estimated using epigenetic cell counting before and after dosing during the treatment period at times indicated. Pharmacokinetics of the three constituent peptides in Nexvax2 were assessed pre-treatment and 45 minutes post-treatment in cohort 3 at visits corresponding to dose levels 300 μg and above. Serum levels of anti-Nexvax2 antibodies were also assessed in cohort 3 at times shown. A four-week observational period followed the end of treatment visit. Participants in cohort 3 had an upper gastrointestinal endoscopy to assess second part duodenal histology within one week of completing the treatment period.

### Outcomes

All outcomes were centrally assessed. The pre-specified primary outcome was the number and percentage of adverse events during the treatment period. The following pre-specified secondary outcomes were also assessed: 1) weekly GSRS scores during the treatment period; 2) in cohort 3, pharmacokinetics of Nexvax2 at the first administration of 300, 450, 600, 750, and 900 μg doses and at the end of treatment; 3) in cohort 3, the effect of Nexvax2 at 900 μg on duodenal histology, as determined by the change in villous height to crypt depth ratio from baseline screening to end of treatment; and 4) relative change in the concentration of plasma cytokines and chemokines after sequential doses of Nexvax2.

### Statistical Analysis

A sample size of 34 participants was planned for this study, including randomisation of approximately 22 participants for cohorts 1 and 2 and randomisation of approximately 12 participants for cohort 3. The sample size was chosen pragmatically to permit assessment of safety and tolerability of Nexvax2 while limiting unnecessary exposure. The following study populations were used in the statistical analyses: the safety population included all participants who received a dose of Nexvax2 or placebo (analysed according to treatment actually received); the gastrointestinal symptom score population included all participants who received a dose of Nexvax2 or placebo and had at least one assessment of the GSRS after dosing (analysed according to treatment actually received); the pharmacokinetics population included all participants in cohort 3 who received at least 300 μg of Nexvax2.

Descriptive statistics was used to summarise demographic data and baseline participant characteristics. Adverse events were presented as numbers and percentage of participants.

Pharmacokinetics of Nexvax2 peptides was summarised by dose level and presented as mean (95% CI) plasma concentrations; correlation coefficients were used to compare the plasma concentrations of the Nexvax2 peptides. The paired, non-parametric Wilcoxon's signed-rank test was used to compare GSRS scores over time and between treatment groups and to compare the change in villous height to crypt depth ratio between treatment groups. Cytokine data were presented as median fold change from pre-treatment levels. Data from cohorts 1 and 2 were analysed separately according to the Nexvax2 starting dose levels of 3 μg or 30 μg. Data were collected by investigators and managed by CPR Pharma Services, and statistical analyses were performed by PROMETRIKA, LLC (Cambridge, Mass., USA). SAS v9.4 and Prism v6 (GraphPad Software, Inc., La Jolla, Calif., USA) were used for statistical analyses.

### Results

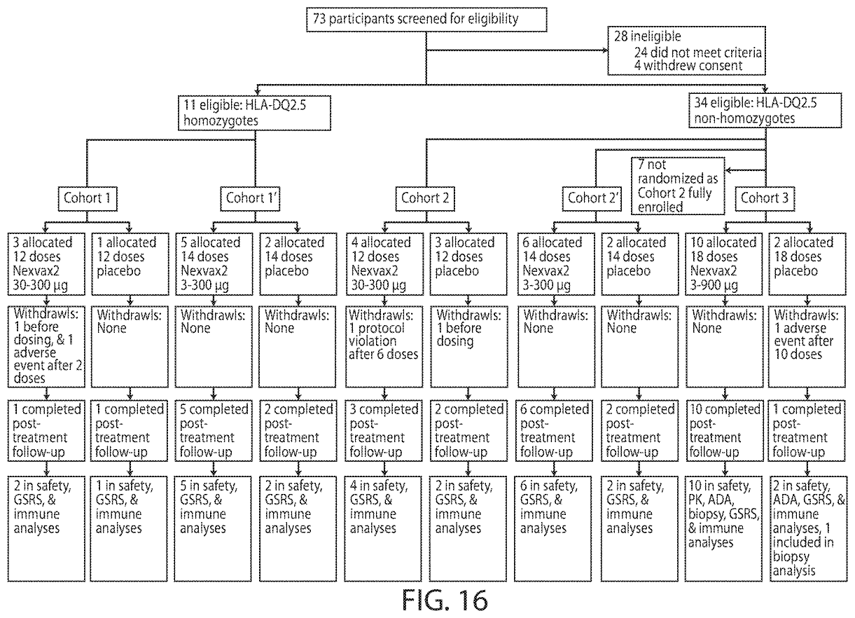

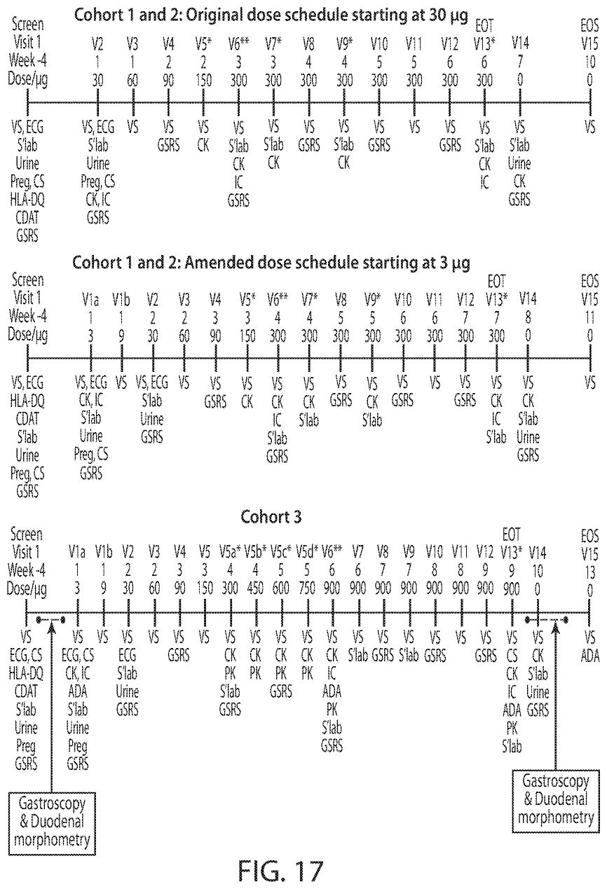

Volunteers were screened for eligibility of whom 45 were eligible and 36 ultimately received investigational product (FIG. 16). Recruitment was slower for cohort 1 because HLA-DQ2.5 homozygotes constitute only about 20% of patients diagnosed with coeliac disease.¹⁹ By a certain time point, three HLA-DQ2.5 homozygotes had been recruited into cohort 1 (two randomised to Nexvax2 and one randomised to placebo), while six non-homozygotes had been recruited to cohort 2 (four randomised to Nexvax2 and two randomised to placebo). For these participants, the Nexvax2 starting dose was 30 μg and their assigned treatment included a total of 12 doses with four in the up-dosing phase. For participants enrolled after that time, the dosing regimen was amended with the aim of improving tolerability of the starting dose. For the seven subsequent participants in cohort 1 (five randomised to Nexvax2 and two randomised to placebo) and eight participants in cohort 2 (six randomised to Nexvax2 and two randomised to placebo), the Nexvax2 starting dose was 3 μg and their assigned treatment included a total of 14 doses with six in the up-dosing phase. By an even later point in time, a total of 15 eligible HLA-DQ2.5 non-homozygotes were enrolled into cohort 2 (10 randomised to Nexvax2 and five to placebo, with one participant randomised to placebo withdrawing prior to dosing). Ten months later, all 11 eligible volunteers who were HLA-DQ2.5 homozygotes were entered into cohort 1 with eight randomised to Nexvax2 and three to placebo, though one participant randomised to Nexvax2 withdrew before dosing.

After completion of cohort 2 and before opening enrolment of cohort 3, seven eligible HLA-DQ2.5 non-homozygotes were screened but not randomised. After interim analysis of findings from cohort 2, all 12 eligible HLA-DQ2.5 non-homozygotes screened for a time period entered into cohort 3, with 10 randomised to Nexvax2 and two randomised to placebo.

Six participants who commenced treatment did not complete the assigned number of doses. For two participants (one receiving Nexvax2 and one placebo) this was due to early withdrawal due to adverse events, and for one participant receiving Nexvax2 discontinuation was due to a protocol violation (gluten exposure). In addition, two participants missed one or two consecutive maintenance doses of 300 μg or 900 μg, respectively, and one participant repeated the 600 μg dose during escalation.

One of two participants enrolled in the initial group in cohort 1 who received Nexvax2 starting at 30 μg withdrew consent after the second dose in the up-dosing phase following adverse events considered to be study drug related. After the initial 30 μg Nexvax2 starting dose, this participant had onset of upper abdominal pain graded severe, which lasted for one hour and was associated with mild nausea. Three days later, after the second dose of Nexvax2 (60 μg), there was onset of abdominal pain and nausea both graded moderate, which were accompanied by arthralgia, mental ‘fogginess’, and perspiring, each graded mild. The protocol was revised following this participant's withdrawal so that the up-dosing phase began with Nexvax2 doses of 3 μs and 9 μg. One participant in cohort 2 received six doses of Nexvax2 including two doses at 300 μg before being discontinued from the study because of a protocol violation of unintended non-adherence to gluten-free diet. Approximately 7 hours after the fifth dose, food containing gluten was consumed inadvertently, which was followed between 2 and 3 hours later by abdominal pain graded moderate and fatigue, nausea, vomiting, and diarrhoea, each graded mild. One participant in cohort 3 who received 10 doses of placebo withdrew from the study due to an intervertebral disc protrusion graded severe and unrelated to study drug. One replacement participant was enrolled in cohort 1 and randomised to Nexvax2. Two replacement participants were enrolled in cohort 2 (one randomised to placebo and one randomised to Nexvax2). Altogether, 33 participants completed treatment out of 36 participants who received at least one dose of Nexvax2 or placebo; all 36 participants were included in the primary outcome safety population analyses.

Median age of the 36 participants who received at least one dose of Nexvax2 or placebo was 41 years (IQR 32.0 to 52.8), and 25 (69%) were women (table 1). Median age at coeliac disease diagnosis was 33.5 years (IQR 27.5 to 41.0); median time since diagnosis was 6.5 years (IQR 3.8 to 12.3); and median time on a gluten-free diet was 5.5 years (IQR 3.0 to 11.5). Participants in each cohort of the Nexvax2 (n=27) and placebo (n=9) groups displayed similar demographics, baseline coeliac disease-specific serology, and gene dose for the alleles that code HLA-DQ2.5 (table 1).

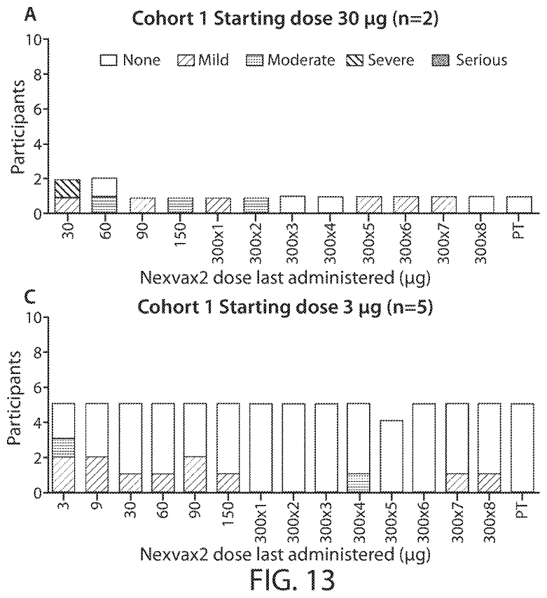

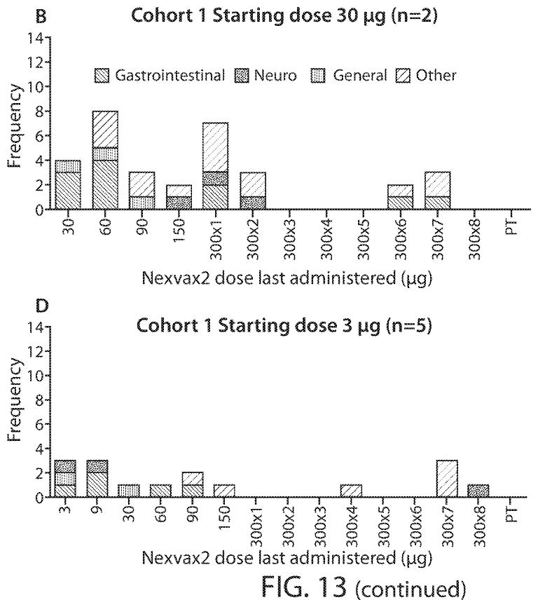

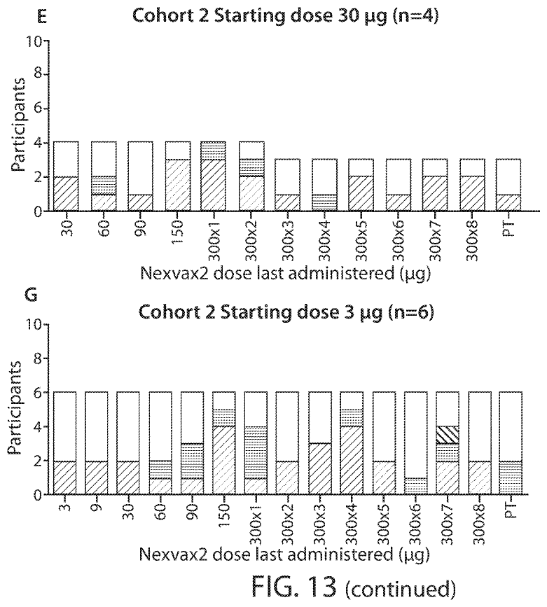

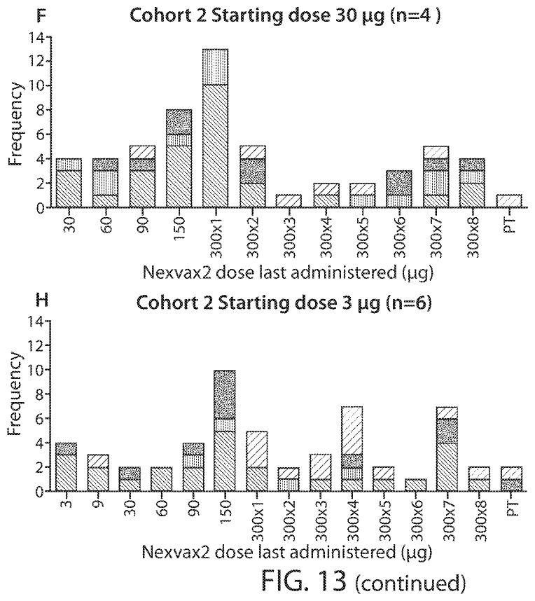

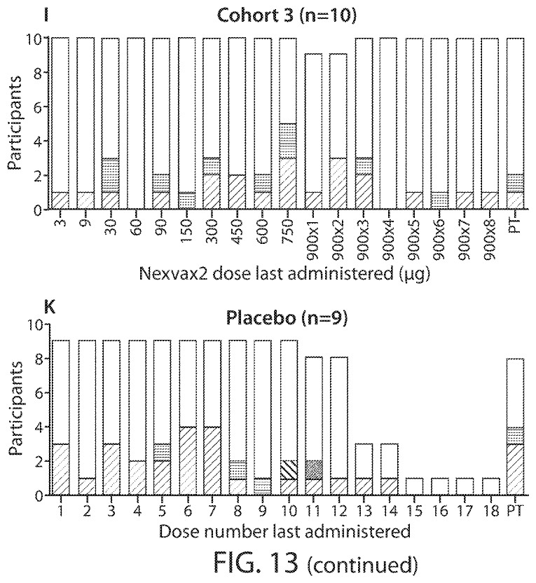

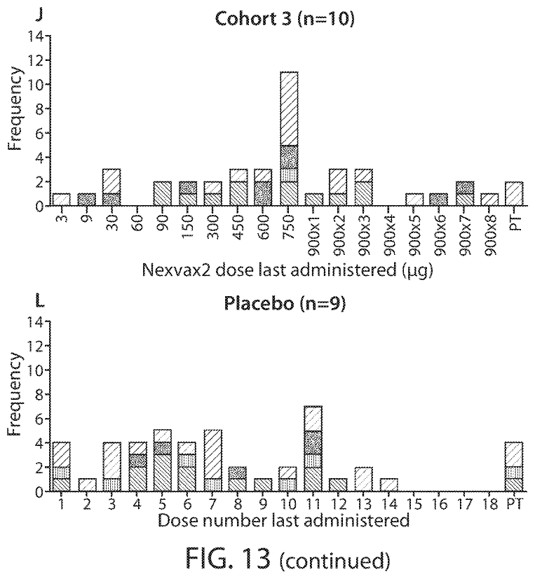

The total number of treatment-emergent adverse events in the 27 participants who received Nexvax2 was 207 compared with 46 in nine participants who received placebo (table 2). Overall, 24 (89%) of the 27 participants receiving Nexvax2 experienced at least one treatment-emergent adverse event compared with nine (100%) of nine participants who received placebo (table 3). There was no particular dose level consistently associated with increased frequency of adverse events (FIG. 13). In the Nexvax2-treated participants, 136 (66%) of the 207 treatment-emergent adverse events were considered related to the study drug compared with 25 (54%) of the 46 treatment-emergent adverse events in placebo-treated participants. There were two serious adverse events (somnolence and intervertebral disc protrusion), both of which affected placebo-treated participants. Participant vital signs were measured before and after dosing; there were no remarkable findings in the vital signs of participants in the Nexvax2 or placebo groups, and treatment with Nexvax2 did not result in any treatment-related changes in ECG readings or physical examination.

In cohort 1, two participants had shorter duration up-dosing, and the higher Nexvax2 starting dose of 30 μg accounted for 34 (68%) of all adverse events reported for Nexvax2-treated participants in this cohort (FIG. 13 and table 2), even though one of these two participants discontinued after only 2 doses. The four (40%) participants in cohort 2 who had shorter duration up-dosing and the higher Nexvax2 starting dose of 30 μg, including one participant who had an inadvertent gluten exposure, contributed 57 (50%) of the treatment-emergent adverse events in cohort 2 (table 2). Altogether there were 50 treatment-emergent adverse events in the seven participants who received Nexvax2 in cohort 1, 113 in the 10 participants who received Nexvax2 in cohort 2, 44 in the 10 participants who received Nexvax2 in cohort 3, and 46 in the nine participants who received placebo (table 3). Treatment-emergent adverse events affecting the gastrointestinal system accounted for 83 (40%) of the 207 treatment-emergent adverse events in the 27 participants who received Nexvax2 compared with 14 (30%) of 46 treatment-emergent adverse events in the nine participants who received placebo (table 3). Altogether there were 16 treatment-emergent gastrointestinal adverse events in the seven participants who received Nexvax2 in cohort 1, 54 in the 10 participants who received Nexvax2 in cohort 2, and 13 in the 10 participants who received Nexvax2 in cohort 3. Five (71%) of seven participants who received Nexvax2 in cohort 1 reported at least one episode of a treatment-emergent gastrointestinal adverse event, as did 10 (100%) of 10 who received Nexvax2 in cohort 2, seven (70%) of 10 who received Nexvax2 in cohort 3, and six (67%) of nine who received placebo. Treatment-emergent adverse events affecting the nervous system were second most common overall and accounted for 34 (16%) of the 207 treatment-emergent adverse events in the 27 participants who received Nexvax2 compared with 6 (13%) of 46 treatment-emergent adverse events in the nine participants who received placebo.

The most common individual treatment-emergent adverse events reported for Nexvax2-treated participants were headache in 14 (52%), diarrhoea in 13 (48%), nausea in 10 (37%), abdominal pain in seven (26%), abdominal discomfort in five (19%), and fatigue in five (19%) (table 3). In the Nexvax2 group, the only instance of treatment-emergent vomiting was in one participant in cohort 2 who inadvertently consumed gluten after the first maintenance dose. Adverse events classified as injection site reactions were all graded mild and included two (22%) of nine participants who received placebo and nine (33%) of 27 participants who received Nexvax2. Among those participants who experienced injection site reactions, there were five (24%) of 21 Nexvax2-treated participants with a starting dose at 3 μg (each experienced one injection site reaction) and four (67%) of six with a starting dose at 30 μg, who accounted for 12 (71%) of the 17 injection site reaction adverse events in Nexvax2-treated participants.

For the six participants in cohorts 1 and 2 whose Nexvax2 starting dose was 30 pig, on average, four (67%) experienced adverse events after each of the first five Nexvax2 administrations concluding with the first 300 μg maintenance dose, with 31 (48%) out the total of 65 adverse events during this phase affecting the gastrointestinal system (FIG. 13). For the four Nexvax2-treated participants in cohorts 1 and 2 who received more than two 300 μg maintenance doses and whose starting dose was 30 μg, on average, two (50%) experienced adverse events after each of the last seven 300 μg maintenance doses.

Overall, in Nexvax2-treated participants whose starting dose was 3 μg, there was no specific dose level or dose number that was poorly tolerated (FIG. 13) or caused discontinuation; thus, no maximum tolerated dose was determined. There was one instance during the up-dosing phase when the same dose was repeated because of an adverse event; one participant in cohort 3 experienced arthralgia graded mild after receiving 600 μg of Nexvax2; this adverse event did not recur with repeat or higher doses. For the 21 participants in cohorts 1, 2, and 3 whose Nexvax2 starting dose was 3 μg, six (29%) experienced adverse events after each of the first seven Nexvax2 administrations up to 300 μg, with 17 (43%) out the total of 40 adverse events during this phase affecting the gastrointestinal system (FIG. 13). Adverse events following subsequent doses of Nexvax2 were similar to that observed in the placebo group. For the nine participants in cohorts 1, 2, and 3 who received placebo, on average, three (33%) experienced adverse events after each of the first seven placebo administrations with eight (28%) out the total of 29 adverse events during this phase affecting the gastrointestinal system (FIG. 13). For the 11 participants in cohorts 1 and 2 whose starting dose was 3 μg, on average, three (27%) experienced adverse events after each of the last seven 300 μs doses. For the 10 participants in cohort 3, on average, three (30%) experienced adverse events after each of the four Nexvax2 doses from 450 μg up to 900 μg; on average, one (10%) experienced adverse events after each of the subsequent seven 900 μg maintenance doses.

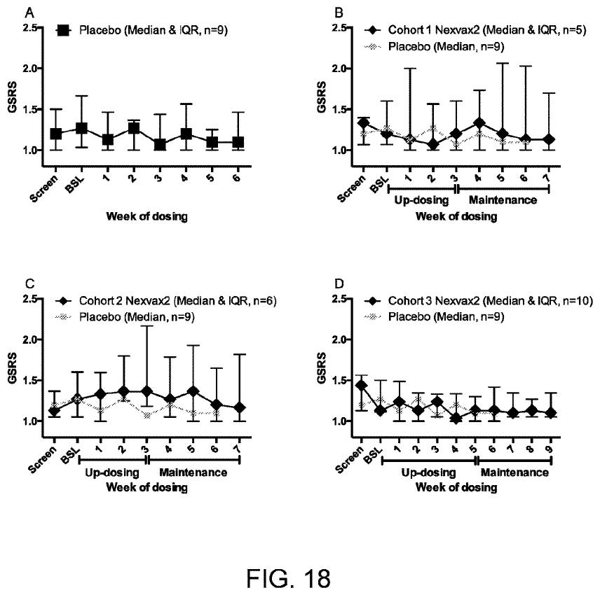

The average GSRS score was used to measure participant's digestive symptoms over the previous week (FIG. 18). For the nine participants who received placebo, three had lower average GSRS scores after six weeks of treatment than at baseline; of the remaining participants, three had the same scores and three had higher scores, resulting in a median difference between average GSRS scores between baseline and six weeks of zero (IQR −0.27 to 0.05). For the 21 participants who had a Nexvax2 starling dose of 3 μs and completed seven weeks of treatment in cohorts 1 and 2 or nine weeks of treatment in cohort 3, the average GSRS scores were lower at the end of treatment than at baseline in 13, the same in three, and higher in five participants. In cohort 3, participants who received Nexvax2 showed the highest median change in GSRS scores between baseline and end of treatment (−0.13, IQR −0.18 to −0.02), compared with cohort 1 (−0.07. IQR −0.13 to 0.06) and cohort 2 (−0.04, IQR −0.12 to 0).

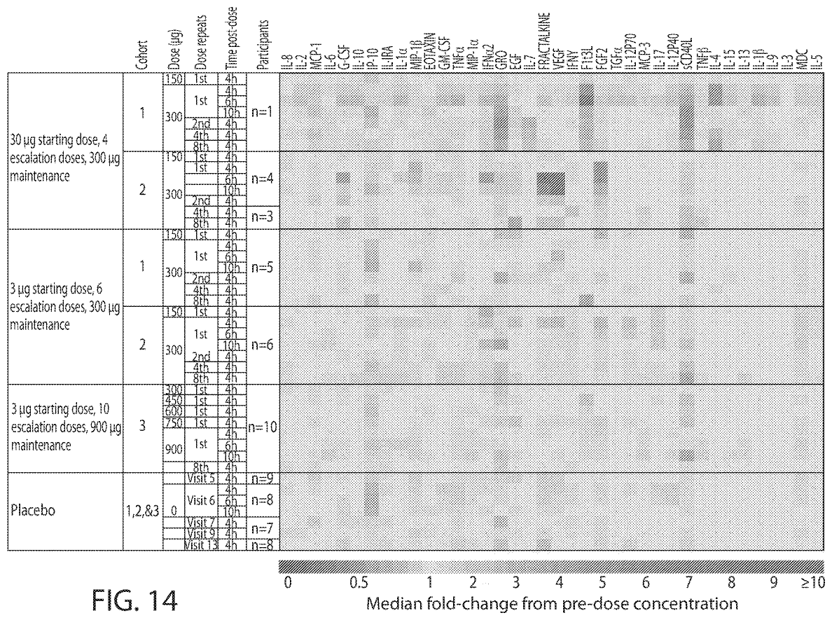

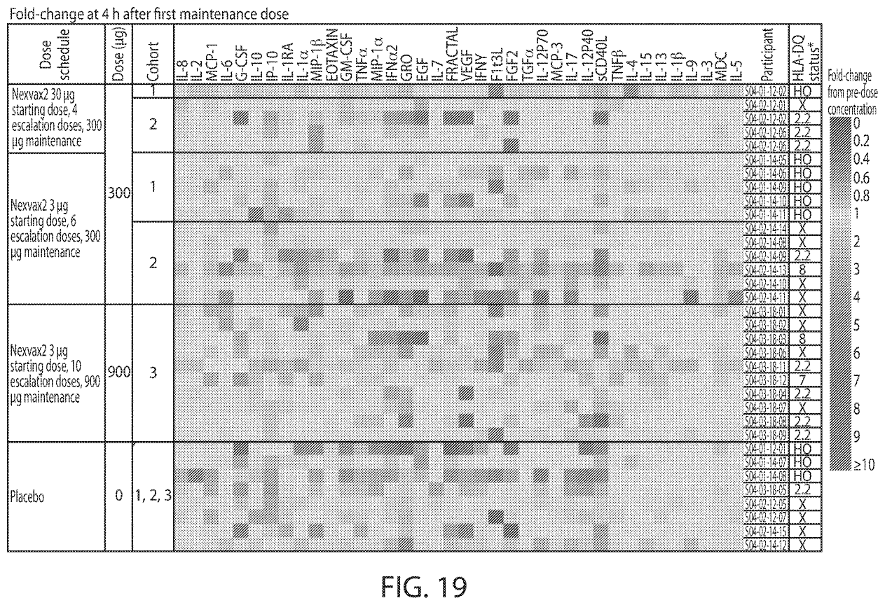

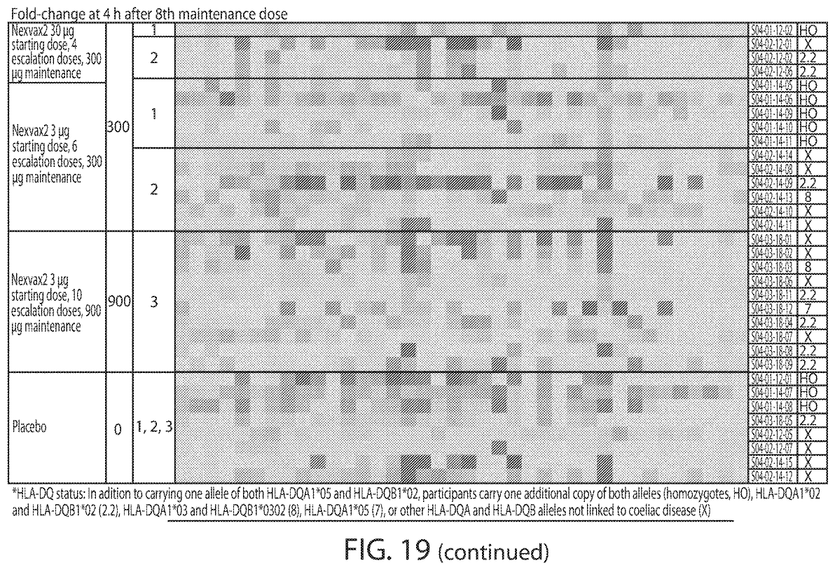

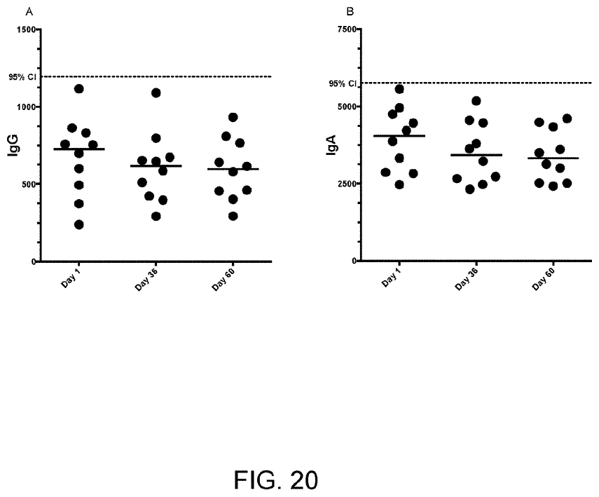

Relative change in the concentration of plasma cytokines and chemokines after sequential doses of Nexvax2 was a secondary endpoint. Acute elevations in plasma IL-8, IL-2, MCP-1, IL-6, IL-10, and IP-10 after the first 150 μs dose of Nexvax2 in fixed dose regimen studies were observed. In participants who had a Nexvax2 starting dose of 3 μs, the first administrations of Nexvax2 at 150 μg, 300 μs, or 900 μg were not associated with acute elevations in plasma cytokines or chemokines (FIG. 14 and FIG. 19).

Changes in duodenal histology were assessed in 10 participants following up-dosing to and maintenance of Nexvax2 at 900 μg, and in one placebo-treated participant over the nine-week treatment period. The number of participants was insufficient to infer any beneficial effect of Nexvax2, but overall, for Nexvax2-treated participants, duodenal morphology assessments were stable or showed trends towards improvement. Median villous height to crypt depth ratio before treatment was 1.62 (IQR 1.33 to 1.98) and post-treatment 1.78 (IQR 1.55 to 1.88; p=0.9688, Wilcoxon's signed-rank test); median villus height before treatment was 300.0 μm (IQR 275.4 to 338.4) compared with post-treatment 343.7 μm (IQR 302.3 to 357.3; p=0.156), and the median value for the sum of paired villus height and crypt depth measurements before treatment was 484.3 μm (IQR 473.8 to 528.2) compared with post-treatment 540.3 μm (IQR 528.4 to 569.9; p=0.065). Crypt depth, and the frequency of intraepithelial lymphocytes were stable in Nexvax2-treated participants.

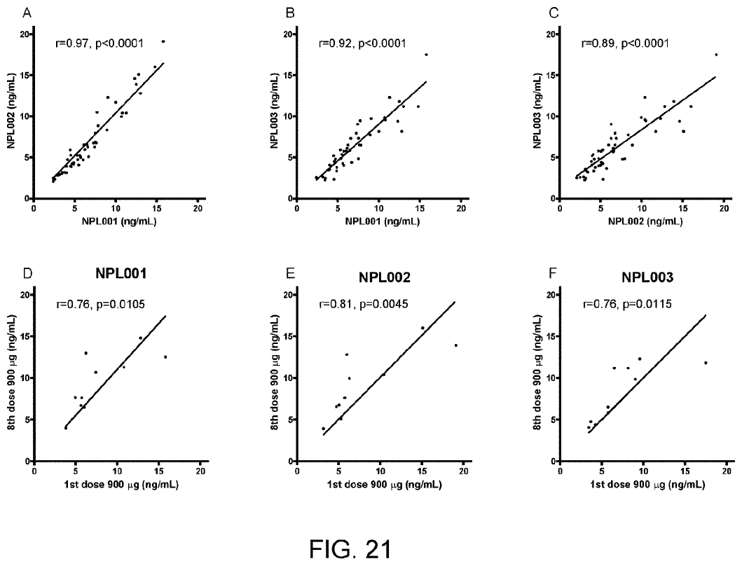

For participants in cohort 3, serum assessments of transglutaminase 2-specific IgA and deamidated gliadin peptide-specific IgG were repeated at the end of treatment. These assessments were in the normal range except in two participants who had elevated deamidated gliadin peptide-specific IgG, which in one case was not elevated before treatment but was not accompanied by change in quantitative histology (1.8 before and after treatment). In addition, for participants in cohort 3, serum levels of IgG and IgA specific for Nexvax2 were assessed. Participants in cohort 3 who received Nexvax2 had serum levels of IgG and IgA specific for Nexvax2 that were below the 95% cut off levels established with sera from unaffected donors (FIG. 21). Median levels of IgG and IgA specific for Nexvax2 were stable in cohort 3 over the 60-day treatment period.

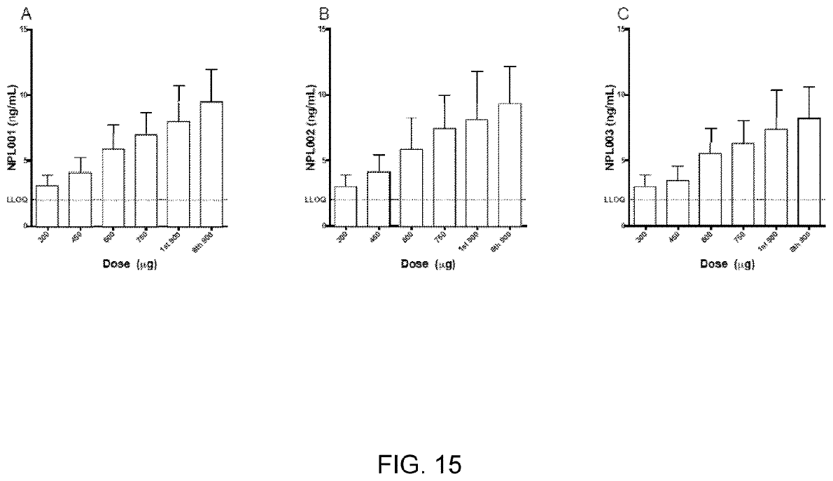

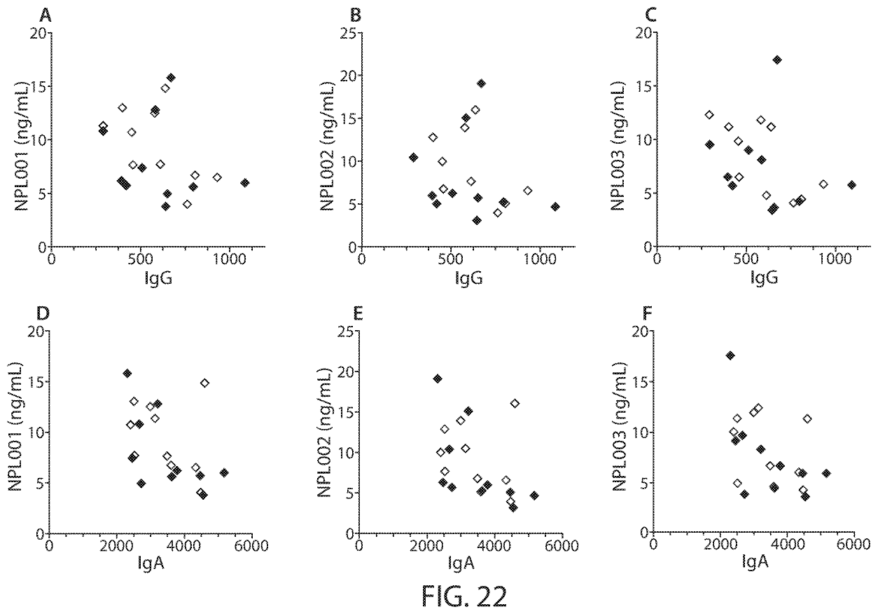

Because in previous phase 1 studies Nexvax2 peptides were detected in plasma from 10 minutes to 2 hours after administration of 300 μg of Nexvax2, albeit at concentrations below levels of quantitation, 12 we assessed the point plasma concentrations of Nexvax2 peptides in cohort 3 participants. An improved pharmacokinetics assay capable of measuring concentrations as low as 2 ng/mL for each peptide was used to assess plasma collected pre-treatment and 45 minutes post-treatment. In almost all participants, plasma concentrations of NPL001, NPL002, and NPL003 were above the limit of quantification 45 minutes after treatment at levels above 300 μg (FIG. 15). The three Nexvax2 peptides were not detected pre-treatment, and at 45 minutes post-treatment, displayed similar plasma concentrations that were consistent with dose-proportional kinetics. In addition, the 45-minute post-treatment concentrations of each Nexvax2 peptide correlated significantly with one another (FIG. 21, panels A-C) and were stable and correlated significantly between the first and last 900 μg doses (FIG. 21, panels D-F). No significant correlations were found between serum Nexvax2-specific IgG and IgA concentrations and the concentrations of the three Nexvax2 peptides (FIG. 22).

The relative change in T cell frequencies in whole blood during the treatment period was an exploratory endpoint. Epigenetic cell counting demonstrated that the percentages of leukocytes defined as T cells, and the subsets of T cells that were defined as regulatory, helper, CCR6-positive, and cytotoxic were stable from the first to last day of the treatment period in participants treated with Nexvax2 or placebo. T cell subset frequencies were also stable from pre-treatment to 4 hours or 10 hours after the first maintenance dose and from pre-treatment to 4 hours after the last maintenance dose.

### DISCUSSION

This study provides the first clinical evidence supporting the effectiveness of up-dosing in reducing adverse effects and in enabling higher maintenance dose levels for epitope-specific immunotherapy in a T-cell mediated autoimmune disease. It was found that a stepwise, intradermal up-dosing from a low, well tolerated starting dose allowed Nexvax2 to be administered without any increase in adverse effects at a maintenance dose 300× higher than the starting dose that was also 6× higher than the previously determined maximum tolerated dose. The frequency and severity of adverse events appeared to be more strongly influenced by the starting dose of Nexvax2 (3 μs or 30 μg) than by the maximum dose administered (300 μg or 900 μg). Dose inflexions during up-dosing were tolerated without any particular dose level being associated with an excess of adverse events. It was found that the adverse event profile during up-dosing from 3 μg to 300 μg was similar in HLA-DQ2.5 homozygotes and non-homozygotes. HLA-DQ2.5 non-homozygotes also tolerated further up-dosing from 300 μg to the maintenance dose of 900 μg, although this was not tested in HLA-DQ2.5 homozygotes due to their slower rate of recruitment. Self-reported gastrointestinal symptom scores were similar for treatment with Nexvax2 and placebo.

HLA-DQ2.5 positive volunteers with coeliac disease participating in previous studies frequently experienced acute gastrointestinal symptoms after the first administration of Nexvax2 in regimens with fixed doses ranging from 60 μg to 300 μg. In these studies, elevated plasma levels of IL-2 (a cytokine released by activated T cells), IL-6, IL-10, and the chemokines IL-8, MCP-1, and IP-10 were observed between two and six hours after the first dose. In keeping with the milder adverse event profile in the present study, no cytokine signature was observed up to 10 hours post-treatment with Nexvax2 from 150 μg to 900 μg. Occasional, but inconsistent, alterations in plasma chemokines were observed in some Nexvax2-treated participants who commenced up-dosing at 30 μg, which included one participant who inadvertently consumed gluten after receiving the first 300 μg dose.

Although we have previously detected the constituent Nexvax2 peptides in plasma after intra-dermal administration of Nexvax2, their levels were below limits of quantitation.¹² In the present study, we show for the first time that a peptide-based immunotherapy administered by intradermal injection has rapid, dose-dependent, systemic bioavailability that would facilitate engagement of cognate T cells at distant sites, including the gut, within minutes of administration.

Thus, the pharmacokinetics of Nexvax2 is consistent with other intradermally administered peptides that show dose-dependent pharmacokinetics similar to subcutaneous administration. Plasma concentrations of each of the three Nexvax2 peptides were similar at 45 minutes post-treatment. No difference was found in Nexvax2 pharmacokinetics after the first and eighth maintenance dose at 900 μg, which was associated with no change in serum Nexvax2-specific IgG and IgA levels.

Duodenal morphology was a safety measure to assess whether repeated administrations of “high” doses of Nexvax2 could mimic the deleterious effects of gluten exposure. We found that two-times weekly up-dosing over five weeks and maintenance for four weeks with Nexvax2 at the highest dose of 900 μg was associated with trends towards improving duodenal histology: villus length, the sum of villus height and crypt depth, and the villous height to crypt depth ratio trended upwards, and crypt depth was stable. However, only one placebo-treated participant was available for comparison, precluding further interpretation of changes in duodenal histology.

Nexvax2 is the first epitope-specific therapy to have detailed dose optimization using clinical adverse event monitoring, target organ histology, relevant immunological biomarkers in fresh blood, and patient segmentation according to gene dose for the restriction element. Nexvax2 is a simple, peptide-based, adjuvant-free formulation. In previous studies, the immunomodulation caused by Nexvax2 appeared to be gluten-specific, and there were no changes in recall immune responses after treatment with Nexvax2.12 In the present study, we provide further evidence that Nexvax2 did not cause systemic alterations in the frequencies of T cell subsets, including regulatory T cells during or following treatment with Nexvax2.

Although one limitation of this study was the small cohort sizes, participant demographics in these cohorts was consistent with the general population that suffers from coeliac disease, which is primarily white, non-Hispanic women.²¹ Another limitation is the small number of placebo-treated participants. In addition, although we have drawn comparisons between Nexvax2 fixed dosing and up-dosing regimens, we did not examine fixed dosing regimens in this study, but have relied instead on historical controls from our previous phase 1 studies.

Patients with coeliac disease having no excess of adverse events and no increasing plasma cytokine levels after dosing with Nexvax2 at dose levels as high as 900 μg supports the potential use of Nexvax2 maintenance treatment to protect against the effects of dietary gluten exposure. Our recent findings in patients with coeliac disease on a gluten-free diet indicate that the plasma cytokine signature associated with bolus administration of Nexvax2 is qualitatively and temporally indistinguishable from that following ingestion of gluten.¹² Daily consumption of gluten is about 10 to 14 grams in Europe and the United States,^{23,24} which suggests that the Nexvax2 dose level of 900 μg is relevant to test the efficacy of Nexvax2. Collectively, these results support the safety and tolerability of up-dosing and have allowed higher maintenance doses of Nexvax2 to be tested.

### REFERENCES

1. Medawar P B. Immunological tolerance. Science 1961; 133: 303-6.
2. Sollid L M, Jabri B. Triggers and drivers of autoimmunity: lessons from coeliac disease. Nat Rev Immunol 2013; 13: 294-302.
3. Ludvigsson J F, Bai J C, Biagi F, et al. Diagnosis and management of adult coeliac disease: guidelines from the British Society of Gastroenterology. Gut 2014; 63: 1210-28.
4. See J A, Kaukinen K, Makharia G K, Gibson P R, Murray J A. Practical insights into gluten-free diets. Nat Rev Gastroenterol Hepatol 2015; 12: 580-91.
5. Kurada S, Yadav A, Leffler D A. Current and novel therapeutic strategies in celiac disease. Expert Rev Clin Pharmacol 2016; 9: 1211-23.
6. Sabatos-Peyton C A, Verhagen J, Wraith D C. Antigen-specific immunotherapy of autoimmune and allergic diseases. Curr Opin Immunol 2010; 22: 609-15.
7. Larche M, Wraith D C. Peptide-based therapeutic vaccines for allergic and autoimmune diseases. Nat Med 2005; 11: S69-76.
8. Anderson R P, Jabri B. Vaccine against autoimmune disease: antigen-specific immunotherapy. Curr Opin Immunol 2013; 25: 410-7.
9. Streeter H B, Rigden R, Martin K F, Scolding N J, Wraith D C. Preclinical development and first-in-human study of ATX-MS-1467 for immunotherapy of M S. Neurol Neuroimmunol Neuroinflamm 2015; 2:e93.
10. Alhadj Ali M, Liu Y F, Arif S, Tatovic D, et al. Metabolic and immune effects of immunotherapy with proinsulin peptide in human new-onset type 1 diabetes. Sci Transl Med 2017; 9. pii: eaaf7779.
11. Tye-Din J A, Stewart J A, Dromey J A, et al. Comprehensive, quantitative mapping of T cell epitopes in gluten in celiac disease. Sci Transl Med 2010; 2: 41ra51.
12. Goel G, King T, Daveson A J, et al. Epitope-specific immunotherapy targeting CD4-positive T cells in coeliac disease: two randomised, double-blind, placebo-controlled phase 1 studies. Lancet Gastroenterol Hepatol 2017; 2: 479-93.
13. Brown G J, Daveson J, Marjason J K, et al. A phase I study to determine safety, tolerability and bioactivity of Nexvax2 in HLA DQ2+ volunteers with celiac disease following a long-term, strict gluten-free diet. Gastroenterology 2011; 140: S-437-8.
14. Goel G, Mayassi T, Qiao S-W, et al. Sa1396 A single intradermal (ID) injection of Nexvax2, a peptide composition with dominant epitopes for gluten-reactive CD4+ T cells, activates T cells and tiggers acute gastrointestinal symptoms in HLA-DQ2.5+ people with celiac disease (CeD). Gastroenterology 2016; 150: S304.
15. Burton B R, Britton G J, Fang H, et al. Sequential transcriptional changes dictate safe and effective antigen-specific immunotherapy. Nat Commun 2014; 5: 4741.
16. Burks A W, Calderon M A, Casale T, et al. Update on allergy immunotherapy: American Academy of Allergy, Asthma & Immunology/European Academy of Allergy and Clinical Immunology/PRACTALL consensus report. J Allergy Clin Immunol 2013; 131: 1288-96.e3.
17. Leffler D A, Dennis M, Edwards George J B, et al. A simple validated gluten-free diet adherence survey for adults with celiac disease. Clin Gastroenterol Hepatol 2009; 7: 530-6, 6.e1-2.
18. Svedlund J, Sjodin I, Dotevall G. GSRS—a clinical rating scale for gastrointestinal symptoms in patients with irritable bowel syndrome and peptic ulcer disease. Dig Dis Sci 1988; 33: 129-34.
19. Murray J A, Moore S B, Van Dyke C T, Lahr B D, et al. HLA D Q gene dosage and risk and severity of celiac disease. Clin Gastroenterol Hepatol 2007; 5: 1406-12.
20. Milewski M, Manser K, Nissley B P, Mitra A. Analysis of the absorption kinetics of macromolecules following intradermal and subcutaneous administration. Eur J Pharm Biopharm 2015; 89: 134-44.
21. Kim H S, Patel K G, Orosz E, et al. Time trends in the prevalence of celiac disease and gluten-free diet in the US population: results from the National Health and Nutrition Examination Surveys 2009-2014. JAMA Intern Med 2016; 176: 1716-7.
22. Tye-Din J A, Dzuris J L, Russell A K, Wang S, et al. Serum IL-2 and IL-8 are Elevated within 4 h after Gluten Ingestion in Celiac Disease (CED) Patients on Gluten-Free Diet (GFD) and Potential to Resolve Indeterminate Diagnoses for Patients on GFD. Gastroenterology 152; 5, S114.
23. Kasarda D D. Can an increase in celiac disease be attributed to an increase in the gluten content of wheat as a consequence of wheat breeding? J Agric Food Chem 2013; 61: 1155-9.
24. Hoppe C, Gobel R, Kristensen M, et al. Intake and sources of gluten in 20- to 75-year-old Danish adults: a national dietary survey. Eur J Nutr 2017; 56: 107-17.

### Additional Criteria and Methods for the Studies Described in Example 7

### Study Eligibility Criteria

To be eligible to participate, volunteers must have met the following inclusion criteria and none of the exclusion criteria at the first study visit or at the time indicated.

### Inclusion Criteria

1. Participant is between 18 and 70 years old (inclusive) on the date of the Screening Visit.
2. Participant has been diagnosed with coeliac disease on the basis of intestinal histology showing villous atrophy according to expert guidelines current at the time of diagnosis.
3. Participant has HLA-DQ2.5 genotype (HLA-DQA1*05 and HLA-DQB1*02).

### Exclusion Criteria

1. Participant has not been maintained on a gluten-free diet (gluten-free diet) for at least 1 year.
2. Coeliac Dietary Adherence Test (CDAT) at screening indicates non-compliance to gluten-free diet (score >12).
3. Serum levels of both recombinant human transglutaminase (tTG)-specific IgA (INOVA Diagnostics, San Diego, Calif., USA) and deamidated gliadin peptide-specific IgG (INOVA Diagnostics) are elevated above the manufacturer's upper limit of normal. The elevation of only one of these serology tests is not an exclusion.
4. Participant has uncontrolled complications of coeliac disease or a medical condition which, in the opinion of the investigator, would impact the immune response or pose an increased risk to the participant.
5. Participant is or has been using an immuno-modulatory or immune suppressing medical treatment during the 2 months prior to screening, for example azathioprine, methotrexate, or biological.
6. Participant is female and premenopausal or perimenopausal (<2 years from last menses) and has a male partner who is not sterile (e.g., not vasectomised or not having confirmed azoospermia), unless she is sterile (e.g., bilateral tubal ligation with surgery at least 1 month prior to dosing, hysterectomy, or bilateral oophorectomy with surgery at least 1 month prior to dosing), or she practices true abstinence (when this is in line with her preferred and usual lifestyle), or unless throughout the entire study period and for 30 days after study drug discontinuation she is using a medically acceptable method of contraception (e.g., an intrauterine device, a double-barrier method such as condom with diaphragm, a contraceptive implant, injectable contraceptive, or an oral contraceptive).
7. Participant is male with a premenopausal or perimenopausal (<2 years from last menses) female partner who is not sterile (as defined in exclusion 6), unless he is sterile (e.g., vasectomised or having confirmed azoospermia), or he practices true abstinence (when this is in line with his preferred and usual lifestyle), or unless throughout the entire study period and for 30 days after study drug discontinuation he is using a medically acceptable method of contraception (e.g., a double-barrier method such as condom+partner using diaphragm), or unless his female partner is using a medically acceptable method of contraception (e.g., an intrauterine device, contraceptive implant, injectable contraceptive, or an oral contraceptive).
8. Participant is unable and/or unwilling to comply with study requirements.
9. Participant has taken oral or parenteral corticosteroids (e.g., prednisone, prednisolone, cortisone, or hydrocortisone) within the previous six weeks prior to screening. Topical or inhaled and intranasal corticosteroids are acceptable (e.g., budesonide, fluticasonc, beclomethasone, mometasonc, or triamcinolonc).
10. Participant has received an experimental therapy within 30 days prior to screening.
11. Participant has previously been enrolled and dosed in a clinical trial with Nexvax2®.
12. Participant has any of the following laboratory abnormalities at screening:
   a. Alanine aminotransferase (ALT), aspartate aminotransferase (AST), or alkaline phosphatase (ALP) ≥2× the upper limit of normal (ULN)
   b. Hemoglobin <10 g/dL
   c. Platelet count <100×10⁹/L
   d. White blood cell count (WBC) outside the normal range and judged clinically significant by the investigator
   e. Direct bilirubin outside the normal range
   f. Any other clinically significant abnormal laboratory values, as determined by the investigator
13. Participant is lactating, is known to be pregnant, has a positive pregnancy test at Screening or Treatment Day, intends to become pregnant, or is nursing.
14. Participant has a history or presence of any medically significant condition considered by the investigator to have the potential to adversely affect participation in the study and/or interpretation of the study results.
15. Participant has a history of severe allergic reactions (e.g., swelling of the mouth and throat, difficulty breathing, hypotension, or shock) that require medical intervention.
16. Participant has donated blood ≤56 days prior to screening and plans to donate blood within 5 weeks after study completion.
17. Participant has a clinically relevant abnormality on electrocardiogram (ECG), as determined by the investigator.
18. Other unspecified reasons that in the opinion of the investigator or the sponsor make the participant unsuitable for enrolment.

### Dose Escalation and Stopping Criteria

### Dose Escalation and Down-Dosing

Justification for repeat- or down-dosing was based on the grading of drug-related gastrointestinal symptoms according to Common Terminology Criteria for Adverse Events (CTCAE) if participants experienced mild (Grade 1) or moderate (Grade 2) severity gastrointestinal symptoms. The next higher dose could be administered only if the current dose was tolerated and adverse events were not observed after the third administration of the dose.

The stopping criteria were:
1. Occurrence of SAEs that are judged by the DSMB to be associated with Nexvax2; the DSMB will provide recommendations regarding stopping after each SAE
2. Occurrence of 2 SAEs of the same type judged by the DSMB to be associated with Nexvax2
3. Any AE of Grade 3 or greater severity in 2 or more participants and judged by the DSMB to be associated with Nexvax2
4. Any acute life-threatening response such as anaphylactic reaction or any symptomatic bronchospasm judged to be associated with Nexvax2
5. Hepatotoxicity as defined by ALT >3×ULN accompanied by bilirubin of >2×ULN or an increased direct bilirubin that is ≥2×ULN, and judged to be associated with Nexvax2
6. Moderate or severe myalgia (Grade 2 or higher) will initiate assessment of serum creatine phosphokinase (CPK); levels >6×ULN (≥Grade 2) will result in halting of the study

### Methods

### Investigational Drug Product

CS Bio (Menlo Park, Calif., USA) manufactured the peptides NPL001, NPL002, and NPL003. Grand River Aseptic Manufacturing (Grand Rapids, Mich., USA) formulated and filled vials with a sterile equimolar solution at total peptide concentration 1.5 mg/mL in sterile USP 0.9% sodium chloride. Grand River Aseptic Manufacturing also manufactured the placebo, sterile USP 0.9% sodium chloride, filled in vials identical to active drug. The masked site pharmacist prepared the appropriate dilution of study drug in 0.1 mL using sterile USP 0.9% sodium chloride. For cohorts 1 and 2, each dose was delivered in a single 0.1 mL injection during the escalation phase; during the maintenance phase, each dose was delivered as two equal, divided doses both in 0.1 mL. For cohorts 1 and 2, all injections were administered using fixed needle 1-mL allergy syringes (#30550; Becton-Dickinson, Franklin Lakes, N.J., USA) fitted with a West Intradermal Adapter (#5070206; West Pharmaceutical Services Inc., Exton, Pa., USA). For cohort 3, the first six doses (3 μg to 150 μg) were administered in 0.1 mL by fixed needle 1-mL allergy syringes fitted with a West Intradermal Adapter. The seventh dose was administered as a single injection using a pre-filled Soluvia™ syringe (Becton-Dickinson) containing either 300 μg of Nexvax2 or placebo, which were manufactured by Grand River Aseptic Manufacturing. The eighth through tenth escalation doses of Nexvax2 (450 μg to 750 μg) or placebo were administered as two or three injections using pre-filled Soluvia syringes containing 300 μg of Nexvax2 or placebo, and fixed needle 1-mL allergy syringes fitted with a West Intradermal Adapter containing 150 μg of Nexvax2 or placebo. Maintenance doses in cohort 3 were administered as three injections using pre-filled Soluvia syringes containing 300 μg of Nexvax2 or placebo. The injection site was the abdomen at the level of the waist alternating between the right and left of the body throughout the study.

### Lab Procedures

### Safety Laboratory Pathology Assessments

Laboratory assessments, including routine hematology, blood chemistry, coagulation, and urinalysis, were performed by Dorevitch Pathology (Heidelberg, Victoria, Australia). The following hematology assessments were included: red blood cell count, hemoglobin concentration, hematocrit, platelet count, and white blood cell count with differential (bands, neutrophils, lymphocytes, monocytes, eosinophils, basophils). Blood chemistry included sodium, potassium, chloride, bicarbonate, creatinine, urea, albumin, total protein, alkaline phosphatase (ALP), aspartate transaminase (AST), alanine transaminase (ALT), total bilirubin, and direct bilirubin. Coagulation included prothrombin time (PT) and partial thromboplastin time (PTT). Glucose, calcium, cholesterol, triglycerides, phosphorus, LDH, uric acid, and thyroid-stimulating hormone were measured at the Screening Visit only. Urinalysis was by Dipstick. Urinary pregnancy test (β-hCG) was performed for all female participants.

### Coeliac Disease Serology

Blood was collected into serum tubes, which were allowed to stand upright for 30 minutes at room temperature, and then centrifuged at 1300 g for 10 minutes. Recombinant human transglutaminase 2-specific IgA and deamidated gliadin peptide-specific IgG were measured by Dorevitch Pathology using commercial kits manufactured by INOVA Diagnostics.

### HLA-DQA and HLA-DQB Genotyping and Determination of HLA-DQ2.5 Zygosity

Blood was collected into a K2 EDTA tube. Sonic Genetics (Sonic Healthcare Ltd., Macquarie Park, New South Wales, Australia) determined HLA-DQA and HLA-DQB alleles by polymerase chain reaction and sequence-specific oligonucleotides (Gen-Probe, Hologic Inc., Bedford, Mass., USA). Participants with HLA-DQA1*05 (including all alleles whose numerical code commences with 05 such as HLA-DQA1*0501 or HLA-DQA1*0505) and HLA-DQB1*02 (including all alleles whose numerical code commences with 02 such as HLA-DQB1*0201 or HLA-DQB1*0202) were determined as being HLA-DQ2.5+. Participants who were HLA-DQ2.5+ and had no other HLA-DQA or HLA-DQB alleles were defined as HLA-DQ2.5 homozygotes. All other HLA-DQ2.5+ participants were considered to be HLA-DQ2.5+ non-homozygotes because they possessed additional HLA-DQA and HLA-DQB alleles.

### Anti-Nexvax2 Antibodies

Blood was collected into serum tubes, which were allowed to stand upright for 30 minutes at room temperature, and then centrifuged at 1300 g for 10 minutes. Serum levels of IgG and IgA specific for Nexvax2 peptides (NPL001, NPL002, and NPL003) were analysed by Blue Stream Laboratories, Inc., a Charles River Company (Woburn, Mass., USA). Maleic anhydride activated 96-well plates (#15100; Thermo Fisher Scientific, Grand Island, N.Y., USA) were coated at 4° C. overnight with 100 μL of a mix of six peptides comprising three with sequences identical to NPL001, NPL002, and NPL003, except that a lysl-amide residue was inserted at the C-terminus, and three with sequences identical to NPL001, NPL002, and NPL003, except that the N-terminal residue was replaced by N-glycyl-glutamine (Pepscan Presto BV, Lelystad, Netherlands). The concentration of each peptide in the coating solution was 20 μg/mL in PBS pH 7.4 (#10010; Gibco-Life Technologies, Grand Island, N.Y., USA). Wells were washed 5× with 200 μL of PBS containing 0.1% TWEEN® 20 (#BP337-100; Thermo Fisher Scientific) (pH 7.4). The coated plate was blocked with 200 μL of phosphate buffered saline (PBS) with 1% bovine serum albumin (BSA) (#A3059; Sigma-Aldrich, Natick, Mass., USA), 0.5% TWEEN 20, and 0.5 M glycine (#G7126; Sigma-Aldrich) at pH 7.4 to ensure complete inactivation of any unreacted anhydride moieties. Wells were washed 5× with 200 μL of PBS containing 0.1% TWEEN 20 (pH 7.4). Sera were diluted at 1:500, 1:1000, and 1:2000 in PBS (pH 7.4) with 0.1% BSA and 0.1% TWEEN 20, and 100 μL was added to each of the wells and then incubated for 1 hour at 37° C. Scrum from a healthy human donor diluted 1:500 (for IgG) or 1:1000 (for IgA) in PBS with 0.1% BSA and 0.1% TWEEN 20 served as negative control, and serum from a human donor with untreated coeliac disease served as positive control. Wells were washed 5× with 200 μL of PBS containing 0.1% TWEEN 20 (pH 7.4). For detection of IgG specific for Nexvax2, europium-labelled anti-human IgG (Eu-N1 anti-rabbit IgG (#1244-330; Perkin Elmer, Waltham, Mass., USA) was diluted 1:2500 with PBS (pH 7.4)/0.1% BSA/0.1% TWEEN 20, and 100 μL was added and incubated for 1 hour. For assessment of IgA specific for Nexvax2, rabbit anti-human IgA (#SAB3701232; Sigma-Aldrich) stock (1 mg/mL) was diluted 1:2000 in PBS (pH 7.4)/0.1% BSA/0.1% TWEEN 20, and 100 μL was added to each well. Europium-labelled anti-rabbit IgG (Eu-N1 anti-rabbit IgG; #AD0105; Perkin Elmer) was diluted 1:2500 with PBS (pH 7.4)/0.1% BSA/0.1% TWEEN 20, and 1004, was added and incubated for 1 hour. Wells were washed 5× with 200 μL of PBS containing 0.1% TWEEN 20 (pH 7.4). Liquid was discarded from wells, and then wells were washed 5× with 200 μL of PBS containing 0.1% TWEEN 20 (pH 7.4), and 100 μL of Enhancement Solution (#20114-03; Perkin Elmer) was added to each well, and then incubated at room temperature with shaking for 15 minutes. The plate was then read by time resolved fluorescence (excitation at 360 nm and emission at 615 nm) using a Synergy 1 BioTek Multi-Detection Microplate Reader (BioTek Instruments Inc., Winooski, Vt., USA). The assay was optimised with NPL001/NPL002/NPL003 antisera raised in rabbits following immunization with KLH-NPL001/NPL002/NPL003 conjugates. Cutoff levels were established using 50 individual lots of normal human serum (HemaCare Corporation, Van Nuys, Calif., USA; BioreclamationlVT, Hicksville, N.Y., USA) shown to be seronegative for recombinant human tTG-specific IgA and deamidated gliadin peptide-specific IgG and IgA (INOVA Diagnostics). The upper cutoff was set as the upper 95^{th} percentile, which corresponded to 1194 for Nexvax2-specific IgG and 5754 for Nexvax2-specific IgA.

### Pharmacokinetics

Blood was collected 30 minutes before and 45 minutes after study drug administration. Blood was collected into K2 EDTA tubes and within 10 minutes was centrifuged at 1100-1300 g for 10 minutes. Plasma was aliquotted and frozen. Charles River Laboratories Ashland, LLC (Ashland, Ohio, USA) measured the concentrations of NPL001, NPL002, and NPL003. An ultra-high performance liquid chromatography-mass spectrometry/mass spectrometry (UHPLC-MS/MS) method in the positive electron ionization mode was used for to determine Nexvax2 peptide concentrations in human plasma. Thawed plasma samples (0.3 mL) were spiked with the internal standard, a mixture of isotopically labelled Nexvax2 peptides (Pepscan). A solid phase extraction procedure was used to extract the analyte(s) and internal standard(s). Reconstituted sample extracts were analysed with a UHPLC-MS/MS assay using a Waters Acquity® UPLC Peptide BEH C18 Column, 300 Å, 1.7-μm particle-size, 2.1×50 mm column (Waters Corporation, Milford, Mass., USA). The peak area ratios of NPL001, NPL002, and NPL003, and internal standards and the theoretical concentrations of the calibration samples were fit to a linear regression function with 1/x weighting, excluding the origin. The method was validated over the concentration range of 2.00 to 100 ng/mL of human plasma using a 0.3 mL sample.

### Plasma Concentrations of Cytokines and Chemokines

Blood was collected into K2 EDTA tubes and immediately placed on wet ice. Within 30 minutes of collection, blood was centrifuged at 1100-1300 RCF for 10 minutes, and plasma was aliquotted and frozen. Concentrations of 38 cytokines and chemokines were assessed in thawed plasma at ImmusanT, Inc. (Cambridge, Mass.) using a multiplex magnetic bead assay according to the manufacturer's instructions (Milliplex® MAP Human Cytokine/Chemokine Magnetic Bead Panel; EMD Millipore Corp., Billerica, Mass. and Luminex® MAGPIX® System xPONENT®, Luminex Corporation, Austin, Tex.). Final concentrations were the average of triplicate measurements. An individual participant's plasma sample set was assessed in a single 96-well plate. Pre-treatment cytokine and chemokine concentrations in plasma were compared with post-treatment levels on the same day; other pre-treatment assessments were compared with plasma collected immediately before the first dose was administered.

### Epigenetic Immune Cell Counting

Blood was collected into K2 EDTA tubes and frozen at −20° C. within 60 minutes. Epiontis GmbH (Berlin, Germany) determined the percentage of leukocytes that were T cells (CD3-positive lymphocytes), helper T cells (CD4-positive), cytotoxic T cells (CD8-positive). CCR6-positive T cells, or regulatory T cells (CD3-positive, CD4-positive, CD25-positive, FOXP3-positive) in samples using epigenetic real time PCR based analyses that were unique and highly specific for the cell type of interest measured in the assay.

### Digital Histomorphometry

Four biopsies were collected from the 2^{nd} part of the duodenum using a single pass of the biopsy forceps for each tissue sample. The central pathologist (JiLab Inc., Tampere, Finland) processed and evaluated biopsies. Biopsy samples taken from the distal duodenum were immersed in PAXgene fixative for 1-4 hours and transferred to the proprietary storage solution in PAXgene dual chamber containers (#765112; QIAGEN, Hilden, Germany). Samples were processed as paraffin blocks using a standard formalin-free protocol. Tissue sections (3-4 μm) were cut on SuperFrost Plus slides for hematoxylin and eosin staining. Biopsies were embedded and sections were cut orthogonally to the luminal surface. Immunohistochemistry was performed using a standard protocol consisting of antigen retrieval (incubation at 98° C. for 15 minutes in 0.01 Tris-EDTA buffer, pH 9.0), blocking of endogenous peroxidase (3% H₂O₂ for 5 minutes at RT), primary antibody incubation (60 minutes at RT), anti-mouse or anti-rabbit peroxidase polymer (RTU, 30 minutes at RT, Nichirei Biosciences, Tokyo, Japan), and diamino benzidine chromogen (Nichirei). Slides were counterstained with hematoxylin. The following primary antibodies and dilutions were used: CD3 (clone SP7, 1:100), CD4 (clone SP35 1:100). CD8 (clone C8/144B, 1:100), CD19 (clone LE-CD19, 1:100), CD138 (clone MI15, 1:100), CD163 (clone SP96, 1:100), FOXP3 (clone 5H10L18, 1:100), PD-1 (clone NAT105, 1:100, Cell Marque, Rocklin, Calif., USA). All antibodies except PD-1 were purchased from Thermo Fisher Scientific (Waltham, Mass., USA). Stained slides were scanned as whole slide images using SlideStrider digital slide scanner at resolution 0.28 μm per pixel (Jilab Inc.). Images were stored as JPEG2000 files and viewed with a dedicated web-based Coeliac Slide Viewer (Jilab Inc.). At least three replicate measurements of villus height and crypt depth measurements were done by two independent readers, and the average was used as the final result for villous height to crypt depth ratio. CD3 positive intraepithelial lymphocytes (IELs) and at least 300 enterocytes were enumerated to obtain the IEL count (adjusted per 100 enterocytes). Cells expressing other IHC markers were enumerated and adjusted to three user-defined areas of the lamina propria using the ImmunoRatio2 software, which is part of the Coeliac Slide Viewer.

### Cytokine and Chemokine Gene Expression in Paraffin-Embedded Biopsy Tissue Samples

RNA was extracted from 50 to 100 sections (thickness 3-4 μm) that were cut from the remaining PAXgene tissue block and placed in a test tube by JiLab. In the laboratory of Dr. Keijo Viiri (Center for Child Health Research and Tampere University Hospital, University of Tampere, Tampere, Finland), RNA was extracted using the PAXgene Tissue RNA Kit (#765134, QIAGEN) using an automated robotic nucleic acid extraction system (QIAcube, #9001885, QIAGEN). RNA concentrations were determined with a NanoDrop spectrophotometer and RNA quality with Fragment Analyzer (Advanced Analytical, Ankeny, Iowa, USA) with Standard Sensitivity RNA Analaysis Kit (#DNF-471-0500, Advanced Analytical). Inflammatory gene expression signature of the biopsy samples was analysed using RT2 Profiler PCR Array of Human Cytokines and Chemokines (PAHS-011ZA, #330231, QIAGEN). The array consists of 84 genes listed at https://www.qiagen.com/us/shop/pcdprimer-sets/rt2-profiler-per-arrays/?catno=PAHS-150Z#geneglobe. Genomic DNA was eliminated and cDNA was synthesised by using RT2 First Strand Kit according to the manufacturer's protocol (#330401, QIAGEN). cDNA was synthesised in quadruplicates of 300 ng of RNA per sample after which cDNA was mixed with RT2 SYBR Green Mastermix (#330509, QIAGEN) and loaded into a 384-well array. Each sample was loaded in quadruplicate on one array plate and ran on a Bio-Rad CFX384™ real-time cycler with the cycling conditions recommended by the array manufacturer (PAHS-011ZA, #330231, QIAGEN). Data were analysed with RT2 Profiler PCR Array Data Analysis v3.5 (perdataanalysis.sabiosciences.com/pcdarrayanalysis.php). For each patient, four measurements from the base-line (BL) sample and four measurements from the end-of-study (EOS) sample were analysed. Four measurements were grouped and the data quality was checked. Each group of four measurements passed the PCR Array reproducibility, RT efficiency, and Genomic DNA contamination tests. Gene expression data was normalised to average arithmetic mean of the expressions of ACTB, B2M, GAPDH, HPRT1, and RPLP0 housekeeping genes.

TABLE 1
Demographics and baseline characteristics

| | Treatment | | | | | | | |
|---|---|---|---|---|---|---|---|---|
| | Nexvax2 | | Nexvax2 | | Nexvax2 | | Nexvax2 | |
| | Starting dose, μg | | | | | | | |
| | 30 | | 30 | | 3 | | 3 | |
| | Maintenance dose, μg | | | | | | | |
| | 300 | | 300 | | 300 | | 300 | |
| | Cohort | | | | | | | |
| | 1 | | 2 | | 1 | | 2 | |
| n | 2 | | 4 | | 5 | | 6 | |
| Age (years) | 28 | (27-29) | 42 | (36-43) | 32 | (24-45) | 35 | (32-40) |

Sex

| | | | | | | | | |
|---|---|---|---|---|---|---|---|---|
| Male | 0 | (0%) | 0 | (0%) | 1 | (20%) | 2 | (33%) |
| Female | 2 | (100%) | 4 | (100%) | 4 | (80%) | 4 | (67%) |

Race

| | | | | | | | | |
|---|---|---|---|---|---|---|---|---|
| White | 2 | (100%) | 4 | (100%) | 5 | (100%) | 6 | (100%) |
| Age at diagnosis (years.) | 23 | (21-24) | 35 | (28-39) | 20 | (18-3 [symbol] ) | 30 | (28-31) |
| Time since diagnosis (years) | 6 | (6-7) | 4 | (3-6) | 9 | (4-14) | 8 | (3-11) |
| Time on gluten-free diet (years) | 6 | (6-7) | 4 | (3-6) | 9 | (4-14) | 6 | (3-10) |
| Body mass (kg) | 78 | (71-85) | 61 | (56-66) | 84 | (78-89) | 74 | (60-85) |
| Height (cm) | 169 | (167-170) | 163 | (160-164) | 169 | (168-175) | 168 | (162-177) |
| Body-mass index (kg/m2) | 27 | (25-29) | 24 | (22-25) | 29 | (29-30) | 25 | (21-30) |
| Abnormal serology* | 0 | (0%) | 1 | (25%) | 3 | (40%) | 1 | (17%) |

Homozygote for HLA-DQ2 [symbol] 5 alleles:

| Both | 2 | (100%) | 0 | (0%) | 5 | (100%) | 0 | (0%) |
|---|---|---|---|---|---|---|---|---|
| HLA-DQB1*02 only | 0 | (0%) | 3 | (75%) | 0 | (0%) | 1 | (17%) |
| HLA-DQA1*05 only | 0 | (0%) | 0 | (0%) | 0 | (0%) | 0 | (0%) |
| Neither | 0 | (0%) | 1 | (25%) | 0 | (0%) | 5 | (83%) |
| | Treatment | | | | | | | |
| | Nexvax2 | | Nexvax2 | | Placebo | | Any | |
| | Starting dose, μg | | | | | | | |
| | 3 | | All | | | | All | |
| | Maintenance dose, μg | | | | | | | |
| | 900 | | All | | | | All | |
| | Cohort | | | | | | | |
| | 3 | | All | | All | | All | |
| n | 10 | | 27 | | 9 | | 36 | |
| Age (years) | 53 | (43-60) | 41 | (32-49) | 43 | (32-57) | 41 | (32-53) |

Sex

| | | | | | | | | |
|---|---|---|---|---|---|---|---|---|
| Male | 6 | (60%) | 9 | (33%) | 2 | (22%) | 11 | (31%) |
| Female | 4 | (40%) | 18 | (67%) | 7 | (78%) | 25 | (69%) |

Race

| | | | | | | | | |
|---|---|---|---|---|---|---|---|---|
| White | 10 | (100%) | 27 | (100%) | 9 | (100%) | 30 | (100%) |
| Age at diagnosis (years.) | 39 | (35-46) | 33 | (27-40) | 37 | (30-42) | 34 | (28-41) |
| Time since diagnosis (years) | 7 | (5-12) | 7 | (4-13) | 6 | (2-11) | 7 | (4-12) |
| Time on gluten-free diet (years) | 7 | (5-12) | 6 | (4-12) | 5 | (2-11) | 6 | (3-12) |
| Body mass (kg) | 79 | (69-108) | 73 | (64-90) | 66 | (60-77) | 71 | (62-87) |
| Height (cm) | 175 | (169-181) | 169 | (163-178) | 169 | (165-171) | 169 | (163-175) |
| Body-mass index (kg/m2) | 27 | (26-30) | 26 | (23-30) | 22 | (22-26) | 26 | (22-30) |
| Abnormal serology* | 1 | (10%) | 5 | (19%) | 2 | (22%) | 7 | (19%) |

Homozygote for HLA-DQ2 [symbol] 5 alleles:

| | | | | | | | | |
|---|---|---|---|---|---|---|---|---|
| Both | 0 | (0%) | 7 | (26%) | 3 | (33%) | 10 | (28%) |
| HLA-DQB1*02 only | 4 | (40%) | 8 | (30%) | 1 | (11%) | 9 | (25%) |
| HLA-DQA1*05 only | 1 | (10%) | 1 | (4%) | 0 | (0%) | 1 | (3%) |
| Neither | 5 | (50%) | 11 | (41%) | 5 | (5 [symbol] %) | 16 | (44%) |

Data are median (IQK) or n (%).
*Deamidated gliadin peptide IgG or transglutaminase 2 IgA. [symbol]
indicates data missing or illegible when filed

TABLE 2
Overall adverse events summary for participants starting at 3 μg or 30 μg of Nexvax2

| | Treatment | | | | | | | | | | | |
|---|---|---|---|---|---|---|---|---|---|---|---|---|
| | Nexvax2 | | Nexvax2 | | Nexvax2 | | Nexvax2 | | Nexvax2 | | Placebo | |
| | Starting dose, μg | | | | | | | | | | | |
| | 30 | | 30 | | 3 | | 3 | | 3 | | | |
| | Maintenance dose, μg | | | | | | | | | | | |
| | 300 | | 300 | | 300 | | 300 | | 900 | | | |
| | Cohort | | | | | | | | | | | |
| | 1 | | 2 | | 1 | | 2 | | 3 | | All | |
| Participants, n | 2 | | 4 | | 5 | | 6 | | 10 | | 9 | |
| Participants with any adverse events | 2 | (100%) | 4 | (100%) | 3 | (60%) | 6 | (100%) | 9 | (90%) | 9 | (100%) |
| Participants with any drug-related adverse events. | 2 | (100%) | 4 | (100%) | 3 | (60%) | 6 | (100%) | 7 | (73%) | 8 | (89%) |
| Participants with any adverse events graded at least | 2 | (100%) | 3 | (75%) | 2 | (40%) | 5 | (83%) | 6 | (60%) | 4 | (44%) |
| moderate in severity | | | | | | | | | | | | |
| Participants with any adverse events graded at least | 1 | (50%) | 2 | (50%) | 1 | (20%) | 4 | (67%) | 2 | (20%) | 2 | (22%) |
| moderate in severity and drug-related | | | | | | | | | | | | |
| Participants who withdrew due to adverse events | 1 | (50%) | 0 | (0%) | 0 | (0%) | 0 | (0%) | 0 | (0%) | 1 | (11%) |
| Participants with any serious adverse events | 0 | (0%) | 0 | (0%) | 0 | (0%) | 0 | (0%) | 0 | (0%) | 2 | (22%) |

| | | | | | | |
|---|---|---|---|---|---|---|
| Adverse events | 34 | 57 | 16 | 56 | 44 | 46 |
| Adverse events drug-related | 21 | 45 | 9 | 41 | 30 | 25 |
| Adverse events graded at least moderate in severity | 7 | 5 | 3 | 17 | 12 | 7 |
| Adverse events graded at least moderate in severity | 5 | 2 | 1 | 13 | 2 | 4 |
| and drug-related | | | | | | |
| Adverse events leading to withdrawal | 1 | 0 | 0 | 0 | 0 | 1 |
| Serious adverse events | 0 | 0 | 0 | 0 | 0 | 2 |

Data ate n (%).

TABLE 3
Adverse events by system organ class for participants starting at 3 μg or 30 μg of Nexvax2

| | Treatment | | | | | |
|---|---|---|---|---|---|---|
| | Nexvax2 | Nexvax2 | Nexvax2 | Nexvax2 | Nexvax2 | |
| | Starting dose, μg | | | | | |
| | 30 | 30 | 3 | 3 | 3 | |
| | Maintenance dose, μg | | | | | |
| | 300 | 300 | 300 | 300 | 900 | Placebo |
| | Cohort | | | | | |
| | 1 | 2 | 1 | 2 | 3 | All |
| Participants, n | 2 | 4 | 5 | 6 | 10 | 9 |
| Any adverse events | 2 (100%) 34 | 4 (100%) 57 | 3 (60%) 16 | 6 (100%) 56 | 9 (90%) 44 | 9 (100%) 46 |
| Gastrointestinal disorders | 2 (100%) 11 | 4 (100%) 28 | 3 (60%) 5 | 6 (100%) 2 [symbol] | 7 (70%) 13 | 6 (67%) 14 |
| Diarrhea | 1 (50%) 1 | 2 (50%) 2 | 1 (20%) 1 | 5 (83%) 9 | 4 (40%) 5 | 1 (11%) 1 |
| Nausea | 2 (100%) 4 | 3 (75%) 10 | 1 (20%) 1 | 2 (33%) 3 | 2 (20%) 2 | 3 (33%) 4 |
| Abdominal pain | 1 (50%) 2 | 1 (25%) 1 | 0 (0%) 0 | 3 (50%) 0 | 2 (20%) 3 | 0(0%) 0 |
| Abdominal pain upper | 1 (50%) 1 | 0 (0%) 0 | 0 (0%) 0 | 0 (0%) 0 | 0 (0%) 0 | 0 (0%) 0 |
| Abdominal pain lower | 0 (0%) 0 | 1 (25%) 1 | 0 (0%) 0 | 0 (0%) 0 | 0 (0%) 0 | 0 (0%) 0 |
| Abdominal discomfort | 0 (0%) 0 | 2 (50%) 3 | 1 (20%) 1 | 2 (33%) 3 | 0 (0%) 0 | 2 (22%) 4 |
| Gastroesophageal reflux | 1 (50%) 2 | 1 (25%) 1 | 1 (20%) 1 | 0 (0%) 0 | 1 (10%) 1 | 0 (0%) 0 |
| Fistul [symbol] | 0 (0%) 0 | 1 (25%) 1 | 0 (0%) 0 | 0 (0%) 0 | 1 (10%) 1 | 1 (11%) 1 |
| Abdominal distension | 1 (50%) 1 | 1 (25%) 3 | 1 (20%) 1 | 1 (17%) 1 | 0 (0%) 0 | 2 (22%) 2 |
| Eructation | 0 (0%) 0 | 2 (50%) 5 | 0 (0%) 0 | 0 (0%) 0 | 0 (0%) 0 | 0 (0%) 0 |
| Vomiting | 0 (0%) 0 | 1 (25%) 1 | 0 (0%) 0 | 0 (0%) 0 | 0 (0%) 0 | 1 (11%) 1 |
| Constipation | 0 (0%) 0 | 0 (0%) 0 | 0 (0%) 0 | 0 (0%) 0 | 0 (0%) 0 | 1 (11%) 1 |
| Nervous system disorders | 1 (50%) 3 | 4 (100%) 8 | 2 (40%) 3 | 4 (67%) 11 | 6 (60%) 9 | 3 (33%) 6 |
| Headache | 0 (0%) 0 | 2 (50%) 3 | 2 (40%) 2 | 4 (67%) 9 | 6 (60%) 8 | 1 (11%) 1 |
| Migraine | 0 (0%) 0 | 0 (0%) 0 | 0 (0%) 0 | 1 (17%) 1 | 0 (0%) 0 | 0 (0%) 0 |
| Tension headache | 0 (0%) 0 | 0 (0%) 0 | 0 (0%) 0 | 0 (0%) 0 | 1 (10%) 1 | 0 (0%) 0 |
| Dizziness | 1 (50%) 3 | 1 (25%) 1 | 0 (0%) 0 | 0 (0%) 0 | 0 (0%) 0 | 1 (11%) 1 |
| Dysgeusia | 0 (0%) 0 | 2 (50%) 2 | 0 (0%) 0 | 0 (0%) 0 | 0 (0%) 0 | 0 (0%) 0 |
| Lethargy | 0 (0%) 0 | 0 (0%) 0 | 0 (0%) 0 | 1 (17%) 1 | 0 (0%) 0 | 2 (22%) 2 |
| Syncope | 1 (50%) 1 | 0 (0%) 0 | 0 (0%) 0 | 0 (0%) 0 | 0 (0%) 0 | 0 (0%) 0 |
| General disorders & | 2 (100%) 13 | 3 (75%) 15 | 1 (20%) 4 | 3 (50%) 4 | 2 (20%) 4 | 3 (33%) 11 |
| administration site conditions | | | | | | |
| Fatigue | 1 (50%) 2 | 2 (50%) 6 | 1 (20%) 1 | 1 (17%) 2 | 0 (0%) 0 | 2 (22%) 4 |
| Injection site reactions | 2 (100%) 6 | 2 (50%) 6 | 1 (20%) 1 | 2 (33%) 2 | 2 (20%) 2 | 2 (22%) 3 |
| Injection site erythema | 1 (50%) 4 | 2 (50%) 5 | 0 (0%) 0 | 0 (0%) 0 | 1 (10%) 1 | 2 (22%) 2 |
| Injection site pruritus | 1 (50%) 1 | 1 (25%) 1 | 0 (0%) 0 | 1 (17%) 1 | 0 (0%) 0 | 0 (0%) 0 |
| Injection site pain | 1 (50%) 1 | 0 (0%) 0 | 1 ( [symbol] 0%) 1 | 0 (0%) 0 | 0 (0%) 0 | 0 (0%) 0 |
| Injection site reaction | 0 (0%) 0 | 0 (0%) 0 | 0 (0%) 0 | 1 (17%) 1 | 1 (10%) 1 | 0 (0%) 0 |
| Injection site bruise | 0 (0%) 0 | 0 (0%) 0 | 0 (0%) 0 | 0 (0%) 0 | 0 (0%) 0 | 1 (11%) 1 |
| Skin & subcutaneous | 2 (100%) 4 | 1 (25%) 1 | 0 (0%) 0 | 2 (33%) 3 | 4 (40%) 4 | 0 (0%) 0 |
| tissue disorders | | | | | | |
| Ecchymosis | 0 (0%) 0 | 0 (0%) 0 | 0 (0%) 0 | 1 (17%) 2 | 1 (10%) 1 | 0 (0%) 0 |
| Infections and infestations | 1 (50%) 1 | 1 (25%) 1 | 1 (20%) 1 | 1 (17%) 1 | 4 (40%) [symbol] | 1 (11%) 1 |
| URTT | 0 (0%) 0 | 1 (25%) 1 | 0 (0%) 0 | 0 (0%) 0 | 2 (20%) 5 | 1 (11%) 1 |
| Musculoskeletal & | 1 (50%) 1 | 1 (25%) 2 | 0 (0%) 0 | 1 (17%) 1 | 3 (30%) 3 | 5 (56%) [symbol] |
| connective tissue disorders | | | | | | |
| Arthralgia | 1 (50%) 1 | 1 (25%) 1 | 0 (0%) 0 | 1 (17%) 1 | 1 (10%) 1 | 1 (11%) 1 |
| Back pain | 0 (0%) 0 | 0 (0%) 0 | 0 (0%) 0 | 0 (0%) 0 | 1 (10%) 1 | 2 (22%) 2 |
| Musculoskeletal pain | 0 (0%) 0 | 0 (0%) 0 | 0 (0%) 0 | 0 (0%) 0 | 0 (0%) 0 | 2 (22%) 3 |
| Injury, poisoning & | 0 (0%) 0 | 0 (0%) 0 | 1 (20%) 2 | 3 (50%) 3 | 0 (0%) 0 | 3 (33%) 4 |
| procedural complications | | | | | | |
| Contusion | 0 (0%) 0 | 0 (0%) 0 | 0 (0%) 0 | 1 (17%) 1 | 0 (0%) 0 | 2 (22%) 2 |
| Vascular disorders | 0 (0%) 0 | 1 (25%) 1 | 0 (0%) 0 | 2 (33%) 2 | 1 (10%) 1 | 0 (0%) 0 |
| Phlebitis | 0 (0%) 0 | 0 (0%) 0 | 0 (0%) 0 | [symbol] (33%) 2 | 0 (0%) 0 | 0 (0%) 0 |

Data are n (%) and total number adverse events. Treatment-emergent adverse events are shown only if reported by more than one participant. [symbol]
indicates data missing or illegible when filed

### Example 8. Randomized, Double-Blind, Placebo-Controlled Study in HLA-DQ2.5+ Adults with Celiac Disease to Assess the Effect of Nexvax2 on Symptoms after Masked Gluten Food Challenge

### Study Rationale

This example is of Nexvax2 as a self-administered maintenance therapy for patients with CeD who are positive for HLA-DQ2.5.

The effects of Nexvax2 administered intradermally (ID) and subcutaneously (SQ) have been assessed in preclinical studies and in 106 HLA-DQ2.5+ CeD patients on gluten-free diet (GFD) administered Nexvax2 in completed Phase 1 studies. A treatment regimen of updosing starting at 3 μg followed by maintenance dosing 900 μg has been established. This study evaluates the possible outcome that GI symptoms and immune activation after gluten food challenge (FC) are reduced in HLA DQ2.5+ patients with CeD on a GFD who receive Nexvax2 compared with those who receive placebo.

Phase 1 studies have assessed CeD patients separately according to whether they are homozygous for CeD-susceptibility alleles of both genes encoding HLA-DQ2.5 (HLA-DQA1*05 and HLA-DQB1′02). CeD patients with two copies of both HLA-DQA1*05 and HLA-DQB1*02 (“HLA-DQ2.5 homozygous”) are assessed at dose levels above 300 μg, and HLA-DQ2.5 non-homozygous CeD patients have received dose levels as high as 900 μg. For this reason, HLA-DQ2.5 homozygotes are randomized into a separate exploratory cohort, emphasizing assessment of safety and tolerability.

The primary endpoint is based on assessments of self-reported GI symptoms after patients consume gluten in a bolus sham-controlled masked food challenge (MFC) compared to symptoms they reported in the baseline pre-treatment interval. Inclusion of a sham FC is intended to reduce the nocebo effect of gluten FC, and a second MFC is used to assess whether the effects of Nexvax2 treatment persist upon gluten re-exposure. Serum cytokines are also assessed after the FCs to assess levels of systemic immune activation caused by eating gluten, and to explore the correlation between serum levels of cytokines, especially IL-2, and severity of symptoms recorded by the Celiac Disease Patient-reported Outcome (CeD PRO®), which may eventually provide a quantitative surrogate marker for both symptoms and immune activation caused by gluten. A subset of patients have endoscopies before treatment and near the end of treatment to compare changes in duodenal histology across treatment groups.

The initial indication for Nexvax2 is intended to be protection against symptoms caused by inadvertent gluten exposure in CeD patients positive for HLA-DQ2.5 and following a GFD. To focus the clinical development of Nexvax2 on the target population of CeD patients who are most likely to benefit from Nexvax2 treatment, this study incorporates a single unmasked gluten FC on the first day of screening to identify and exclusively randomize patients who experience GI symptoms after ingesting gluten.

### Rationale for Dose and Regimen

Overall, a 2-times-per-week ID administration regimen was established. Further, the results of one study showed that doses up to 900 μg preceded by an updosing phase (starting at 3 μg) were safe and well tolerated by HLA DQ2.5 non-homozygous patients (Cohort 3) and that doses up to 300 μg preceded by an updosing phase (starting at 3 μg) were safe and well tolerated by HLA DQ2.5 homozygous patients (Cohort 1). No dose limiting toxicity was observed with Nexvax2 during updosing or at the respective maintenance dose in HLA-DQ2.5 homozygous or non-homozygous patients with CeD. Pharmacodynamics (PD) results were consistent with the development of gluten peptide-specific immune non-responsiveness.

In this study, the first dose during updosing is 1 μg, which is followed by the same 10 dose increments (3 to 750 μg) and maintenance (900 μg) dose levels as described herein. The maintenance dose level of 900 μg administered 2 times weekly (after updosing) is selected because of its safety and tolerability, and also because “non-responsiveness” to this dose level in patients after updosing over 5 weeks suggests that immune activation following ingestion of bolus FC containing 6 g gluten would be reduced by regular administration of Nexvax2 900 μg. In fact, the “antigenic strength” of Nexvax2 900 μg is likely to be substantially greater than the amounts of gluten typically consumed by Americans (˜14 g daily). This conclusion is also supported by the finding that serum levels of IL-2, a marker of T cell activation, increase in CeD patients on GFD after the first dose of Nexvax2 150 μg to median levels that are about 6 times higher than those stimulated by eating 3 g of gluten (Tye Din et al. 2017).

Nexvax2 is administered SQ in this study.

Maintenance doses of Nexvax2 (or matched placebo) are self-administered using a pre-filled, disposable autoinjector (BD Physioject™). The BD Physioject™ allows precise dosing while eliminating the need for patients to travel to the study site during each visit within the maintenance phase of the treatment period.

The interval between the penultimate (i.e., second-to-last) and final maintenance doses of Nexvax2 is 1 week to allow assessment of the clinical and immunological effects of this longer dose interval during “long-term” maintenance. In preclinical studies, immunological non responsiveness to Nexvax2 was maintained by once weekly SQ dosing; in addition, 1-week dose intervals were assessed in other Nexvax2 Studies in which a total of 3 fixed doses were administered.

### Rationale for Treatment Duration

The PK of Nexvax2 at the maximum dose level planned for this study was (Cohort 3). The results showed no drug accumulation when the 900 μg maintenance dose was administered 8 times over 4 weeks.

The results also showed that immunological non-responsiveness is partially achieved after 2 weeks of therapy, while there is no measurable immune activation triggered by systemic exposure to Nexvax2 peptides after 2 months of therapy. A possible outcome is that longer duration of therapy induces more robust immunological non-responsiveness. In turn, clinical tolerance to gluten exposure requires establishing robust immunological non responsiveness. A total treatment duration of approximately 16 weeks (4 months) was chosen for this study, with approximately 3 months of therapy to induce immunological non responsiveness prior to the initiation of the MFCs, some of which contain gluten.

### Rationale for Choice of Comparator

The control groups (Arms B and D) are given placebo because no approved pharmacological therapy is available as an active comparator to Nexvax2. The only management available for CeD is a GFD. Nexvax2 and placebo are given to patients with CeD on a GFD. Thus, all patients maintain their GFD throughout the study, apart from the unmasked gluten FC during screening and up to 2 of the 3 MFCs during the treatment period.

### Rationale for Gluten Food Challenge Amount and Duration

Gluten boluses are ingested 1 time during the screening period and at least 1 but no more than 2 times during the 3 MFCs during the treatment period for a given patient. Since gluten may provoke ill-defined systemic symptoms rather than GI symptoms in some CeD patients, the unmasked gluten challenge on the first day of screening serves to identify and exclude participants who do not report an increase in overall GI symptoms after consuming gluten.

The amount of gluten protein ingested in each FC containing gluten is approximately 6 g, calculated by the Osbourne method (Hoppe et al. Intake and sources of gluten in 20- to 75-year-old Danish adults: a national dietary survey. Eur J Nutr 56, 107-17 (2017)), which compares to average daily gluten ingestion of about 14 g by Americans (Kasarda. Can an increase in celiac disease be attributed to an increase in the gluten content of wheat as a consequence of wheat breeding? J Agric Food Chem 61, 1155-9 (2013)). Administering gluten at this level daily for periods as long as 6 to 12 weeks has been regarded as a moderate gluten challenge (Ländeaho et al. Small-bowel mucosal changes and antibody responses after low- and moderate-dose gluten challenge in celiac disease. BMC Gastroenterol 11, 129 (2011); Ländeaho et al Glutenase ALV003 attenuates gluten-induced mucosal injury in patients with celiac disease. Gastroenterol 146, 1649-1658 (2014)). An FC for 3 days in CeD patients on a GFD does not cause intestinal injury but does transiently reactivate gluten-specific T cells (Brottveit et al. Assessing possible celiac disease by an HLA-DQ2-gliadin Tetramer Test. Am J Gastroenterol 106, 1318-24 (2011)). GI symptoms show a trend towards worsening at 6 hours after initial ingestion of a moderate FC and can result in abdominal symptoms of pain, nausea, rumbling, bloating, and diarrhea that resolve by the following day when gluten is discontinued (Sarna et al. HLA-DQ:gluten tetramer test in blood gives better detection of coeliac patients than biopsy after 14-day gluten challenge. Gut pii: gutjnl-2017-314461 (2017); Goel et al. Epitope-specific immunotherapy targeting CD4 positive T cells in coeliac disease: two randomised, double-blind, placebo-controlled phase 1 studies. Lancet Gastroenterol Hepatol 2, 479-493 (2017)).

Between 2 and 6 hours after an FC with a liquid slurry of vital wheat gluten estimated to contain 3 g of gluten or after ingestion of wheat bread estimated to contain 6 g of gluten, elevations of circulating levels of IL-2, IL-8, and IL-10 as well as CCL20 have been observed (Tye Din et al. Gluten ingestion and intradermal injection of peptides that activate gluten-specific CD4+ T cells elicit a cytokine signature dominated by interleukin-2 in celiac disease. United European Gastroenterol J 5, A26-27 (2017); unpublished). Serum levels of cytokines are tested at 2, 4, and 6 hours following the screening food challenge (SFC) and at 4 hours following each MFC in order to understand whether cytokine elevations are correlated with severity of symptoms.

### Study Design

### Overview of Study Design

This study is a Phase 2, randomized, double-blind, placebo-controlled clinical study of Nexvax2, a peptide-based therapeutic vaccine, in HLA DQ2.5+ adult patients with confirmed CeD who initiated a GFD at least 12 months prior to screening. The primary study population is comprised of HLA-DQ2.5 non-homozygotes (target randomization of 128). A small and separate exploratory cohort of HLA-DQ2.5 homozygotes (target randomization of 18) is also enrolled. The study evaluates the efficacy of SQ administered Nexvax2 (900 μg) compared with matched placebo (Arms A and B, respectively, for HLA-DQ2.5 non-homozygotes, and C and D, respectively, for the exploratory cohort of HLA-DQ2.5 homozygotes). The primary measure of efficacy is symptoms when a limited and defined MFC containing gluten is given as a bolus within the last 5 weeks of treatment. The study also assesses safety, and tolerability of Nexvax2 in HLA-DQ2.5 non-homozygotes (Arms A and B) and HLA-DQ2.5 homozygotes (Arms C and D). In a subset of HLA-DQ2.5 non-homozygous patients, the effects of Nexvax2 on duodenal histology compared to placebo are also assessed by upper GI endoscopy with second part duodenal biopsies to measure quantitative histology before and after treatment.

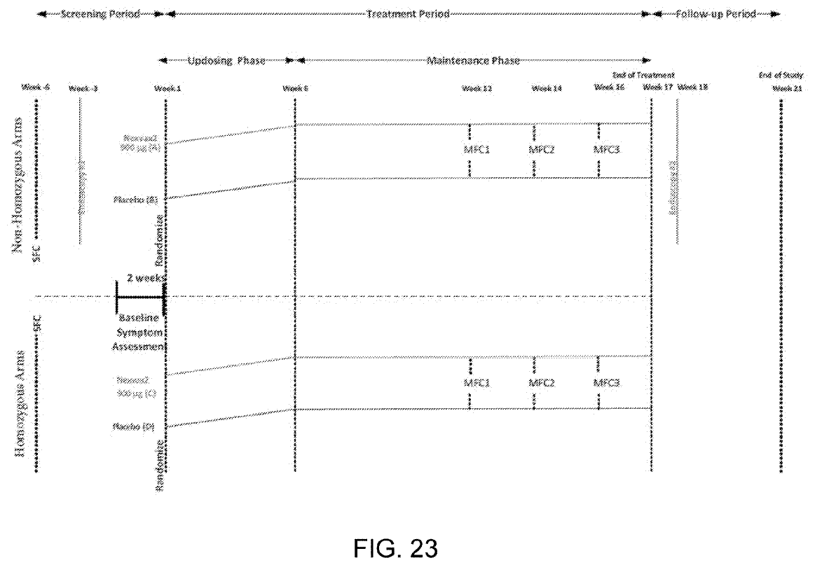

The study design is summarized in FIG. 23, and the timing of specific assessments is provided in the Schedule of Assessments (SoA) (Table 4).

The study plan consists of 3 phases: a screening period of 6 weeks (including an unmasked FC containing gluten on the first day), an approximately 16 week treatment period (including 3 MFCs, with at least 1 and no more than 2 containing gluten), and a 4-week post-treatment observational follow-up period.

The primary efficacy endpoint is based on results from the HLA-DQ2.5 non-homozygote cohort's responses on the CeD PRO instrument, in particular, the change for a patient in their Total GI Domain score for the day of the first MFC containing gluten from their baseline over the 14 days prior to the treatment period. The CeD PRO is collected daily from screening through the end of treatment (EOT) using a patient-handheld device.

On the first day of screening, patients who meet initial eligibility criteria are enrolled and have further clinical assessments, blood tests, and then an unmasked screening food challenge (SFC) with vital wheat gluten flour (containing ˜6 g gluten protein) in water followed by a 6 hour observation period. Patients who meet all inclusion and none of the exclusion criteria, including the criteria for randomization, are randomized in a 1:1 ratio to Arms A or B for HLA-DQ2.5 non-homozygotes, or in a 2:1 ratio to Arms C or D for HLA-DQ2.5 homozygotes, with Arms A and C receiving Nexvax2 and Arms B and D receiving placebo. Patients are excluded before randomization to treatment if they do not experience worsening GI symptoms after the SFC.

Randomization to Arm A versus Arm B, or to Arm C versus Arm D, is blinded. All patients receiving Nexvax2 have updosing starting from 1 μg with 11 stepwise doses before reaching the maintenance dose of 900 μg (all by SQ administration). All Nexvax2 is administered 2 times per week except the last dose, which follows 1 week after the penultimate dose.

The MFCs during the treatment period are double blind. Patients are randomized to a pre-defined sequence of gluten-containing or sham MFCs during the treatment period. At least 1 and no more than 2 MFCs per patient contain gluten.

With the exception of protocol-specified gluten consumption at the SFC and MFCs, patients continue adhering to their established, pre-enrollment GFD. During visits for extended periods to the study site, patients bring their own gluten-free food for consumption.

Patients who withdraw from the study prematurely are not replaced.

The total duration of study participation for an individual patient is typically approximately 26 weeks. Patients may have additional updosing as unscheduled visits, for a total of up to approximately 37 weeks of study participation.

A total of 146 patients are randomized. Approximately 256 patients are screened. Patients are randomized in a 1:1 ratio to the Nexvax2:placebo treatment arms for HLA-DQ2.5 non-homozygotes, or 2:1 ratio to the Nexvax2:placebo treatment arms for HLA DQ2.5 homozygotes.

Approximately 25 HLA-DQ2.5 non-homozygous patients per treatment arm are included in the subset assessed by upper GI endoscopy with second part duodenal biopsies.

### Study Periods

The duration of study participation is approximately 26 weeks, including the 42-day (6-week) screening period, 113-day (approximately 16-week) treatment period, and 28-day (4-week) observational follow-up period. Patients may have up to an additional 11 weeks of updosing as unscheduled visits during the treatment period, for a total of approximately 37 weeks of study participation. The location of visits (study site or patient's home) is specified in the SoA (Table 4).

TABLE 4
Schedule of Assessments for Study Nexvax2-2006

| | Screening Period | | | | Treatment Period | | | | | | | | | | | |
|---|---|---|---|---|---|---|---|---|---|---|---|---|---|---|---|---|
| | V1 | | V-EGD | | Updosing Phase | | | | | | | | | | | |
| Visit | (SFC) | V2 | 1^{a} | V3 | V4 | V5^{b} | V6^{b} | V7^{b} | V8^{b} | V9^{b} | V10^{b} | V11^{b} | V12^{b} | V13^{b} | V14^{b} | V15^{b} |
| Day | −42 | −21 | −21 | −14 | −7 | 1 | 4 | 8 | 11 | 15 | 18 | 22 | 25 | 29 | 32 | 36 |
| | | | to −14 | | | | | | | | | | | | | |
| Week | −6 | −3 | −3 | −2 | −1 | 1 | 1 | 2 | 2 | 3 | 3 | 4 | 4 | 5 | 5 | 6 |
| Nexvax2 (Arm A & C) | | | | | | 1 | 3 | 9 | 30 | 60 | 90 | 150 | 300 | 450 | 600 | 750 |
| (μg) | | | | | | | | | | | | | | | | |
| Placebo (Arm B & D) | | | | | | 0 | 0 | 0 | 0 | 0 | 0 | 0 | 0 | 0 | 0 | 0 |
| (μg) | | | | | | | | | | | | | | | | |
| Dose Number | | | | | | 1 | 2 | 3 | 4 | 5 | 6 | 7 | 8 | 9 | 10 | 11 |

Visit Locations

| | | | | | | | | | | | | | | | | |
|---|---|---|---|---|---|---|---|---|---|---|---|---|---|---|---|---|
| Study Site | X | | (X) | | | X | X | X | X | X | X | X | X | X | X | X |
| Patient's Home | | X | | X | X | | | | | | | | | | | |

Administrative Procedures

| | | | | | | | | | | | | | |
|---|---|---|---|---|---|---|---|---|---|---|---|---|---|
| Informed Consent^{c} | X | | | | | | | | | | | | |
| Inclusion/Exclusion | X | | X | | | | | | | | | | |
| Criteria^{d} | | | | | | | | | | | | | |
| Randomization | | | X | | | | | | | | | | |
| Demographics | X | | | | | | | | | | | | |
| Medical/Surgical | X | | | | | | | | | | | | |
| History^{e} | | | | | | | | | | | | | |
| Celiac Disease | X | | | | | | | | | | | | |
| Diagnosis^{f} | | | | | | | | | | | | | |
| Clinical | X | | | | | | | | | | | | |
| Characteristics of | | | | | | | | | | | | | |
| Celiac Disease^{e} | | | | | | | | | | | | | |
| Prior/Concomitant | X | | X | X | X | X | X | X | X | X | X | X | X |
| Medications^{g} | | | | | | | | | | | | | |
| CDAT | X | | | | | | | | | | | | |
| Compliant with GFD: | X | (X) | X | X | X | X | X | X | X | X | X | X | X |
| Yes/No^{h} | | | | | | | | | | | | | |
| IGFD | X | | | | | | | | | | | | |
| HLA-DQA and | X | | | | | | | | | | | | |
| HLA-DQB | | | | | | | | | | | | | |
| GLOSSⁱ | X, 1 h, | | | | | | | | | | | | |
| | 2 h, 3 h, | | | | | | | | | | | | |
| | 4 h, 5 h, | | | | | | | | | | | | |
| | 6 h | | | | | | | | | | | | |
| Modified CeD | X, 1 h, | | | | | | | | | | | | |
| PROⁱ | 2 h, 3 h, | | | | | | | | | | | | |
| | 4 h, 5 h, | | | | | | | | | | | | |
| | 6 h | | | | | | | | | | | | |

Clinical Procedures

| | | | | | | | | | | | | |
|---|---|---|---|---|---|---|---|---|---|---|---|---|
| Vital Signs^{j} | X | X, 4 h | X | X | X | X | X | X | X | X | X | X |
| Weight | X | | | | | | | | | | | |
| IIeight | X | | | | | | | | | | | |
| Physical Examination^{k} | X | | | | | | | | | | | |
| 12-lead ECG^{l} | X | X | | | | | | | | | | |

Clinical Procedures (cont)

| | | | | | | | | | | | | | |
|---|---|---|---|---|---|---|---|---|---|---|---|---|---|
| Adverse Event | X | | X | X | X | X | X | X | X | X | X | X | X |
| Monitoring^{m} | | | | | | | | | | | | | |
| Endoscopy/Duodenal | | (X) | | | | | | | | | | | |
| Biopsy^{a} | | | | | | | | | | | | | |

Clinical Outcome Assessments

| | | | | | | | | | | | | | | | | |
|---|---|---|---|---|---|---|---|---|---|---|---|---|---|---|---|---|
| Provide and/or Collect | X | | | | | Xⁿ | | | | | | | | | | |
| ePRO Device | | | | | | | | | | | | | | | | |
| Daily CeD PROⁱ | | X | X | X | X | X | X | X | X | X | X | X | X | X | X | X |
| BSFS + PGA-BFⁱ | | X | (X) | X | X | X | X | X | X | X | X | X | X | X | X | X |
| PGA-Sⁱ | | | | | X | | | | | | | | | | | |
| CGA^{o} | | | | | | X | | | | | | | | | | |
| ICDSQⁱ | | | | | X | | | | | | | | | | | |
| SF-12v2ⁱ | | | | | X | | | | | | | | | | | |

Laboratory Assessments

| | | |
|---|---|---|
| Hematology/ | X | X |
| Coagulation | | |
| Blood Chemistry 1 | X | X |
| Blood Chemistry 2 | X | X |
| Urinalysis^{p} | X | X |
| Pregnancy Testing^{q} | X | X |
| Serum Celiac Disease | X | X |
| Serology^{r} | | |
| Exposure | | |
| Pharmacokinetics^{s} | | |
| Serum Anti-Nexvax2 | | X |
| Antibodies | | |
| Serum Cytokines | X, 2 h, | X, 4 h |
| (IL-2, IL-8, | 4 h, 6 h | |
| IL-10, and | | |
| CCL20)^{t} | | |

Administration of IP

| | | | | | | | | | | | |
|---|---|---|---|---|---|---|---|---|---|---|---|
| IP Administration by | X | X | X | X | X | X | X | X | X | X | X |
| Site Staff | | | | | | | | | | | |
| Patient Self- | | | | | | | | | | | |
| administration of IP | | | | | | | | | | | |
| Return of Pre- | | | | | | | | | | | |
| filled Syringes | | | | | | | | | | | |

FC Procedure

| | |
|---|---|
| Unmasked Gluten FC^{u} | X |
| Masked Gluten/Sham | |
| FC^{u} | |

| | Treatment Period | | | | | | | | | | | | | | | |
|---|---|---|---|---|---|---|---|---|---|---|---|---|---|---|---|---|
| | Maintenance Phase (First Part) | | | | | | | | | | | | | | | |
| | | | | | | | | | | | | | V28 | | | |
| | | | | | | | | | | | | | (MF | | | |
| Visit | V16 | V17 | V18 | V19 | V20 | V21 | V22 | V23 | V24 | V25 | V26 | V27 | C1) | V29 | V30 | V31 |
| Day | 39 | 43 | 46 | 50 | 53 | 57 | 60 | 64 | 67 | 71 | 74 | 78 | 79 | 80 | 81 | 85 |
| Week | 6 | 7 | 7 | 8 | 8 | 9 | 9 | 10 | 10 | 11 | 11 | 12 | 12 | 12 | 12 | 13 |
| Nexvax2 (Arm A & C) | 900 | 900 | 900 | 900 | 900 | 900 | 900 | 900 | 900 | 900 | 900 | 900 | | | 900 | 900 |
| (μg) | | | | | | | | | | | | | | | | |
| Placebo (Arm B & D) | 0 | 0 | 0 | 0 | 0 | 0 | 0 | 0 | 0 | 0 | 0 | 0 | | | 0 | 0 |
| (μg) | | | | | | | | | | | | | | | | |
| Dose Number | 12 | 13 | 14 | 15 | 16 | 17 | 18 | 19 | 20 | 21 | 22 | 23 | | | 24 | 25 |

Visit Locations

| | | | | | | | | | | | | | | | | |
|---|---|---|---|---|---|---|---|---|---|---|---|---|---|---|---|---|
| Study Site | X | | | PC^{v} | | | | | | X | | | X | | | |
| Patient's Home | | X | X | X | X | X | X | X | X | | X | X | | X | X | X |

Administrative Procedures

| | | | | |
|---|---|---|---|---|
| Prior/Concomitant | X | X | X | X |
| Medications^{g} | | | | |
| Compliant with | X | X | X | X |
| GFD: | | | | |
| Yes/No^{h} | | | | |

Clinical Procedures

| | | | | |
|---|---|---|---|---|
| Vital Signs^{j} | X, 4 h | | X | X, 4 h |
| Weight | | | X | |
| Height | | | | |
| Physical | | | | |
| Examination^{k} | | | | |
| 12-lead ECG^{l} | | | | |
| Adverse Event | X | X | X | X |
| Monitoring^{m} | | | | |
| Endoscopy/Duodenal | | | | |
| Biopsy^{a} | | | | |

Clinical Outcome Assessments

| | | | | | | | | | | | | | | | | |
|---|---|---|---|---|---|---|---|---|---|---|---|---|---|---|---|---|
| Provide and/or Collect | | | | | | | | | | | | | | | | |
| ePRO Device | | | | | | | | | | | | | | | | |
| Daily CeD PROⁱ | X | X | X | X | X | X | X | X | X | X | X | X | X | X | X | X |
| BSFS⁺PGA-BFⁱ | X | X | X | X | X | X | X | X | X | X | X | X | X | X | X | X |
| PGA-Sⁱ | X | | | | | | | | | X | | | X | | | |
| CGA^{o} | X | | | | | | | | | X | | | X | | | |
| ICDSQⁱ | X | | | | | | | | | X | | | | X | | |
| SF-12v2ⁱ | X | | | | | | | | | X | | | | X | | |

Laboratory Assessments

| | | |
|---|---|---|
| Hematology/ | X | X |
| Coagulation | | |
| Blood Chemistry 1 | X | X |
| Blood Chemistry 2 | X | X |
| Urinalysis^{p} | X | X |
| Pregnancy | | X |
| Testing^{q} | | |
| Serum Celiac Disease | X | X |
| Serology^{r} | | |

Laboratory Assessments (cont)

| | | |
|---|---|---|
| Exposure | X, 45 m | |
| Pharmacokinetics^{s} | | |
| Serum Anti-Nexvax2 | X | |
| Antibodies | | |
| Serum Cytokines | X, 4 h | X, 4 h |
| (EL-2, IL-8, | | |
| IL-10, and | | |
| CCL20)^{t} | | |

Administration of IP

| | | | | | | | | | | | | | | | |
|---|---|---|---|---|---|---|---|---|---|---|---|---|---|---|---|
| IP Administration by | | | | | | | | | | | | | | | |
| Site Staff | | | | | | | | | | | | | | | |
| Patient Self- | X | X | X | X | X | X | X | X | X | X | X | X | | X | X |
| administration of IP | | | | | | | | | | | | | | | |
| Dispense Pre-filled | X | | | | | | | | | X | | | | | |
| Syringes | | | | | | | | | | | | | | | |
| Return of Pre-filled | | | | | | | | | | | | | X^{w} | | |
| Syringes | | | | | | | | | | | | | | | |

FC Procedure

| | |
|---|---|
| Unmasked Gluten FC^{u} | |
| Masked Gluten/Sham | X |
| FC^{u} | |

| | | | | | | | | | | | | Observationa | |
|---|---|---|---|---|---|---|---|---|---|---|---|---|---|
| | Treatment Period | | | | | | | | | | | 1 Follow-up | |
| | Maintenance Phase (Continued) | | | | | | | | | | | Phase | |
| | | | V34 | | | | | | V40 | | V42^{x} | E | |
| | | | (MF | | | | | | (MF | | (EOT/ | G | V43^{b} |
| Visit | V32 | V33 | C2) | V35 | V36 | V37 | V38 | V39 | C3) | V41 | ET) | D2 | (EOS) |
| Day | 88 | 92 | 93 | 94 | 95 | 99 | 102 | 106 | 107 | 108 | 113 | 120 ± 2 | 141 ± 3 |
| Week | 13 | 14 | 14 | 14 | 14 | 15 | 15 | 16 | 16 | 16 | 17 | 18 | 21^{b} |
| Nexvax2 (Arm A & C) | 900 | 900 | | | 900 | 900 | 900 | 900 | | | 900 | | |
| (μg) | | | | | | | | | | | | | |
| Placebo (Arm B & D) | 0 | 0 | | | 0 | 0 | 0 | 0 | | | 0 | | |
| (μg) | | | | | | | | | | | | | |
| Dose Number | 26 | 27 | | | 28 | 29 | 30 | 31 | | | 32 | | |

Visit Locations

| | | | | | | | | | | | | | |
|---|---|---|---|---|---|---|---|---|---|---|---|---|---|
| Study Site | | | X | | | | | X | X | | X | (X) | X |
| Patient's Home | X | X | | X | X | X | X | | | X | | | |

Administrative Procedures

| | | | | | | |
|---|---|---|---|---|---|---|
| Prior/Concomitant | X | X | X | X | | X |
| Medications^{g} | | | | | | |
| Compliant with GFD: | X | X | X | X | (X) | X |
| Yes/No^{h} | | | | | | |

Clinical Procedures

| | | | | | | |
|---|---|---|---|---|---|---|
| Vital Signs^{j} | X, 4 h | | X, 4 h | X, 4 h | | |
| Weight | | | | X | | |
| Height | | | | | | |
| Physical Examination^{k} | | | | X | | X |
| 12-leadECG^{l} | | | | X | | |
| Adverse Event | X | X | X | X | | X |
| Monitoring^{m} | | | | | | |
| Endoscopy/Duodenal | | | | | (X) | |
| Biopsy^{a} | | | | | | |

Clinical Outcome Assessments

| | | | | | | | | | | | | | |
|---|---|---|---|---|---|---|---|---|---|---|---|---|---|
| Provide and/or Collect | | | | | | | | | | | | | X |
| ePRO Device | | | | | | | | | | | | | |
| Daily CeD PROⁱ | X | X | X | X | X | X | X | X | X | X | X | (X) | X |
| BSFS + PGA-BFⁱ | X | X | X | X | X | X | X | X | X | X | X | (X) | X |
| PGA-Sⁱ | | | X | | | | | | X | | X | | X |
| CGA^{o} | | | X | | | | | | X | | X | | X |
| ICDSQⁱ | | | | X | | | | | | X | | | X |
| SF-12v2ⁱ | | | | X | | | | | | X | | | X |

Laboratory Assessments

| | | |
|---|---|---|
| Hematology/ | X | X |
| Coagulation | | |
| Blood Chemistry 1 | X | X |
| Blood Chemistry 2 | X | X |
| Urinalysis^{p} | X | X |

Laboratory Assessments (cont)

| | | | | | |
|---|---|---|---|---|---|
| Pregnancy Testing^{q} | | | | X | X |
| Serum Celiac Disease | | | | X | X |
| Serology^{r} | | | | | |
| Exposure | | X, 45 m | | X, 45 m | |
| Pharmacokinetics^{s} | | | | | |
| Serum Anti-Nexvax2 | | | | X | X |
| Antibodies | | | | | |
| Serum Cytokines | X, 4 h | X, 4 h | X, 4 h | X, 4 h | |
| (IL-2, IL-8, | | | | | |
| IL-10, and | | | | | |
| CCL20)^{t} | | | | | |

Administration of IP

| | | | | | | | | |
|---|---|---|---|---|---|---|---|---|
| IP Administration by | | | | | | | | |
| Site Staff | | | | | | | | |
| Patient Self- | X | X | | X | X | X | X | X |
| administration of IP | | | | | | | | |
| Return of Pre-filled | | | X^{w} | | | | | X^{w, y} |
| Syringes | | | | | | | | |

FC Procedure

| | | |
|---|---|---|
| Unmasked Gluten FC^{u} | | |
| Masked Gluten/Sham | X | X |
| FC^{u} | | |

AE = adverse event; BSFS + PGA-BF = Bristol Stool Form Scale plus Patient Global Assessment of bowel function; CCL20 = chemokine C-C motif ligand 20; CDAT = Celiac Dietary Adherence Test; CeD = celiac disease; CGA = Clinician Global Assessment; DGP = deamidated gliadin peptide; ECG = electrocardiogram; EGD = esophagogastroduodenoscopy; EOS = End of Study; EOT = End of Treatment; ePRO = electronic patient-reported outcome; ET = Early Termination; FC = food challenge; GFD = gluten-free diet; GLOSS = Global Symptom Survey; h = hour; HLA = human leukocyte antigen; ICDSQ = Impact of Celiac Disease Symptoms Questionnaire; ICF = informed consent form; IgA = immunoglobulin A; IGFD = Impact of a Gluten-free Diet; IgG = immunoglobulin G; IL-2 = interleukin-2; IL-8 = interleukin-8; IE-10 = interleukin-10; IP = investigational product; MFC = masked food challenge; PC = phone call; PGA-S = Patient Global Assessment of symptom severity; PRO = patient-reported outcome; SF-12v2 = 12-item Short Form Health Survey Version 2; SFC = screening food challenge; SQ = subcutaneous; TG2 = transglutaminase 2; V = Visit
Note:
Visit days are ±1 day unless otherwise noted, and the interval between doses when administered 2 times per week can be no more than 6 days (144 hours) and no less than 2 days (48 hours); IP dose frequency is 2 times per week except for the last dose, which is 1 week after the penultimate (i.e., second-to-last) dose. “X” indicates that the assessment/procedure is performed pre-SFC or pre-dose, and “X, #h” indicates that the assessment/procedure is performed pre-FC/pre-dose and also at the number of hours later (#h) thereafter.
^{a}The EGD visits occur at an alternate location if the study site does not have endoscopy capability. In a subset of HLA-DQ2.5 non-homozygous patients, 6 biopsies of the second part of the duodenum are collected at each endoscopy, with 1 pass of the forceps per biopsy. These 6 biopsy samples are used for quantitative histology and stored for exploratory analyses. The second endoscopy can occur 7 ± 2 days after EOT visit. Only those patients having an endoscopy have the assessments in parentheses.
^{b}At the discretion of the investigator with consultation of the Medical Monitor, patients have up to an additional 11 weeks of updosing as unscheduled visits during the treatment period, for a total of approximately 37 weeks of study participation (Screening to Study Completion).
^{c}Before enrollment in the study and any study procedures being performed, all potential patients sign and date an ICF.
^{d}Inclusion and exclusion criteria is assessed at screening (V1) and reassessed at V5 pre-dose to ensure each patient continues to meet all of the inclusion criteria and none of the exclusion criteria prior to treatment with the IP. At V5, the patient must meet additional randomization criteria.
^{e}Each patient's medical and surgical history is completed at screening (V1). Any AEs that occur after ICF signing but before the SFC (unmasked FC containing gluten) is recorded as medical history. Information collected in the “Clinical Characteristics of CeD” survey form is considered the primary source for clinical details regarding CeD.
^{f}Ensure that documents confirming the patient's diagnosis of celiac disease are complete. Sites complete a screening form, which is discussed with the Medical Monitor in uncertain cases for review and approval prior to randomization. Historical documents supporting diagnosis of celiac disease includes histology and serology, and in some cases, genetic tests. An HLA-DQ gene test is performed for all patients at screening and replaces any previous HLA-DQ gene tests results.
^{g}Complete medication history for the 6 months prior to the screening visit (V1) is reported as prior medication. The use of concomitant medications is assessed continuously throughout the study. Medications include all prescription drugs, herbal products, vitamins, minerals, and over-the-counter medications/supplements.
^{h}Patients have initiated a GFD at least 12 months prior to screening. At the screening visit (V1), patients are asked if they adhere to a GFD, and at each subsequent visit, patients is asked if they are aware of consuming gluten-containing food since the previous visit.
ⁱPatient-reported questionnaires are completed on handheld devices at specified timepoints starting at V1. At screening, the modified CeD PRO and GLOSS are completed within 1 hour pre-SFC and again hourly up to 6 hours post-SFC; all have a window of ±10 minutes. The daily CeD PRO is completed every evening at approximately the same time starting from V2. The PGA-S is completed in the evening on the specified days.
^{j}Vital signs include oral body temperature, pulse, blood pressure, and respiratory rate at specified times. Patients are in a semi-supine position. During visits when ECGs are not scheduled, vital sign measurements are taken while patients are in a semi-supine position after a 5-minute rest period. All vital sign assessments have a window of ±15 minutes. Vital sign measurements are taken before the collection of blood samples.
^{k}A complete physical examination is performed at screening (V1), at EOT/ET (V42), and EOS (V43). In addition, at the discretion of the investigator, a targeted or complete physical examination is performed at other visits as deemed necessary.
^{l}The patient is semi-supine for at least 2 minutes before obtaining the ECG, and the ECG is performed before measurement of vital signs and collection of blood samples for laboratory testing. The ECG assessment has a window of ±15 minutes.
^{m}AEs are assessed continuously throughout the study: AEs are solicited at the specified visits, and patients have been encouraged to report AEs at all other times. Any AEs that occur during the 6-hour post-SFC period and the screening period overall will be recorded and graded according to Common Terminology Criteria for Adverse Events, Version 4.03 and analyzed separately from treatment-emergent AEs; they are not considered a part of the medical history.
ⁿAt V5, patients who do not satisfy the inclusion/exclusion criteria for randomization are return their handheld device used during screening.
^{o}The clinician (i.e., Principal Investigator or designee) completes a global assessment of the patient's symptoms at specified visits prior to the patient leaving the site. The CGA at V5 is completed pre-dose and before any clinical procedures or other clinical outcome assessments. For all other visits, the CGA is the last assessment to be completed and is completed at least 4 h after FC at visits that include FC.
^{p}Urinalysis is performed via dipstick, and a microscopic examination is subsequently performed only if needed, depending on the result of the dipstick.
^{q}Urine and serum pregnancy testing (female patients of childbearing potential) are performed at screening (V1), and urine pregnancy tests at the site are performed at V5 prior to randomization and at V28. Urine pregnancy testing are also performed at EOT/ET (V42) and EOS (V43). A positive urine pregnancy test at V1 precludes participation in the SFC (serum results are not yet available).
^{r}Celiac disease-specific serology consists of serum IgA specific for human TG2 and IgG specific for DGP. Total IgA is also measured at V1 only.
^{s}Blood samples for exposure pharmacokinetics are collected within 30 minutes prior to dosing and at 45 minutes (±5 minutes) after administration of IP. Collection is timed from when the needle is withdrawn after SQ injection. Blood samples is collected after ECG and vital signs.
^{t}Pre-dose and pre-FC samples for serum cytokines/chemokines are collected within 30 minutes prior to dosing or the FC. The post-dose and post-FC samples have a window of ±15 minutes.
^{u}Each FC is consumed in the morning on an empty stomach with subjects not having eaten or consumed anything other than clear liquids after midnight before MFC. During the screening period, an unmasked gluten FC is consumed. During the treatment period, a masked gluten FC or sham gluten-free FC is consumed.
^{v}PC indicates visits completed via phone call. Patients are queried about compliance with GFD, AE occurrence and prior/concomitant medication use and also are given the opportunity to ask questions about self-administration of IP.
^{w}Used pre-filled syringes in the provided sharps container ar e returned.
^{x}Early Termination is completed if the patient withdraws from the treatment period prior to EOT (V42).
^{y}Unused pre-filled syringes are returned.

### Screening Period

Patient eligibility for initial enrollment and for randomization to treatment is determined during a screening period of 6 weeks.

On the first day of screening (note: all Visit 1 assessments must occur on a single day), patients who meet initial eligibility criteria, including having a negative urine pregnancy test for female patients of childbearing potential, complete the Clinical Characteristics of CeD survey, Celiac Disease Adherence Test (CDAT), and Impact of a Gluten-free Diet (IGFD) Questionnaire, and then have an unmasked SFC with gluten. Patients are observed for at least 6 hours after SFC. Clinical outcome assessments are collected using a patient-handheld device. Patients score individual symptoms and overall GI symptoms within the previous hour using a modified version of the CeD PRO and the Global Symptom Survey (GLOSS). These assessments are completed within 1 hour before SFC and again hourly up to 6 hours after SFC. In addition, blood samples are collected before and at 2, 4, and 6 hours after the SFC to assess changes in serum cytokines (IL-2, IL-8, IL-10 and CCL20).

Adverse events during the 6-hour post-SFC period and the screening period overall are recorded and graded according to Common Terminology Criteria for Adverse Events (CTCAE), Version 4.03 and analyzed separately.

To be eligible for randomization, patients must show deterioration from baseline (1 hour prior to SFC) demonstrated by an increase of at least 3 in the GLOSS numerical score at any timepoint from 2 hours to 6 hours post-SFC when compared to pre-SFC GLOSS or a GI AE of at least moderate severity on the first day of screening, following SFC.

Patients are screened over 2 visits. In a subset of HLA-DQ2.5 non-homozygous patients randomized to treatment, the first upper GI endoscopy is performed in the second or third week of screening.

### Treatment Period

The 113-day (approximately 16-week) treatment period includes an updosing phase followed by a maintenance phase, which includes 3 MFCs. Most study visits during the treatment period must occur within 1 day of the specified day.

### Updosing Phase

The updosing phase of the treatment period includes 11 study visits.

Dosing with Nexvax2 occurs 2 times per week, with all doses administered SQ by study staff at the study center. Patients receiving active IP (in Arms A and C) are administered escalating dose levels in the order 1, 3, 9, 30, 60, 90, 150, 300, 450, 600, and 750 μg. Equivalent Arms B and D have placebo administered in a way to maintain blinding.

All dose levels in the updosing phase are administered up to a total of 3 times if a patient experiences Nexvax2-related emergent GI symptoms (in particular, nausea, vomiting, abdominal pain, diarrhea) within 24 hours after dose administration, and these symptoms reach a severity of at least Grade 2 according to the CTCAE, Version 4.03, that justify re-administration of the same dose before further dose increase is given. The decision to repeat a dose level in the updosing phase is determined per investigator assessment and in consultation with the Medical Monitor.

Patients are observed at the site for at least 4 hours after the first dose of Nexvax2 and for at least 30 minutes after each subsequent dose in the updosing phase.

### Maintenance Phase (Including 3 Bolus Masked Food Challenges)

The maintenance phase includes 27 visits, of which 7 occur on-site.

The first maintenance dose of 900 μg of Nexvax2 or placebo is self-administered under the supervision of the staff at the study site. Subsequent maintenance doses of 900 μg of Nexvax2 or placebo are self-administered at the patient's home (unsupervised) or at the study site.

When maintenance dosing is at the study site, patients are observed at the site for at least 30 minutes after dosing; patients are observed for at least 4 hours after the first maintenance dose of Nexvax2 (Visit 16), the penultimate dose (Visit 39), and the last dose (Visit 42). Dose frequency is 2 times per week except for the last dose, which is 1 week after the penultimate dose.

The 3 MFCs during the maintenance phase (MFC1, MFC2, and MFC3 in the SoA), each separated by 2 weeks, are given beginning 5 weeks prior to the EOT. The first (MFC1) is in Week 12, the second (MFC2) is in Week 14, and the third FC during the treatment period (MFC3) is in Week 16. While otherwise remaining on a GFD, patients consume a drink of water mixed with food flavoring and vital wheat gluten (containing approximately 6 g gluten protein) for at least 1 and no more than 2 MFCs. The matched sham MFC is gluten free. Each patient has 3 MFCs, but the order is masked to both the patient and the site.

No Nexvax2 is administered on the same day as an MFC. Each MFC is consumed in the morning as a single bolus. Patients should not eat or drink anything but clear liquids after midnight before MFC. The patient remains at the study site for observation for at least 4 hours after each MFC.

All patients continue to receive blinded Nexvax2 at the maintenance dose of 900 μg (or placebo) 2 times per week during the maintenance phase up to the penultimate IP administration; the last dose of IP is administered 1 week after the penultimate dose.

### Observational Follow-Up Period

All patients who receive Nexva2 (including those who discontinue prematurely for any reason) are followed for 30 days after the last dose of Nexvax2 via 1 on-site study visit.

In the subset of non-homozygote patients who had upper GI endoscopy in the screening period, there is an additional on-site visit at 7 days±2 days after the EOT visit at which the second upper GI endoscopy is performed.

### Randomization and Registration

Central randomization is used to avoid bias in the assignment of patients to double-blind treatment (Nexvax2 or placebo) and to increase the likelihood that known and unknown patient characteristics are evenly distributed across the treatment arms.

Randomization to both the treatment arms (Nexvax2 or placebo) and the MFC sequences (with and without gluten) is double-blind and stratified by HLA-DQ2.5 homozygous/non-homozygous. Within the HLA-DQ2.5 non-homozygote cohort, patient randomization is further stratified based on whether or not they choose to participate in the endoscopy subset, in order to ensure that arms are balanced both in the endoscopy subset as well as among those not participating in the endoscopy research.

This study includes Arms A and C (Nexvax2 900 μg) and Arms B and D (placebo). For the HLA-DQ2.5 non-homozygous patients, the randomization ratio of Arms A:B is 1:1 (note: stratification based on whether or not they choose to participate in the endoscopy subset). For the HLA-DQ2.5 non-homozygous patients, the randomization ratio of arms C:D is 2:1. Patients within each arm are also assigned a sequence for consuming MFCs containing gluten or matched sham; a given sequence may include either 1 or 2 MFCs contain gluten.

### Selection of the Study Population

The population that proceeds to the gluten FC on the first day of screening includes male and female patients 18 to 70 years of age (inclusive) at the time of consent who have a diagnosis of CeD and initiated a GFD at least 12 months prior to screening.

The population that is randomized to treatment and MFCs (including the subset of patients who have upper GI endoscopies) includes the patients described above who, in addition, have historically documented evidence of villous atrophy and CeD-specific serological abnormalities when CeD was diagnosed and are positive for HLA-DQ2.5. In addition, patients also have shown deterioration in GI symptom assessment after the SFC (an unmasked FC containing gluten on the first day of screening).

### Inclusion Criteria for Enrollment

Patients must meet all of the following criteria at screening to be eligible for study participation:
1. Adults 18 to 70 years of age (inclusive) who have signed an informed consent form (ICF).
2. History of medically diagnosed CeD that included assessment of duodenal biopsies.
3. Initiated GFD at least 12 months prior to screening.
4. No known allergy or hypersensitivity to any ingredients, except gluten, in the products used for the FCs (i.e., potato protein, rice starch, guar gum, and fruit drink flavoring [i.e., beet juice, elderberry juice, crystallized lime, and stevia]).
5. Willingness to consume food containing up to 6 g of gluten protein at one time and up to 18 g of gluten protein in total during the study (including screening).
6. Willingness to undergo study procedures, including 2 upper GI endoscopies with duodenal biopsies in a subset of patients. (Final eligibility for the endoscopy subset is dependent on HLA-DQ2.5 non-homozygous status.)
7. Able to read and understand English.
   Exclusion Criteria for Enrollment Patients who meet any of the following criteria at screening are not eligible for study participation:
1. Refractory CeD according to “The Oslo definitions for coeliac disease and related terms” (i.e., persistent or recurrent malabsorptive symptoms and signs with villous atrophy despite a strict GFD for more than 12 months).
2. History of inflammatory bowel disease and/or microscopic colitis.
3. Any medical condition that in the opinion of the investigator may interfere with study conduct.
4. Any medical condition that in the opinion of the investigator would impact the immune response (other than CeD), confound interpretation of study results, or pose an increased risk to the patient.
5. Unable or unwilling to perform self-administration of investigational product (IP).
6. Use of immunomodulatory or immune-suppressing medical treatment during the 6 months prior to the first day of screening (e.g., azathioprine, methotrexate, or biological).
7. Use of oral or parenteral immunomodulatory corticosteroids, including budesonide, within the 6 weeks prior to the first day of screening. Topical or inhaled corticosteroids are acceptable.
8. Dosing with placebo or active IP in a clinical study with Nexvax2.
9. Receipt of any investigational drug in another clinical study within 6 months prior to the first day of screening.
10. Females who are lactating or pregnant, including those with positive urinary pregnancy test on the first day of screening.

### Additional Criteria for Randomization to Treatment

### Inclusion Criteria

1. A history of CeD diagnosed on the basis of duodenal biopsy showing villous atrophy and abnormal CeD-specific serology (e.g., anti-TG2 IgA).
2. Positive for the HLA-DQ2.5 genotype. (Note: only patients with two copies of both the HLA-DQA1*05 and HLA-DQB1*02 alleles are considered homozygotes. Randomization into the corresponding HLA-DQ2.5 non-homozygous and homozygous cohort is tracked centrally and capped.)
3. An increase of at least 3 in the GLOSS numerical score at any timepoint from 2 hours to 6 hours post-SFC when compared to pre-SFC GLOSS or a GI adverse event (AE) of at least moderate severity on the first day of screening after SFC.

### Exclusion Criteria

1. Receipt of any vaccine (e.g., influenza) within 1 week prior to the planned first day of the treatment period.
2. Presence of 1 or more of the following laboratory abnormalities at screening: ALT, AST, alkaline phosphatase, or gamma-glutamyltransferase >2.0×ULN; total bilirubin >2.0×ULN or direct bilirubin >1.0×ULN; serum creatinine >1.5×ULN; hemoglobin levels <10 g/dL; platelet count <75×10⁹/L; neutrophil count <1.5×10⁹/L (i.e., <1500/mm³).
3. Thyroid-stimulating hormone outside the normal range and judged clinically significant by the investigator.
4. White blood cell count outside the normal range and judged clinically significant by the investigator.

### Identity of Investigational Products

Nexvax2 is a 1:1:1 equimolar mixture of 3 active pharmaceutical ingredient peptides dissolved in 0.9% sodium chloride United States Pharmacopeia (USP). The constituent synthetic peptides of Nexvax2 are summarized in Table 5.

TABLE 5
Nexvax2 Constituent Peptides

| | Length | Solubility in | Concentration | |
|---|---|---|---|---|
| | (amino | Normal Saline at | for Final Use | |
| Peptide | acids) | pH 7 (mg/mL) | (mg/mL) | Manufacturer |
| NPL001 | 16 | >50 | 0.5 | C S Bio |
| | | | | (Menlo Park, CA) |
| NPL002 | 15 | ≤25 | 0.5 | C S Bio |
| | | | | (Menlo Park, CA) |
| NPL003 | 16 | >50 | 0.5 | C S Bio |
| | | | | (Menlo Park, CA) |

During the updosing phase, Nexvax2 Sterile Solution for Injection 1.5 mg/mL in vials are used for administration for all updosing levels. Dedicated diluent bottles containing defined volumes of 0.9% sodium chloride USP are provided to prepare suitable concentrations of IP for escalating dose levels during updosing. During the maintenance phase, Nexvax2 Sterile Solution for Injection 1.5 mg/mL in pre-filled BD Neopak™ syringes encased in BD Physioject™ disposable auto-injector are used for administration. The active IP and analogous placebo products are summarized in Table 6. IP vials and auto-injectors are provided to sites in a double-blinded manner.

TABLE 6
Investigational Products

| | | | Route of | Fill | |
|---|---|---|---|---|---|
| Product | Role | Strength | Administration | Volume | Manufacturer |
| Nexvax2 | Active | 1.5 mg/mL in | SQ | 1.3 mL | GRAM |
| Vials | IP | 0.9% NaCl | | | (Grand Rapids, MI) |
| | | USP | | | |
| Nexvax2 | Active | 1.5 mg/mL in | SQ | 0.6 mL | GRAM |
| Pre-filled | IP | 0.9% NaCl | | | (Grand Rapids, MI) |
| Auto- | | USP | | | |
| injectors | | | | | |
| Placebo | Placebo | 0.9% NaCl | SQ | 1.3 mL | GRAM |
| Vials | | USP | | | (Grand Rapids, MI) |
| Placebo | Placebo | 0.9% NaCl | SQ | 0.6 mL | GRAM |
| Pre-filled | | USP | | | (Grand Rapids, MI) |
| Auto- | | | | | |
| injectors | | | | | |

IP = investigational product; GRAM = Grand River Aseptic Manufacturing; NaCl = sodium chloride; SQ = subcutaneous; USP = United States Pharmacopeia.

### Treatment Arms and Regimens

Overall treatment regimens for the 4 treatment arms are summarized in Table 7. Additional details are provided in Section 0.

TABLE 7
Treatment Arms

| Treatment | | |
|---|---|---|
| Arm | Description | Assigned Treatment Regimen |
| A | Nexvax2 | Nexvax2 SQ |
| | | 2 times per week up to the beginning of Week 16 and |
| | | 1 dose in Week 17 |
| | | 1 to 750 μg during updosing phase |
| | | 900 μg during maintenance phase |
| B | Placebo | Placebo SQ |
| | | 2 times per week up to the beginning of Week 16 and |
| | | 1 dose in Week 17 |
| C | Nexvax2 | Nexvax2 SQ |
| | | 2 times per week up to the beginning of Week 16 and |
| | | 1 dose in Week 17 |
| | | 1 to 750 μg during updosing phase |
| | | 900 μg during maintenance phase |
| D | Placebo | Placebo SQ |
| | | 2 times per week up to the beginning of Week 16 and |
| | | 1 dose in Week 17 |

SQ = subcutaneous

### Dosing Schedule

Patients in both treatment arms undergo the same dosing schedule. For detailed timing according to visit days, refer to Table 4.

No IP is administered on the same day that an MFC is given. The unmasked SFC with gluten is at Visit 1. The MFCs are at Week 12, Week 14, and Week 16.

### Treatment Period: Updosing Phase

During the updosing phase, patients are administered IP SQ 2 times per week: on Day 1, then 3 days later, then 4 days later, and alternating every 3 and every 4 days thereafter. Visit/administration windows are ±1 day, and the interval between doses can be no more than 6 days (144 hours) and no less than 2 days (48 hours).

Active IP is administered in 11 stepwise doses of 1, 3, 9, 30, 60, 90, 150, 300, 450, 600, and 750 μg during the updosing phase. IP is administered in a way to maintain blinding between Arm A (active) and Arm B (placebo).

Each dose level may be administered up to a total of 3 times if a patient experiences symptoms that justify re-administration of the same dose before further dose increase is given, per investigator assessment and in consultation with the Medical Monitor.

During the updosing phase, IP is administered both diluted and undiluted from the blinded IP vials, and the injection volume is variable.

### Treatment Period: Maintenance Phase

During the maintenance phase, Nexvax2 900 μs (Arms A and C) or placebo (Arms B and D) is self-administered 2 times per week in an alternating every 3 and every 4 days pattern up to the beginning of Week 16 (as specified in Table 4), during which the interval between doses can be no more than 6 days (144 hours) and no less than 2 days (48 hours). The final dose is 1 week after the penultimate dose (Visit 39). Visit/administration windows are ±1 day.

The IP maintenance dose is administered via an auto-injector with a fill volume of 0.6 mL.

### Investigational Product Management

### Preparation and Dispensing of Investigational Product

Blinded IP is dispensed by the study site according to the randomized treatment assignment.

All IP (vials and auto-injectors) should be brought to ambient temperature prior to administration but should not remain at ambient temperature longer than 2 hours. IP is administered 2 times per week during the entire updosing phase and during the maintenance phase up to the penultimate IP administration; the last dose of IP is administered 1 week after the penultimate dose.

IP is prepared from IP vials as a dilution or remains undiluted; the injection volume varies from 0.1 to 0.9 mL. For the first 6 dose levels (corresponding to Nexvax2 doses of 1, 3, 9, 30, 60, and 90 μg), IP dilutions in 0.9% sodium chloride USP are used. For the next 5 dose levels (corresponding to Nexvax2 doses of 150, 300, 450, 600, and 750 μg), IP is drawn directly, without dilution. Each dose level (1 to 750 μg) is administered once but may be repeated according to the guidelines provided herein.

For SQ injections during the updosing phase, the needle is inserted perpendicular to a gently-pinched skinfold, and once the needle is all the way in, the full dose volume is injected before withdrawing the needle. Administrations alternate by visit between the right and left sides of the abdomen. IP is administered by the staff at the study site during the updosing phase according to the dosing schedule provided herein.

During the maintenance phase, IP in pre-filled auto-injectors is self-administered. For SQ injections during the maintenance phase, the disposable auto-injector is held firmly and pushed down perpendicular to a gently-pinched skinfold. Once the injector button is pressed, the enclosed syringe is held against the skin until the full dose volume has been injected before withdrawing the needle. Administrations alternate by visit between the right and left sides of the abdomen.

Patients are observed at the site for at least 4 hours after the first dose of IP in the updosing phase, for at least 30 minutes after each subsequent dose in the updosing phase, and for at least 30 minutes after each maintenance dose administered at the study site. Patients are also observed for at least 4 hours after the first, penultimate, and last maintenance doses of IP.

### Supply, Storage, and Handling of Investigational Product

IP vials are used during the updosing phase. Nexvax2 (active IP) vials have a total concentration of 1.5 mg/mL and comprise approximately 0.5 mg/mL of each peptide in 0.9% sodium chloride USP packaged in sterile-filled 2-mL amber type 1 glass vials with a fill volume of 1.3 mL. Placebo IP vials comprise 0.9% sodium chloride USP and are packaged similarly to the active IP vials.

Auto-injectors are used during the maintenance phase. Nexvax2 (active IP) auto-injectors have a total concentration of 1.5 mg/mL and comprise approximately 0.5 mg/mL of each peptide in 0.9% sodium chloride packaged in the encased 1-mL syringe with a 0.6 mL fill volume. Placebo IP auto-injectors are packaged similarly to the active IP auto-injectors.

### Storage and Handling

All IP (active and placebo) is stored refrigerated.

IP vials are shipped refrigerated and stored refrigerated at 2° C. to 8° C. (approximately 36° F. to 46° F.) on site. After being prepared in syringes for injection. IP can be stored refrigerated for up to 3 hours. The IP should be brought to ambient temperature prior to administration but should not remain at ambient temperature longer than 2 hours. If there is any delay in dosing beyond 2 hours, the IP should be returned to refrigeration.

IP pre-filled auto-injectors are stored at 2° C. to 8° C. (approximately 36° F. to 46° F.; for a maximum of 24 months) and should be at ambient temperature prior to use. The IP should be at ambient temperature for no more than 2 hours. If there is any delay in dosing beyond 2 hours, the IP should be returned to refrigeration.

### Study Assessments

Table 4 contains the for the timing of all assessments.

When scheduled at the same timepoint, the order of procedures should be as follows: first ECG, then vital signs, and lastly collection of blood samples.

### Efficacy and Pharmacodynamic Assessments

Individual assessments are described below.

### Daily Celiac Disease Patient Reported Outcome (CED PRO)

Patients complete the CeD PRO, a patient-reported outcome instrument developed to assess symptom severity in clinical studies in patients with CeD (Leffler et al. Larazotide acetate for persistent symptoms of celiac disease despite a gluten-free diet: a randomized controlled trial. Gastroenterology 148, 1311-9 (2015)).

The CeD PRO was developed in accordance with the US FDA's guidance for industry on PROs to support labelling claims (2009). The CeD PRO was developed as a daily diary to be self-administered on a hand held, ePRO device, which should take less than 5 minutes to complete each day. It includes 9 items designed to assess a patient's impression of their CeD symptom severity in the past 1 day for the following symptoms: abdominal cramping, abdominal pain, bloating, gas, diarrhea, loose stool, nausea, tiredness, and headaches. Responses are scored on an 11-point Likert scale (range: 0 to 10), from “not experiencing the symptom” to “having the worst possible experience” with higher scores indicating greater symptom severity. The CeD PRO is completed daily every evening (starting from V2) at approximately the same time.

The CeD PRO includes 5 domain scores: the Abdominal Symptoms domain (mean of abdominal cramping, abdominal pain, bloating, and gas items). Diarrhea and Loose stools domain (mean of diarrhea and loose stool items), Nausea domain (nausea item), Total GI domain (mean of the Abdominal Symptoms, Diarrhea and Loose Stool and Nausea domains), and Total non-GI domain score (mean of headache and tiredness items). All domains have a 0 to 10 score.

The CeD PRO was developed based on weekly scoring (i.e., calculating daily scores for each item and creating weekly means based on the number of days data is available [minimum 4 of 7 days], then calculating weekly domain scores as the mean of all relevant items) (Leffler et al. 2015). Bolus ingestion of gluten, post-baseline scoring is based on data from the day of the first MFC with gluten. Similarly, an exploratory efficacy endpoint is based on data from the day of a second MFC with gluten.

### Other Clinical Outcome Measures

Additional clinical outcome assessments during screening and the treatment period include Bristol Stool Form Scale (BSFS)+Patient Global Assessment of bowel function (PGA-BF), Patient Global Assessment of symptom severity (PGA-S), Impact of Celiac Disease Symptoms Questionnaire (ICDSQ), and 12-item Short Form Health Survey Version 2 (SF-12v2); as a complement to the PGA-S, the clinician (Principal Investigator or designee) completes the Clinician Global Assessment (CGA). Besides the CeD PRO, all clinical outcome assessments are exploratory measures.

### Bristol Stool Form Scale (BSFS)

The BSFS is a 7-point pictorial scale for assessment and consistent description of daily stool quality. The BSFS is presented when patients report a bowel movement within the past 24 hours in response to the core PGA-BF item. Further details on the BSFS, including the complete questionnaire, are provided in the study procedure manual.

### Patient Global Assessments (PGA)

### PGA of Bowel Function (PGA-BF)

The PGA-BF is designed to accompany the daily use of the pictorial BSFS. The assessment includes 1 core item with 2 sub-items. The core item asks about the frequency of complete bowel movements in the past 24 hours, with a 0 to 10 response scale. If a bowel movement is reported, patients are then asked to identify which of the BSFS images best describes their typical bowel movement in the past 24 hours (type 1 to type 7). Patients are then asked how many of their bowel movements in the past 24 hours were type 6 or type 7.

### PGA of Symptom Severity (PGA-S)

The PGA-S is completed in the evening on the specified days. The PGA-S is a patient-reported global assessment of symptom severity with a 7 day recall period. Patients are asked to rate the severity of their abdominal pain, abdominal cramps, bloating, gas, nausea, diarrhea, loose stool, overall digestive symptoms, headache, and tiredness.

### Clinician Global Assessments (CGA)

The CGAs are clinician-reported outcomes developed to evaluate the severity (CGA-S) and change (CGA-C) in CeD disease status.

The CGA at V5 is completed pre-dose and before any clinical procedures or other clinical outcome assessments. For all other visits, the CGA is the last assessment to be completed and is completed at least 4 hours after FC at visits that include FC. The CGA-S is a 1 item assessment that asks a clinician to identify the subject's disease activity as complete remission, mild disease, moderate disease, or severe disease using all of the information normally available in their clinical practice. The CGA-C asks the clinician to rate the overall change in the patient in relation to their overall CeD medical history. Ratings range from 1 (“much improved”) to 5 (“much worse”).

### Impact of Celiac Disease Symptoms Questionnaire (ICDSQ)

The ICDSQ evaluates the impact of CeD on health-related quality of life. The ICDSQ has a 7 day recall period and includes 14 items with 4 domains: Daily Activities (4 items), Social Activities (3 items), Emotional Well-being (5 items), and Physical Functioning (2 items). Each item has 5 response options ranging from 0 (“not at all”) to 4 (“completely”). Each domain is scored by computing the mean of the domain items. An overall ICDSQ score is calculated by summing the 4 mean domain scores.

### 12-Item Short Form Health Survey Version 2 (SF-12V2)

The SF-12v2 is a patient-reported generic measure of health status. It consists of 12 items scored as 8 health domains (Physical Functioning, Role Physical, Bodily Pain, General Health, Vitality, Social Functioning, Role Emotional, and Mental Health) and 2 summary scores (Physical and Mental Component Summary scores). A utility score (the SF 6D) can also be estimated based on the SF-12v2. The acute version of the SF-12v2 with a 1 week recall period is used. The SF 12v2 is scored in accordance with the QualityMetric algorithm applied via their computerized scoring software.

### Serum Cytokines/Chemokines

PD is assessed using a systemic marker of T-cell activation (IL-2) and associated markers of immune activation; IL-10 is an anti-inflammatory cytokine released by Tregs and other cells in the innate and adaptive immune systems, while IL 8 and CCL20 are chemokines that recruit and activate immune and inflammatory cells. These cytokines and chemokines were selected because they show elevation between 2 and 6 hours after patients with CeD consume gluten; IL-2 and IL 8 serum levels peak at 4 hours, while IL-10 and CCL20 levels are higher at 6 hours.

Serum cytokine concentration are assessed pre-dose and at 1 or more post-dose timepoints on the same day. Assessments are made at the SFC, at the first dose of IP during the updosing phase, and in the maintenance phase at the first, penultimate, and last doses of IP and at each of the MFCs. The assessment of IL 2 associated with the first MFC containing gluten is a secondary measure/endpoint; the rest are exploratory measures.

Pre-dose and pre-FC samples are collected within 30 minutes prior to dosing or the FC. The post-dose and post-FC samples have a ±15 min window for collection.

### Anti-Drug Antibody Assessment

Serum anti-Nexvax2 anti-drug antibody (ADA) is assessed before the first dose of IP, before the first maintenance dose, at EOT, and at End of Study (EOS).

### Laboratory Clinical Pathology Assessments

Laboratory assessments are outlined in Table 8. All assessments are performed by a central laboratory. Screening samples are obtained under fasting conditions (no food or drink, except water, for at least 8 hours before sample collection).

TABLE 8
Laboratory Assessments

| | |
|---|---|
| Assessment Category | Parameters to be Measured |

Safety Assessments

| Hematology | Hct, Hgb, MCH, MCHC, MCV, platelets, RBC count, RBC morphology, |
|---|---|
| | WBC count (with differential panel: basophils, eosinophils, lymphocytes, |
| | monocytes, neutrophils) |
| Coagulation | PT, PTT |
| Pregnancy | For female patients of childbearing potential (scrum and/or urine test |
| | depending on visit) |
| Chemistry 1 | |
| Electrolytes | Sodium, potassium, chloride, bicarbonate |
| Liver Tests | Total bilirubin, alkaline phosphatase, AST, ALT, GGT; with reflex direct |
| | and indirect bilirubin if total bilirubin is elevated |
| Renal Function | BUN, creatinine |
| Other | Total protein, albumin, calcium, phosphorus, glucose, cholesterol, uric acid, |
| | triglycerides, LDH, magnesium, globulin, creatine kinase |
| Chemistry 2 | TSH |
| Urinalysis | Urinalysis is performed via dipstick and a microscopic exam performed |
| | only if needed, depending on the result of the dipstick. |
| Macroscopic | bilirubin, blood, clarity, color, glucose, ketones, leukocyte esterase, nitrite, |
| | pH, protein, specific gravity, urobilinogen |
| Microscopic | RBC, WBC, casts, crystals, bacteria, epithelial cells, yeast, oval fat bodies |
| Celiac Disease- | |
| related Assessments | Celiac Disease-related Assessments |
| HLA-DQ | HLA-DQA and HLA-DQB alleles assessed to determine presence of HLA- |
| | DQ2.5, and whether alleles apart from HLA-DQA1*05 and HLA-DQBl*02 |
| | are also present. |
| Celiac Disease | TG2-IgA, DGP-IgG and total IgA^{a} |
| Serology | |

1. Ab = antibody; ALT = alanine aminotransferase; AST = aspartate aminotransferase; BUN = blood urea nitrogen; GGT = gamma-glutamyltransferase; HBsAg = hepatitis B antigen; Hct = hematocrit; HCV = hepatitis C virus; Hgb = hemoglobin; HIV = human immunodeficiency virus; IgA = immunoglobulin A; IgG = immunoglobulin G; LDH = lactate dehydrogenase; MCH = mcan corpuscular hemoglobin; MCHC = mcan corpuscular hemoglobin concentration; MCV = mean corpuscular volume; PT = prothrombin time; PTT = partial thromboplastic time; RBC = red blood cell; TSH = thyroid-stimulating hormone; WBC = white blood cell
2. ^{a}Total IgA is measured only at Visit 1.

### Safety Assessments

Safety is assessed through continuous monitoring of AEs and through vital signs, physical examinations, and clinical laboratory evaluations (hematology/coagulation, chemistry [liver tests, electrolytes, renal function tests, and TSH], and urinalysis) at pre-specified timepoints.

Exploratory Assessments from Upper Gastrointestinal Endoscopy/Duodenal Biopsy

In a subset of non-homozygous patients, the effects of Nexvax2 on duodenal histology are compared with placebo treatment. Because individual variation in quantitative measures of duodenal histology can be large, histology assessments are analyzed by treatment group rather than as changes in individual patients.

In a subset of 25 patients per treatment Arms A and B, an upper GT endoscopy and duodenal biopsy for quantitative histology is performed once during the screening period and once during the follow-up period.

Briefly, 6 biopsies of the second part of the duodenum are collected at each endoscopy, with 1 pass of the forceps per biopsy. Samples are stored in fixative, cut, and stained. The 6 samples are used for quantitative histology and stored for exploratory analyses. De-identified histology slides are evaluated by an expert pathology lab for morphometric measurements of villus height and crypt depth and frequency of CD3+ lymphocytes per 100 epithelial cells. Measurements are done by 2 independent pathologists according to previously published protocols.

### Pharmacokinetic Exposure Assessments

Pre-dose and post-dose blood samples for PK assessments of exposure are collected at pre-specified times (within 30 minutes prior to dosing and at 45 minutes after IP administration) on the first day that the maintenance dose is administered and at the penultimate and last IP dose administrations. Blood collection for PK assessments are timed from when the needle is withdrawn after SQ injection. The window for the post-dose sampling is ±5 minutes.

### Example 9. Physical, Chemical, and Pharmaceutical Properties and Formulations of Nexvax2

The 3 peptides in Nexvax2 Sterile Solution for Injection (Nexvax2) correspond to amino acid sequences derived from gluten proteins as provided in Table 9.

TABLE 9
Nexvax2 Peptides

| | | | Molecular | | |
|---|---|---|---|---|---|
| | | Size | Weight | | |
| Identifier | Peptide Sequence | (aa*) | (g/mol) | Protein | Grain |
| NPL001 | (pE)LQPFPQPELPYPQPQ-NH2 | 16 | 1889.3 | α-gliadin | wheat |
| | (SEQ ID NO: 10) | | | | |
| NPL002 | (pE)QPFPQPEQPFPWQP-NH2 | 15 | 1833.2 | ω-gliadin/hordein | wheat/barley |
| | (SEQ ID NO: 11) | | | | |
| NPL003 | (pE)PEQPIPEQPQPUPQQ-NH2 | 16 | 1886.2 | hordein | barley |
| | (SEQ ID NO: 12) | | | | |

*aa = number of amino acids per peptide.

### Physical and Chemical Characteristics

The physical and chemical characteristics of the 3 Nexvax2 peptides are presented in Table 10.

TABLE 10
Physical and Chemical Characteristics of Nexvax2 Peptides

| | Peptide | | |
|---|---|---|---|
| Characteristic | NPL001 | NPL002 | NPL003 |
| Molecular weight | 1889.3 | 1833.2 | 1886.2 |
| (g/mol) | | | |
| Description | White to | White to | White to |
| | off-white powder | off-white powder | off-white powder |
| Solubility in normal | >50 mg/mL | ≤25 mg/mL | >50 mg/mL |
| saline at pH 7 | | | |

### Manufacture of NPL001, NPL002, and NPL003

All peptides in Nexvax2 (NPL001, NPL002, and NPL003) were manufactured in accordance with current Good Manufacturing Practices (cGMPs) at C S Bio (Menlo Park, Calif.).

### Analysis and Characterization of Drug Substance

The identity and purity of the individual Nexvax2 peptides were confirmed by high-performance liquid chromatography (HPLC) and mass spectrometry (MS). Impurities assessed by HPLC assay showed ≤2.0% total impurities of related substances present. The shelf life for each peptide based on stability and use studies was approximately 5 years.

### Drug Product

### Formulation

Nexvax2 Sterile Solution for Injection is manufactured in vials (˜1.5 mg/mL Nexvax2 peptides in 0.9% sodium chloride) for ID and SC administration and prefilled syringes (˜1.5 mg/mL Nexvax2 peptides in 0.9% sodium chloride) for SC administration only, in compliance with cGMPs.

### Manufacture of Nexvax2

Nexvax2 vials and syringes (1 mL Neopak syringes encased in a BD PhysioJect™ Autoinjector for s.c. administration) are manufactured in accordance with the principles of cGMPs at Grand River Aseptic Manufacturing (GRAM; 140 Front Ave, Grand Rapids, Mich. 49504). The manufacturing operations support the preparation of s.c. injections during the updosing and maintenance phases.

### Preparation of Syringes for Updosing of Nexvax2

Investigational product (Nexvax2 1.5 mg/mL) is added to diluent (sodium chloride United States Pharmacopeia [USP] 0.9%) as provided in Table 11 to prepare 10 fixed doses of Nexvax2 for administration as provided in Table 12. Updosing occurs either in a 50 mL vial filled with 44.7 mL sodium chloride USP 0.9% or in a 20 mL vial filled with 14 mL of the same component using Nexvax2 1.5 mg/mL (2 mL amber vial, 1.3 mL fill).

TABLE 11
Dose Presentation, Diluent Bottles, and Concentrations of Nexvax2 After Dilution

| | Sodium Chloride | Volume of Neat | |
|---|---|---|---|
| | USP 0.9% | Investigational Product | Final Nexvax2 |
| | (mL) | Added (mL) | Concentration (mg/mL) |
| Nexvax2 (1.5 mg/mL) | 1.3 | 0 | 1.5 or 0 |
| in 2 mL amber vial | | | |
| Diluent in 20 mL vial | 14 | 1 | 0.1 or 0 |
| Diluent in 50 mL vial | 44.7 | 0.3 | 0.01 or 0 |

USP = United States Pharmacopeia.

TABLE 12
Dose Volumes (mL) for Subcutaneous Updosing Schedule

| | | | Nexvax2 in | Nexvax2 in | Injection |
|---|---|---|---|---|---|
| Dose | Dose Level | Neat Nexvax2 | 20 mL Vial | 50 mL Vial | Volume |
| Number | (Nexvax2 μg) | (1.5 mg/mL) | (0.1 mg/mL) | (0.01 mg/mL) | (mL) |
| Up-dose 1a | 1 | — | — | 0.1 | 0.1 |
| Up-dose 1b | 3 | — | — | 0.3 | 0.3 |
| Up-dose 2 | 9 | — | — | 0.9 | 0.9 |
| Up-dose 3 | 30 | — | 0.3 | — | 0.3 |
| Up-dose 4 | 60 | — | 0.6 | — | 0.6 |
| Up-dose 5 | 90 | — | 0.9 | — | 0.9 |
| Up-dose 6 | 150 | 0.1 | — | — | 0.1 |
| Up-dose 7 | 300 | 0.2 | — | — | 0.2 |
| Up-dose 8 | 450 | 0.3 | — | — | 0.3 |
| Up-dose 9 | 600 | 0.4 | — | — | 0.4 |
| Up-dose 10 | 750 | 0.5 | — | — | 0.5 |

The dose levels of active investigational product (Nexvax2) during updosing begin at 1 or 3 μg, and increase stepwise to 9, 30, 60, 90, 150, 300, 450, 600, and 750 μg before the maintenance doses at 900 Doses during updosing are administered with a single injection in a total volume between 0.3 and 0.9 mL.

### Preparation of Syringes Nested in a BD PhysioJect Autoinjector Device for Maintenance Doses of Nexvax2

The Nexvax2 maintenance dose is the final dosage form (1.5 mg/mL and approximately 0.5 mg/mL of each peptide) provided in Table 10. Nexvax2 is packaged at a 1.5-mg/mL concentration in 0.9% sodium chloride for injection in a 1-mL long Neopak syringe (approximately 0.6 mL fill volume) encased in a BD PhysioJect AutoInjector device.

### Storage and Handling

Nexvax2 vials and Nexvax2 pre-filled syringes are stored refrigerated at 2° C. to 8° C. and should be at ambient temperature (not more than 2 hours) prior to use.

### Example 10. A Phase 1 Study of Nexvax2 Administered Subcutaneously after a Screening Gluten Food Challenge that Compares Relative Bioavailability with Intradermal Administration in Non-Homozygous HLA-DQ2.5+ Adults with Celiac Disease

### Study Rationale

Nexvax2 is planned to be a self-administered maintenance therapy for patients with CeD who carry the HLA-DQ2.5 genotype. The initial indication for Nexvax2 is intended to be protection against symptoms caused by inadvertent gluten exposure in CeD patients following a Gluten-free Diet (GFD).

The purpose of this study is to assess the safety and tolerability of Nexvax2 administered by subcutaneous (SQ) injection during updosing (3 to 750 μg) and at the maintenance dose level of 900 μg, and to compare the relative bioavailability of Nexvax2 peptides following ID and SQ injection of Nexvax2. Twelve patients receive Nexvax2, and 2 patients receive placebo to facilitate a double-blind study design in order to reduce the potential for a nocebo effect. Pharmacokinetic (PK) assessments are performed up to 8 hours after each of 2 SQ and 2 ID administrations of Nexvax2 at the maintenance dose level of 900 μg. Timing of PK assessments are based on previous clinical studies of Nexvax2 administered ID that showed detectable plasma levels from 10 minutes to 2 hours after dosing as well as making the assumption based on PK studies of other small peptides that SQ administration may delay drug absorption and clearance.

This study also assesses evidence of immunological “equivalence” for Nexvax2 administered SQ or ID by measuring the change in serum interleukin (IL)-2 and chemokine C-C motif ligand 20 (CCL20; also called macrophage inflammatory protein-3 alpha [MIP-3a]) concentrations pre-dose to 2, 4, 6 and 8 hours post-dose. Elevations in serum levels of interleukin (IL)-2 and CCL20 are concomitant with the onset of gastrointestinal (GI) symptoms 2-4 hours after administering the first dose of Nexvax2 to CeD patients and also with consumption of a bolus gluten food challenge (hereafter referred to as “FC”) by patients with CeD on GFD.

To focus the clinical development of Nexvax2 on the target population of CeD patients who are most likely to benefit from Nexvax2 treatment, both the present study and a planned phase 2 clinical trial of Nexvax2 each incorporate a single FC on the first day of screening to identify patients who experience GI symptoms after ingesting gluten. Patients who report no overall deterioration in GI symptoms during the 6 hours after the FC do not continue to the treatment phase of the study. However, the FC may affect the tolerability of Nexvax2 because recent ingestion of gluten boosts the immune response to gluten and increases clinical and T-cell responsiveness to Nexvax2 peptides. Hence, the present study provides valuable information regarding the tolerability of Nexvax2 at the low starting dose of 3 μg followed by updosing to the maintenance dose level of 900 μg when the initial updosing phase is preceded by a FC 3-5 weeks earlier. If GI related adverse events are observed following the first dose, the starting dose may be reduced from 3 μg to 1 μg.

### Study Design

This is a Phase 1, randomized, double-blind, placebo-controlled clinical study of Nexvax2, a peptide-based therapeutic vaccine, in adult patients who are non-homozygous for human leukocyte antigen (HLA)-DQ2.5+ with confirmed CeD who, apart from the FC during screening, have been following a GFD for at least 12 consecutive months prior to screening. The study evaluates the safety and tolerability of Nexvax2 administered SQ following a FC and also compares the relative bioavailability of Nexvax2 peptides following maintenance doses of Nexvax2 (900 μg) administered SQ and ID. The pharmacodynamics of Nexvax2, using serum cytokine assessments, are also compared after maintenance doses of Nexvax2 (900 μg) are administered SQ and ID.

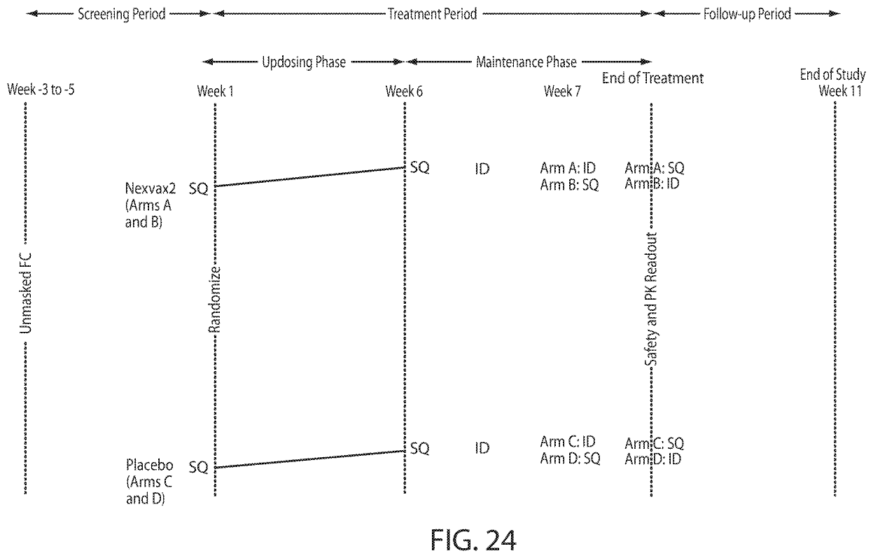

The study design is summarized in FIG. 24.

The study plan consists of 3 phases: a screening period of 3 to 5 weeks, a 46-day treatment period, and a 30-day post-treatment observational follow-up period. The treatment period includes an initial updosing phase.

On the first day of screening, patients who meet initial eligibility criteria are enrolled and have further clinical assessments, blood tests, and then a FC followed by a 6-hour observation period. During the observation period, Patient-Reported Outcomes (PROs) are assessed for changes, and additional blood samples are assessed for elevations in serum cytokine levels. Patients who meet all inclusion and none of the exclusion criteria, including the criteria for randomization, are randomized 6:6:1:1 to Arms A, B, C, or D, respectively, with Arms A and B receiving Nexvax2 and Arms C and D receiving placebo. All Nexvax2 investigational product (IP) is administered 2 times per week.

All patients receiving Nexvax2 have updosing starting from 3 μg with stepwise dose increments before reaching the maintenance dose of 900 μg. If GI related adverse events are observed following the first dose, the starting dose may be reduced from 3 μg to 1 μg. All updosing injections and the first maintenance dose of 900 μg are administered by SQ administration. The second maintenance dose of 900 μg are given by ID administration. Then, to facilitate the comparison of ID and SQ administration, there is a crossover phase with 2 arms: Arm A, which has the ID then SQ dosing order, and Arm B, which has the SQ then ID dosing order. All patients thus receive 4 doses total (with each dose consisting of 6 injections administered within 2 minutes) at the maintenance dose level. Equivalent Arms C and D have placebo administration in the same ID/SQ and SQ/ID order as Arms A and B, respectively. Randomization to Arm A versus C and Arm B versus D is blinded. To meet the primary PK objective, blood samples for serial PK assessments of Nexvax2 exposure is collected pre-dose and at multiple timepoints post-dose (ranging from 10 minutes to 8 hours after administration) on the days that the maintenance dose is administered.

With the exception of protocol-specified gluten consumption at the screening FC, patients continue adhering to their established, pre-enrollment GFD unchanged.

Patients who discontinue treatment prematurely continue study assessments as long as they do not withdraw consent. Up to 4 randomized patients who receive at least 1 dose of IP and then discontinue treatment, in addition to any randomized patients who never received IP, may be replaced in order to maximize the available bioavailability data.

### Screening Period

Patient eligibility for initial enrollment and for randomization to treatment is determined during a screening period of no less than 3 weeks and up to 5 weeks, which includes collection of CeD-specific serology tests, patient-reported compliance with a GFD, and HLA-DQ genotype assessment. At the start of the screening period, patients have an unmasked FC and then are observed for at least 6 hours. PROs relating to symptoms during the previous 1 hour are recorded within 1 hour before FC and again at 2, 3, 4, 5, and 6 hours after FC. Serum cytokines are assessed before FC and at 2, 4, and 6 hours after FC. Adverse events during the 6-hour post-FC period and the screening period overall are recorded and graded according to Common Terminology Criteria for Adverse Events (CTCAE), Version 4.03 and analyzed separately. Patients who report no overall deterioration in GI symptoms during the 6 hours after the FC on screening Day 1 as well as patients who are either not positive for HLA-DQ2.5 or who are homozygous for HLA-DQ2.5 do not continue to the treatment phase of the study.

### Treatment Period (Including Updosing Phase and Maintenance Dose Phase

Dosing with IP occurs 2 times per week, with all doses administered by study staff at the study center. The first 10 doses are SQ in all treatment arms. Patients receiving active IP are administered escalating dose levels of Nexvax2 in the order 3, 9, 30, 60, 90, 150, 300, 450, 600, and 750 μg in Arms A and B, followed by the maintenance doses of 900 μg (Arm A: SQ, ID, ID, and SQ; Arm B: SQ, ID, SQ, and ID). Equivalent Arms C and D have placebo administered by the same routes and syringe types in the same order as Arms A and B, respectively. IP is administered in a way to maintain blinding between Arms A and B versus Arms C and D.

If GI related adverse events are observed following the first dose, the starting dose may be reduced from 3 μg to 1 μg. All dose levels in the updosing phase may be repeated twice (i.e., up to a total of 3 doses per dose level) if a patient experiences IP-related emergent GI symptoms (in particular, nausea, vomiting, abdominal pain, and diarrhea) within 24 hours after dose administration, and these symptoms reach a severity of Grade 2 according to CTCAE, Version 4.03, that justify re-administration of the same dose before further dose increase is given.

Patients are observed at the site for at least 8 hours after the first dose of IP and for at least 30 minutes after each subsequent dose in the updosing phase.

Patients are observed at the site for at least 8 hours after each of the 4 maintenance doses.

### Observational Follow-Up Period

All patients who receive IP (including those who discontinue prematurely for any reason) are followed for 30 days after the last dose of IP via 1 on-site study visit.

### Objectives and Endpoints

Primary objectives are to evaluate the safety and tolerability of Nexvax2 administered SQ after a screening FC and to evaluate the relative bioavailability of the 3 individual constituent peptides of Nexvax2 (NPL001, NPL002, and NPL003) after maintenance doses of Nexvax2 are administered by SQ and ID injections.

Primary endpoints: treatment-emergent adverse events (TEAEs) and clinical laboratory measures during the treatment and post-treatment periods and the ratio between SQ and ID administration for the area under the plasma concentration-time curve from time 0 extrapolated to infinity (AUC_{0-∞}) for the 3 individual constituent peptides of Nexvax2 (NPL001, NPL002, and NPL003).

Secondary objectives are to evaluate the PD of maintenance dose levels of Nexvax2 administered SQ and ID as assessed by a systemic marker of T-cell activation (change from pre-dose in serum IL-2 concentration at 2, 4, 6, and 8 hours post-dose); to compare PK parameters including maximal plasma concentration (Cₘₐₓ), elimination half-life (t_{1/2}), time to maximal plasma concentration (Tₘₐₓ), and area under the plasma concentration-time curve from time 0 extrapolated to 8 hours (AUC_{0-8 h}) for each of the NPL001, NPL002, and NPL003 peptides in Nexvax2 after SQ and ID maintenance doses.

Secondary endpoints: the change in the serum IL-2 concentration at 2, 4, 6 and 8 hours post-dose from within 30 minutes pre-dose after the first maintenance dose of Nexvax2 (900 μg), which is administered SQ, and the second maintenance dose of Nexvax2, which is administered ID; the 2, 4, 6 and 8-hour change in serum IL-2 concentration after the third and fourth administered maintenance doses of Nexvax2 (900 μg); the ratio between SQ and ID administration for individual plasma AUC₀₋₈ h for each of the 3 constituent peptides of Nexvax2 (NPL001, NPL002, and NPL003); the ratio between SQ and ID administration for individual t_{1/2} for each of the 3 constituent peptides of Nexvax2 (NPL001, NPL002, and NPL003); the ratio between SQ and ID administration for individual Cₘₐₓ for each of the 3 constituent peptides of Nexvax2 (NPL001, NPL002, and NPL003); the ratio between SQ and ID administration for individual Tₘₐₓ for each of the 3 constituent peptides of Nexvax2 (NPL001, NPL002, and NPL003).

Exploratory objectives: to evaluate the relative average bioavailability of maintenance dose levels of Nexvax2 administered by SQ and ID injections as assessed by the sum of the plasma concentrations of NPL001, NPL002, and NPL003 peptides; to evaluate the relative bioavailability to the relative bioactivity of Nexvax2 administered SQ or ID after the 3^{rd} and 4^{th} maintenance injections; to evaluate serum levels of anti-Nexvax2 immunoglobulin and their relationship to the plasma AUC_{0-8 h} and AUC_{0-∞} for NPL001, NPL002, and NPL003; to evaluate the elevations in scrum IL-2 and CCL20 after the first (3 μg, or if revised downwards, then 1 μg) and maintenance (900 μg) doses of IP and their relationship to elevations in serum IL-2 and CCL20 after the FC; to evaluate the IL-2 and CCL20 profile 2, 4, 6 and 8 hours after each maintenance dose vs after the initial dose (3 μg, or if revised downwards, then 1 μg); to evaluate the relationship between elevations in serum IL-2 and CCL20, and GI symptoms up to 6 hours after the FC; to assess the occurrence of AEs and onset of GI symptoms up to 6 hours after gluten FC and their relationship to other clinical features.

Exploratory endpoints: the ratio between SQ and ID administration for the sum of the AUC_{0-8 h} for the 3 constituent peptides of Nexvax2 (NPL001, NPL002, and NPL003) after SQ compared to ID (SQ:ID AUC_{0-∞}); ratio of the sum of the AUC_{0-8 h} for the 3 constituent peptides of Nexvax2 (NPL001, NPL002, and NPL003) after SQ to ID (SQ:ID AUC_{0-∞}) compared with the ratio of the change in IL-2 after SQ to ID for the 3^{rd} and 4^{th} maintenance doses of Nexvax2; relationship between levels of anti-drug antibodies (ADAs) before the first dose of IP in the maintenance phase and the sum of the plasma AUC_{0-8 h} and of the plasma AUC_{0-∞} for the 3 constituent peptides of Nexvax2 (NPL001, NPL002, and NPL003) after the first dose of IP in the maintenance phase; change in serum IL-2 and CCL20 concentrations after the FC and after the first and maintenance administrations of IP; change in serum IL-2 and CCL20 concentrations after the first compared to maintenance administrations of IP; relationship between change in serum IL-2 and CCL20 concentrations and changes in PROs up to 6 hours after the FC; changes in PROs and AE profile up to 6 hours after the FC; relationship between changes in PROs and AEs up to 6 hours after the FC and reason for suspicion of CeD at diagnosis; relationship between changes in PROs and AEs up to 6 hours after the FC and patient-reported history of symptoms after gluten exposure in the past.

### Treatment Arms

This study includes the following four treatment arms.

| | | Maintenance Dose |
|---|---|---|
| Arm | IP Administered | Administered |
| A | Nexvax2 900 μg | SQ, ID, ID, SQ |
| B | Nexvax2 900 μg | SQ, ID, SQ, ID |
| C | Placebo | SQ, ID, ID, SQ |
| D | Placebo | SQ, ID, SQ, ID |

ID = intradermal; IP = investigational product; SQ = subcutaneous. Arms A, B, C, and D are randomized 6:6:1:1.

### Duration of Study Participation

The total duration of study participation is up to approximately 16 weeks, including the up to 35-day (3- to 5-week) screening period, 46-day (approximately 7-week) treatment period, and 30-day (approximately 4-week) observational follow-up period. Patients may have up to an additional 10 weeks of updosing as unscheduled visits during the treatment period, for a total of 26 weeks of study participation.

### Inclusion and Exclusion Criteria

### Inclusion Criteria for Enrollment:

1. Adults 18 to 70 years of age (inclusive) who have signed an informed consent form (ICF).
2. History of medically diagnosed CeD that included assessment of duodenal biopsies.
3. Maintenance of GFD for at least 12 consecutive months prior to screening.
4. Willingness to consume a moderate amount of gluten equivalent to approximately that in 2 slices of wheat bread at one time during screening.
5. Able to read and understand English.

### Exclusion Criteria for Enrollment

1. Refractory CeD according to “The Oslo definitions for coeliac disease and related terms” (i.e., persistent or recurrent malabsorptive symptoms and signs with villous atrophy despite a strict GFD for more than 12 months).
2. History of inflammatory bowel disease and/or microscopic colitis.
3. Any medical condition that in the opinion of the investigator may interfere with study conduct.
4. Any medical condition that in the opinion of the investigator would impact the immune response (other than CeD), confound interpretation of study results, or pose an increased risk to the patient.
5. Use of immunomodulatory or immune-suppressing medical treatment during the 6 months prior to the first day of screening (e.g., azathioprine, methotrexate, or biological).
6. Use of oral or parenteral immunomodulatory corticosteroids, including budesonide, within the 6 weeks prior to the first day of screening. Topical or inhaled corticosteroids are acceptable.
7. Dosing with placebo or active IP in a clinical study with Nexvax2.
8. Receipt of any investigational drug or participation in another clinical study within 6 months prior to the first day of screening.
9. Females who are lactating or pregnant, including those with positive urinary pregnancy test on the first day of screening.

### Additional Criteria for Randomization to Treatment

### Inclusion Criteria

1. A history of CeD diagnosed on the basis of duodenal biopsy showing villous atrophy and abnormal CeD-specific serology (e.g., anti-transglutaminase 2 [TG2] IgA).
2. Positive for the HLA DQ2.5 genotype, which is encoded by HLA-DQA1*05 (or other alleles prefixed with “HLA-DQA1*05” such as HLA-DQA1*0501) and HLA-DQB1*02 (or other alleles prefixed with “HLA-DQB1*02” such as HLA-DQB1*0201).

### Exclusion Criteria

1. No overall deterioration from baseline (1 hour prior to FC) in the average of Global Symptom Survey (GLOSS) scores at 2, 3, 4, 5 and 6 hours after the FC on screening Day 1.
2. Receipt of any vaccine (e.g., influenza) within 1 week prior to planned first day of the treatment period.
3. Homozygous for HLA-DQ2.5, as confirmed by the absence of HLA-DQA1 alleles in addition to HLA-DQA1*05 (or others prefixed with “HLA-DQA1*05”) and the absence of HLA-DQB1 alleles in addition to HLA-DQB1*02 (or others prefixed with HLA-DQB1*02″).
4. Presence of 1 or more of the following laboratory abnormalities at screening:
   a. alanine aminotransferase, aspartate aminotransferase, alkaline phosphatase, or gamma-glutamyltransferase >2× the upper limit of normal (ULN).
   b. total bilirubin >2.0×ULN or direct bilirubin >1.0×ULN.
   c. serum creatinine >1.5×ULN.
   d. hemoglobin levels <10 g/dL.
   e. platelet count <75×10⁹/L.
   f. Thyroid-stimulating hormone outside the normal range and judged clinically significant by the investigator.
   g. Neutrophil count <1.5×10⁹/L (i.e., <1500/mm³).
   h. White blood cell count outside the normal range and judged clinically significant by the investigator.

### IP, Dosage, and Route of Administration

The active IP consists of Nexvax2 Sterile Solution for Injection 1.5 mg/mL in vials. Nexvax2 is a 1:1:1 equimolar mixture of 3 active pharmaceutical ingredient peptides (NPL001, NPL002, and NPL003) dissolved in 0.9% sodium chloride United States Pharmacopeia (USP). Matching placebo consists of 0.9% sodium chloride USP.

During the updosing phase, IP is administered both diluted and undiluted from the IP vials, and the injection volume varies from 0.1 to 0.9 mL. During the updosing phase, IP is administered from 1 mL or 3 mL plastic syringes fitted with a 30G×½ inch needle. For the first 5 dose levels (corresponding to Nexvax2 doses of 3, 9, 30, 60, and 90 μg), IP dilutions in 0.9% sodium chloride USP are used. For the next 5 dose levels (corresponding to Nexvax2 doses of 150, 300, 450, 600, and 750 μs), IP is drawn directly, without dilution. IP is administered 2 times per week SQ by the study staff. Each dose level (3 to 750 μg) is administered once but may be repeated according to the guidelines in Study Design (see above). If GI related adverse events are observed following the first dose, the starting dose may be reduced from 3 μg to 1 μg.

During the maintenance phase, undiluted IP from vials is drawn into and administered from six 1-mL syringes fitted with detachable 30G needles that are 1.5 mm in length for ID injections or ½ inch (13 mm) in length for SQ injections. The total injection volume is 0.6 mL administered in 6 divided doses of 0.1 mL as separate injections, administered within 2 minutes.

Site staff perform all injections. For both ID and SQ injections, the needle is inserted perpendicular to a gently-pinched skinfold, and once the needle is all the way in, the full dose volume is injected before withdrawing the needle. Administrations alternate by visit between the right and left sides of the abdomen. Skin bleb formation and any immediate leakage from the injection site are recorded. IP is administered 2 times per week during both the maintenance phase and the updosing phase.

The placebo vials and syringes are identical to the active IP vials and syringes except for the lack of active ingredient.

### PD Assessments

PD is assessed using serum markers of immune activation (IL-2 and CCL20). Changes in serum biomarkers are expressed as change from pre-dose levels on the same day. Assessments are made before and 2, 4, and 6 hours after the screening FC; before and 2, 4, 6 and 8 hours after the first dose of IP during the updosing phase; and before and 2, 4, 6 and 8 hours after each dose of IP administered SQ and ID in the maintenance phase.

### Safety Assessments

Safety is assessed through continuous monitoring of AEs (investigators assess AEs in relation to treatment and to potential gluten exposure) and through vital signs, physical examinations, clinical laboratory evaluations (hematology/coagulation, chemistry [liver tests, electrolytes, and renal function tests], and urinalysis), and CeD-specific serology at pre-specified timepoints. Both treatment-emergent AEs and AEs during the screening period, including the 6 hours after the FC, are assessed.

### PRO Assessments

A modified Celiac Disease Patient Reported Outcome (CeD PRO®) questionnaire and the GLOSS are used to assess symptoms during the previous 1 hour at the following timepoints: within 1 hour before FC and again at 2, 3, 4, 5, and 6 hours after the FC.

### ADA Assessments

Serum anti-Nexvax2 antibody (ADA) is assessed before the first dose of IP, before the first maintenance dose, and at End of Study (EOS). Elevated levels of ADAs are investigated by assessments of immunoglobulin levels specific for NPL001, NPL002, and NPL003.

### PK Assessments

Pre-dose and post-dose blood samples for PK assessments of exposure and bioavailability are collected at pre-specified times (within 30 minutes prior to IP administration; 10, 20, 30, and 45 minutes after IP administration; and 1, 1.5, 2, 3, 4, 5, 6, and 8 hours after IP administration) on the days that the maintenance dose is administered. Blood collection for PK assessments is timed from when the needle is withdrawn after SQ injection or, for ID injections, after the sixth (i.e., final) injection.

### Statistical Methods

Analysis populations are as follows. The Intent-to-treat (ITT) Population consists of all randomized patients who received at least 1 dose of IP. The PK Population consists of all patients in the ITT Population who have PK assessments from pre-dose plasma samples and from at least 10 post-dose plasma samples obtained up to 8 hours post-dose without missing 2 consecutive planned collections after at least 1 SQ and 1 ID administration of Nexvax2 at the maintenance dose. The Per-Protocol Population consists of all patients in the ITT Population who completed the study through the End of Treatment visit with no major protocol violations. The Safety Population is identical to the ITT Population. The Gluten Food Challenge Population consists of all patients who received the FC on the first day of screening.

PK analyses are based on the PK Population. Relative bioavailability of the SQ and ID injections with respect to plasma AUC_{0-∞} for the 3 individual constituent peptides of Nexvax2 are established based on the 12 patients randomized to the Nexvax2 treatment group.

### Safety Analysis

AEs are collected from the time patients sign the ICF. TEAEs, vital sign measurements, and clinical laboratory information is tabulated and summarized by treatment group and treatment aim. All TEAEs are summarized by system organ class, preferred term, severity (grades as defined in CTCAE, Version 4.03), and relationship to IP. Proportions of patients in each treatment group who experience new major organ manifestations during the study are summarized.

AEs during the screening period, including the 6 hours after FC on the first day of screening, are separately tabulated and summarized for all patients who received the FC, whether or not they continue in the study. All screening AEs are summarized by system organ class, preferred term, severity (grades as defined in CTCAE, Version 4.03), and relationship to FC.

### Sample Size

A total of 14 patients are randomized. Approximately 40 patients are screened. Patients are randomized in a 6:1 ratio to the Nexvax2:placebo treatment groups. Within each treatment group, patients are randomized in a 1:1 ratio to each of the arms (i.e., 6 patients each in [active IP] Arms A and B and 1 patient each in [placebo] Arms C and D). Based on other studies, the coefficient of variation (CV) is assumed to be between 18.8-24.0 for the 3 constituent peptides (NPL001, NPL002, and NPL003). Based on these CV estimates, a sample size of 12 patients yields approximately 80-95% power for the AUC_{0-∞} relative bioavailability analyses.

Up to 4 randomized patients who receive at least 1 dose of IP and then discontinue treatment, in addition to any randomized patients who never received IP, may be replaced. A replacement patient is placed into the same treatment group and treatment arm as the patient being replaced.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method for treating Celiac disease in a subject, the method comprising:
administering to the subject a dose escalation regimen of a gluten peptide composition comprising a first, second and third peptide, wherein the dose escalation regimen comprises administering the following doses sequentially and at least one day apart from each other: 1, 3, 9, 30, 60, 90, 150, 300, 450, 600 and 750 micrograms of the gluten peptide composition; and
subsequently administering to the subject during a tolerizing regimen a dose of 900 micrograms of the gluten peptide composition,
wherein:
the first peptide comprises the amino acid sequence ELQPFPQPELPYPQPQ (SEQ ID NO: 1), wherein the N-terminal glutamate is a pyroglutamate and the C-terminal glutamine is amidated;
the second peptide comprises the amino acid sequence EQPFPQPEQPFPWQP (SEQ ID NO: 2), wherein the N-terminal glutamate is a pyroglutamate and the C-terminal proline is amidated; and
the third peptide comprises the amino acid sequence EPEQPIPEQPQPYPQQ (SEQ ID NO: 3), wherein the N-terminal glutamate is a pyroglutamate and the C-terminal glutamine is amidated.

2. The method of claim 1, wherein the doses in the dose escalation regimen are administered to the subject two times per week, with each dose administered between one to three times before escalation to the next highest dose.

3. The method of claim 1 or 2, wherein the 900 microgram dose in the tolerizing regimen is administered to the subject two times per week, optionally wherein the at least one dose in the tolerizing regimen is self-administered by the patient.

4. The method of claim 1, wherein:
the 1 microgram dose contains one third of a microgram of the first peptide and an equimolar amount of each of the second and third peptides;
the 3 microgram dose contains 1 microgram of the first peptide and an equimolar amount of each of the second and third peptides;
the 9 microgram dose contains 3 micrograms of the first peptide and an equimolar amount of each of the second and third peptides;
the 30 microgram dose contains 10 micrograms of the first peptide and an equimolar amount of each of the second and third peptides;
the 60 microgram dose contains 20 micrograms of the first peptide and an equimolar amount of each of the second and third peptides;
the 90 microgram dose contains 30 micrograms of the first peptide and an equimolar amount of each of the second and third peptides;
the 150 microgram dose contains 50 micrograms of the first peptide and an equimolar amount of each of the second and third peptides;
the 300 microgram dose contains 100 micrograms of the first peptide and an equimolar amount of each of the second and third peptides;
the 450 microgram dose contains 150 micrograms of the first peptide and an equimolar amount of each of the second and third peptides;
the 600 microgram dose contains 200 micrograms of the first peptide and an equimolar amount of each of the second and third peptides;
the 750 microgram dose contains 250 micrograms of the first peptide and an equimolar amount of each of the second and third peptides; and
the 900 microgram dose contains 300 micrograms of the first peptide and an equimolar amount of each of the second and third peptides.

5. (canceled)

6. The method of claim 1, wherein each of the gluten peptide compositions is administered intradermally or subcutaneously, optionally wherein each of the gluten peptide compositions is formulated as a sterile, injectable solution, optionally wherein the sterile, injectable solution is sodium chloride, optionally wherein the sodium chloride is sterile sodium chloride 0.9% USP.

7-9. (canceled)

10. A method for treating Celiac disease in a subject, the method comprising:
administering to the subject at least two different gluten peptide compositions during a dose escalation phase, wherein each gluten peptide composition comprises less than 900 micrograms gluten peptide; and
subsequently administering to the subject during a tolerizing phase a second composition comprising at least 500, 550, 600, 650, 700, 750, 800, 850, or 900 micrograms gluten peptide, wherein:
the first peptide comprises the amino acid sequence ELQPFPQPELPYPQPQ (SEQ ID NO: 1), wherein the N-terminal glutamate is a pyroglutamate and the C-terminal glutamine is amidated; the second peptide comprises the amino acid sequence EQPFPQPEQPFPWQP (SEQ ID NO: 2), wherein the N-terminal glutamate is a pyroglutamate and the C-terminal proline is amidated; and the third peptide comprises the amino acid sequence EPEQPIPEQPQPYPQQ (SEQ ID NO: 3), wherein the N-terminal glutamate is a pyroglutamate and the C-terminal glutamine is amidated, and
optionally, wherein at least one or all of the gluten peptide composition of the dose escalation phase is in an amount different from any of 3, 9, 30, 60, 90, 150, 300, 450, 600 and 750 micrograms of the gluten peptides.

11. The method of claim 10, wherein the at least two different gluten peptide compositions administered during the dose escalation phase are at least 3, 4, 5, 6, 7, 8, 9, 10, 11, 12, 13, 14, or 15 different gluten peptide compositions.

12. The method of claim 10, wherein each of the at least two different gluten peptide compositions is in an amount of 1 to 899 micrograms, with each different gluten peptide composition administered subsequent is in an amount greater than the previous administered different gluten peptide composition, optionally wherein:
(i) the at least two different gluten peptide compositions of the dose escalation phase comprise a first gluten peptide composition in an amount between 1 and 10 micrograms, optionally 1 microgram;
(ii) the at least two different gluten peptide compositions of the dose escalation phase comprise a second gluten peptide composition in an amount between 10 and 75 micrograms;
(iii) the at least two different gluten peptide compositions of the dose escalation phase comprise a third gluten peptide composition in an amount between 50 and 100 micrograms;
(iv) the at least two different gluten peptide compositions of the dose escalation phase comprise a fourth gluten peptide composition in an amount between 75 and 150 micrograms;
(v) the at least two different gluten peptide compositions of the dose escalation phase comprise a fifth gluten peptide composition in an amount between 100 and 300 micrograms;
(vi) the at least two different gluten peptide compositions of the dose escalation phase comprise a sixth gluten peptide composition in an amount between 150 and 500 micrograms;
(vii) the at least two different gluten peptide compositions of the dose escalation phase comprise a seventh gluten peptide composition in an amount between 300 and 750 micrograms; or
(viii) the at least two different gluten peptide compositions of the dose escalation phase comprise an eighth gluten peptide composition in an amount between 500 and 899 micrograms.

13-20. (canceled)

21. The method of claim 12, wherein:
(i) the first, second and/or third gluten peptide composition is administered once or twice; and/or
(ii) the third, fourth, fifth, sixth, seventh and/or eighth gluten peptide composition is administered at least twice.

22. (canceled)

23. The method of claim 10, wherein:
(i) the dose escalation period is at least 3, 3.5, 4, 4.5, 5, 5.5, 6, 6.5, 7, 7.5, 8 or more weeks, and/or
(ii) the tolerizing phase is at least 3, 3.5, 4, 4.5, 5, 5.5, 6, 6.5, 7, 7.5, 8 or more weeks.

24. The method of any one of claims 10-23, wherein the tolerizing phase is at least 3, 3.5, 4, 4.5, 5, 5.5, 6, 6.5, 7, 7.5, 8 or more weeks.

25. The method of claim 10, wherein the subject has a non-homozygous HLA-DQ2.5 genotype.

26. A method for treating Celiac disease in a subject, the method comprising:
administering to the subject at least two different gluten peptide compositions during a dose escalation phase, wherein each gluten peptide composition comprises less than 150 micrograms gluten peptide; and
subsequently administering to the subject during a tolerizing phase a second composition comprising at least 150, 160, 170, 180, 190, 200, 210, 220, 230, 240, 250, 260, 270, 280, 290, or 300 micrograms gluten peptide,
wherein:
the first peptide comprises the amino acid sequence ELQPFPQPELPYPQPQ (SEQ ID NO: 1), wherein the N-terminal glutamate is a pyroglutamate and the C-terminal glutamine is amidated;
the second peptide comprises the amino acid sequence EQPFPQPEQPFPWQP (SEQ ID NO: 2), wherein the N-terminal glutamate is a pyroglutamate and the C-terminal proline is amidated; and
the third peptide comprises the amino acid sequence EPEQPIPEQPQPYPQQ (SEQ ID NO: 3), wherein the N-terminal glutamate is a pyroglutamate and the C-terminal glutamine is amidated, and
optionally, wherein at least one or all of the gluten peptide compositions of the dose escalation phase is in an amount different from any of 3, 9, 30, 60, 90, and 150 micrograms of the gluten peptides.

27. The method of claim 26, wherein the at least two different gluten peptide compositions administered during the dose escalation phase are at least 3, 4, 5, 6, 7, 8, 9 or 10 different gluten peptide compositions.

28. The method of claim 26, wherein each of the at least two different gluten peptide compositions is in an amount of 1 to 149 micrograms, with each different gluten peptide composition administered subsequent is in an amount greater than the previous administered different gluten peptide composition, optionally wherein:
(i) the at least two different gluten peptide compositions of the dose escalation phase comprise a first gluten peptide composition in an amount between 1 and 10 micrograms, optionally 1 microgram;
(ii) the at least two different gluten peptide compositions of the dose escalation phase comprise a second gluten peptide composition in an amount between 10 and 75 micrograms;
(iii) the at least two different gluten peptide compositions of the dose escalation phase comprise a third gluten peptide composition in an amount between 50 and 100 micrograms; or
(iv) the at least two different gluten peptide compositions of the dose escalation phase comprise a fourth gluten peptide composition in an amount between 75 and 149 micrograms.

29-32. (canceled)

33. The method of claim 28, wherein:
(i) the first and/or second gluten peptide composition is administered once or twice, and/or
(ii) the third and/or fourth gluten peptide composition is administered at least twice.

34. (canceled)

35. The method of claim 26, wherein:
(i) the dose escalation period is at least 3, 3.5, 4, 4.5, 5, 5.5, 6, 6.5, 7, 7.5, 8 or more weeks, and/or
(ii) the tolerizing phase is at least 3, 3.5, 4, 4.5, 5, 5.5, 6, 6.5, 7, 7.5, 8 or more weeks.

36. (canceled)

37. The method of claim 1, wherein the subject has a homozygous HLA-DQ2.5 genotype.

38-39. (canceled)

40. The method of claim 10, wherein the gluten peptide compositions of the dose escalation and/or tolerizing phase(s) is/are administered twice a week.

41-47. (canceled)

48. One or more gluten peptide compositions for performing a method as in claim 1.

49. A kit comprising one or more gluten peptide compositions for performing a method as in claim 1.


SEQUENCE LISTING (12 sequences)

SEQ ID NO: 1 - protein, 16 aa, Artificial sequence
  Synthetic polypeptide
ELQPFPQPELPYPQPQ

SEQ ID NO: 2 - protein, 15 aa, Artificial sequence
  Synthetic polypeptide
EQPFPQPEQPFPWQP

SEQ ID NO: 3 - protein, 16 aa, Artificial sequence
  Synthetic polypeptide
EPEQPIPEQPQPYPQQ

SEQ ID NO: 4 - protein, 9 aa, Artificial sequence
  Synthetic polypeptide
PFPQPELPY

SEQ ID NO: 5 - protein, 9 aa, Artificial sequence
  Synthetic polypeptide
PQPELPYPQ

SEQ ID NO: 6 - protein, 9 aa, Artificial sequence
  Synthetic polypeptide
PFPQPEQPF

SEQ ID NO: 7 - protein, 9 aa, Artificial sequence
  Synthetic polypeptide
PQPEQPFPW

SEQ ID NO: 8 - protein, 9 aa, Artificial sequence
  Synthetic polypeptide
PIPEQPQPY

SEQ ID NO: 9 - protein, 9 aa, Artificial sequence
  Synthetic polypeptide
EQPIPEQPQ

SEQ ID NO: 10 - protein, 16 aa, Artificial sequence
  Synthetic polypeptide
  MISC_FEATURE (1)..(1): Pyroglutamate
  MISC_FEATURE (16)..(16): Amidated glutamine
ELQPFPQPELPYPQPQ

SEQ ID NO: 11 - protein, 15 aa, Artificial sequence
  Synthetic polypeptide
  MISC_FEATURE (1)..(1): Pyroglutamate
  MISC_FEATURE (15)..(15): Amidated proline
EQPFPQPEQPFPWQP

SEQ ID NO: 12 - protein, 16 aa, Artificial sequence
  Synthetic polypeptide
  MISC_FEATURE (1)..(1): Pyroglutamate
  MISC_FEATURE (16)..(16): Amidated glutamine
EPEQPIPEQPQPYPQQ